(19) United States
(12) Patent Application Publication
(10) Pub. No.: US 2026/0357331 A1
(43) Pub. Date: Nov. 12, 2026

(54) DEVICES AND METHODS FOR NUCLEIC ACID AMPLIFICATION FROM BIOLOGICAL SAMPLES

(71) Applicant: Testmate Health SA, Vaud (CH)

(72) Inventors: Jenny Shepherd, Cambridge (GB); Sukie Whitehall, Cambridge (GB); Tivadar Mach, Berlin (DE); Wajeeh Chaudhry, Birmingham (GB); Stewart Burr, Cambridge (GB); Lewis Ho, St. Neots (GB); Daniel Haworth, Cambridge (GB); Liam Connelly, Meldreth (GB); Clay Reber, Oakland, CA (US); Kelly Lewis Brezoczky, Los Gatos, CA (US); Thomas Brezoczky, Los Gatos, CA (US); Debkishore Mitra, Fremont, CA (US); Siew-Veena Sahi, Nyon (CH); Nicolas Chartier, Eclagnens (CH); Louis Neveu, Yverdon-les-bains (CH)

(21) Appl. No.: 19/713,717

(22) Filed: Jun. 18, 2026

Related U.S. Application Data
(63) Continuation of application No. PCT/EP2024/088263, filed on Dec. 20, 2024.
(60) Provisional application No. 63/613,051, filed on Dec. 20, 2023.

(51) Int. Cl. C12Q 1/6844; B01L 3/00; B01L 7/00; C12Q 1/6806; C12Q 1/689; C12Q 1/6893

(52) U.S. Cl. CPC C12Q 1/6844; B01L 3/502; B01L 3/52; B01L 7/00; C12Q 1/6806; C12Q 1/689; C12Q 1/6893; B01L 2200/025; B01L 2200/026; B01L 2200/0631; B01L 2200/0647; B01L 2200/0684; B01L 2200/0689; B01L 2200/087; B01L 2200/10; B01L 2200/16; B01L 2300/042; B01L 2300/0672; B01L 2300/0681; B01L 2300/161; B01L 2400/0478; B01L 2400/0487; B01L 2400/06

(57) ABSTRACT

The present disclosure provides systems, devices, and methods for analyte detection and selective dispensing of sample fluid, reagents, aqueous fluid, and/or air within the device. Disclosed herein are sample preparation systems for untrained users or lay persons, comprising a filter and methods for sample filtration, isolation of target analytes, amplification, and detection within the system. Also disclosed herein are methods for urine sample collection.

### CROSS-REFERENCE TO RELATED APPLICATIONS

This application is a continuation of International Application No. PCT/EP2024/088263 filed Dec. 20, 2024, which claims the benefit of U.S. Provisional Patent Application Ser. No. 63/613,051, filed Dec. 20, 2023, the contents of which are hereby incorporated by reference herein.

### BACKGROUND

Conventional detection of analytes, such as pathogenic microorganisms, typically includes collection of a sample by a skilled clinician (or technician), transporting the sample to a suitable laboratory facility or other testing site, and then testing the sample for the target analyte. The inherent challenges with this approach include limited access to clinicians/technicians and/or suitable laboratory facilities or testing sites, risks with specimen chain-of-control and transportation, and delays in obtaining test results for diseases and conditions that have better prognoses when there is prompt diagnosis and treatment. Risks associated with specimen chain-of-control and transportation include: improper handling and preservation of the specimen to be transported, improper transportation to a suitable diagnostic laboratory, risk of contamination, accidental exposure of the specimen or of the people handling the specimen during transportation, and sample degradation (rendering it untestable or compromising the test integrity). Thus, there is a clear need for portable, at-home, analyte-detection devices. These devices should be more streamlined, simple-to-use by laypeople, accurate, reliable, and affordable.

The manufacture of portable, at-home, and field testing devices requires a balance to be struck between complexity of test design, cost of manufacturing, portability, and cost to consumer. Additionally, detection of small volumes can result in lower device sensitivity. These devices often have multiple reagents and other fluids, each serving different purposes in the device reactions (e.g., lysis, purification, isolation, neutralization, rehydration, elution, washing, incubation, mixing, dissolution, precipitation, amplification, and detection), therefore the timing for their release within the devices can be important and can contribute to the expense, overall complexity, and performance of the device.

Examples of devices that are used for controlled reagent dispensing include those disclosed in U.S. Pat. No. 10,207,269, the disclosure of which is herein incorporated by reference in its entirety. U.S. Pat. No. 10,207,269 discloses a reagent dispensing device having a first substrate with one or more chambers, a resistant unit (e.g., blister pack) comprising a reagent, and a pushing unit configured to move along a circular path within the plane parallel or about parallel to the first surface of the first substrate. The pushing unit comprises at least one protrusion, wherein the pushing unit is configured to provide direct or indirect contact with the resistant unit, wherein a first relative movement of the pushing unit in a direction parallel or about parallel to the surface of the first substrate causes at least one protrusion to contact the resistant unit, thereby releasing reagent in the resistant unit into at least one of the one or more first chambers. Some of the drawbacks of this design include the complexity from multiple manufacturing device components, multiple substrates, and a high technical risk at the interfaces between the pushing unit and resistant units. The effectiveness of the device is also highly dependent on the user maintaining the device in correct orientation and consistent direction. Additional embodiments described in that specification include one in which syringes are used to provide pressurization for delivery of reagent fluids, the flow of which is controlled through the use of valves that are separate and distinct from those that utilize syringes. The present disclosure provides a more streamlined, less complex sample preparation device that allows for controlled, selective dispensing of reagents. In some embodiments, the present disclosure provides a sample preparation device that utilizes one or more plungers or a plurality of plungers to create a plurality of chambers (some of which can comprise reagent) and the plungers serve a dual purpose of operating like a valve ((i) moving to occlude or open a conduit to another chamber for selective dispensing, and (ii) moving to pressurize and dispense contents of the plurality of chambers that they form). This simplifies the device structure while maintaining the function of controlled, selective dispensing.

Many preexisting reagent dispensing devices, including those disclosed in U.S. Pat. No. 10,207,269, are significantly limited in the sample and/or reagent volumes they can process and are not optimal for accommodating dissimilar reagent volumes. In contrast, the presently disclosed device has the technical advantage of being capable of processing larger sample and/or reagent volumes, e.g., in some embodiments, about 2-5 mL. They also have the technical advantage of easily accommodating dissimilar reagent volumes (e.g., in some embodiments, the system comprises about 0.5 mL lysis buffer and about 5 mL water).

### SUMMARY

The subject disclosure provides devices and methods that are useful for rapid detection of pathogens, cancerous cells, and precancerous cells in a biological sample. In some embodiments, the pathogens are those capable of causing a sexually transmitted disease or urinary tract infection. The devices and methods are also useful for detection of cancer or precancerous conditions.

The present disclosure provides a device offering significant improvement in sample analyte detection and ease of use. In some embodiments, the significantly improved devices have two or more chambers, wherein at least one of the two or more chambers comprises multiple plungers that form a plurality of chambers therein. In some embodiments, the plurality of chambers can each comprise a reagent, aqueous fluid, or air. The multiple plungers have the dual function of (i) moving to occlude or open a conduit to another chamber, such that the contents of the plurality of chambers can be selectively dispensed (e.g., a valve-like function), and (ii) moving to pressurize and dispense contents of the plurality of chambers. The dual-functional use of plungers is one of many features that differentiates the presently disclosed devices from pre-existing reagent dispensing devices in the field (e.g., those disclosed in U.S. Pat. No. 10,207,269, the disclosure of which is herein incorporated by reference in its entirety).

Additional technical advantages of the presently disclosed devices is their portability, allowing for at-home testing, and/or sampling and testing without the need for a clinician or laboratory specialist. The devices are designed, for example, for simplified and more cost-effective manufacturing, requiring fewer parts during manufacturing assembly than reagent dispensing devices, e.g., those disclosed in U.S. Pat. No. 10,207,269, the disclosure of which is herein incorporated by reference in its entirety.

In some embodiments, a sample preparation device for processing biological samples for testing for a disease or condition is presented. The sample preparation device may comprise:
(a) a sample preparation body comprising:
   (i) a sample chamber for holding sample fluid,
   (ii) a filter housing fluidically connected to the sample chamber, optionally wherein the filter housing comprises a filter, and
   (iii) a second chamber comprising one or more plungers disposed therein to form a plurality of chambers, wherein the plurality of chambers each comprises at least one of a reagent, aqueous fluid, or air, wherein the second chamber comprises an opening fluidically connecting the filter housing and the second chamber, wherein the opening is configured to be opened and closed upon movement of the one or more plungers through the second chamber so as to selectively dispense contents of the plurality of chambers into the filter housing and through the filter housing; and
(b) a cap comprising a filter plunger, wherein the cap is configured for mounting on an open end of the sample preparation body, and wherein the filter plunger is configured to compress the sample fluid through the filter housing, optionally through though the filter in the filter housing.

In some embodiments, the second chamber comprises two or more plungers.

In some embodiments, the sample preparation device has a reception configuration and a preparation configuration, the device being movable from the reception configuration, in which a sample fluid can be introduced into the sample preparation body, to the preparation configuration, in which the sample preparation body is closed by assembly of the cap to the sample preparation body and the user compresses the cap against the sample preparation body to actuate the one or more plungers.

In some embodiments, the sample chamber is disposed within the second chamber, optionally, wherein the sample chamber and the second chamber are concentric. In some embodiments, the sample preparation device comprises a waste chamber.

In some embodiments, the sample preparation device comprises a prepared sample chamber comprising (i) a breakable barrier and (ii) an outlet port. In some embodiments, the prepared sample chamber is disposed within the waste chamber, and the breakable barrier seals the prepared sample chamber from the waste chamber. In some embodiments, the sample chamber comprises an outlet fluidically connecting the sample chamber and the filter housing, optionally wherein the outlet comprises a breakable barrier. In some embodiments, the filter housing comprises an outlet (optionally a tapered outlet). In some embodiments the outlet is a hollow piercing member. In some embodiments, the outlet is aligned to operatively connect with the breakable barrier in a configuration, thereby breaking the prepared sample chamber's breakable barrier and fluidically connecting the filter housing to the prepared sample chamber. In some embodiments, the outlet is configured to operatively connect with the breakable barrier, optionally upon further closure of the cap, thereby breaking the prepared sample chamber's breakable barrier and fluidically connecting the filter housing to the prepared sample chamber.

In some embodiments, the sample preparation device comprises a valve configured such that when a user actuates the valve, the valve operatively couples the filter housing to the prepared sample chamber, thereby fluidically connecting the filter housing to the prepared sample chamber. In some embodiments the prepared sample chamber is configured to be compressed against the filter housing, thereby breaking the prepared sample chamber's breakable barrier and fluidically connecting the filter housing to the prepared sample chamber.

In some embodiments at least one of the plurality of chambers comprises a reagent. In some embodiments the reagent comprises, e.g., one or more of a washing buffer, a lysis buffer, a neutralization buffer, an elution buffer, or a rehydration buffer.

In some embodiments the reagent comprises a lysis buffer. In some embodiments, at least one of the plurality of chambers comprises aqueous fluid. In some embodiments at least one of the plurality of chambers comprises air.

In some embodiments, a sample preparation system for processing biological samples for testing for a disease or condition is presented. In some embodiments, a sample preparation system comprises
(a) a sample preparation body comprising an inner body, an outer body, a filter housing, a waste chamber, and a reagent chamber,
   wherein the inner body comprises a sample chamber for holding sample fluid,
   wherein the filter housing is fluidically connected to the sample chamber,
   wherein the waste chamber comprises a prepared sample chamber,
   wherein the prepared sample chamber comprises (i) a breakable barrier sealing it from the waste chamber and (ii) an outlet port,
   wherein the filter housing comprises (i) an first end, (ii) a filter, and (iii) a second end,
   wherein the second end comprises an outlet (optionally a tapered outlet) configured to allow filtrate flow therethrough,
   wherein the outer body comprises one or more plungers disposed therein to form at least one aqueous fluid chamber and/or at least one air chamber;
(b) an amplification module (also referred to as “amp module”) operatively coupled to the sample preparation body and adjacent to the prepared sample chamber,
   wherein the amplification module comprises an interface and a reagent plunger
   configured to move within the reagent chamber,
      wherein the outlet port of the prepared sample chamber is configured to receive the interface, thereby fluidically connecting the prepared sample chamber and the amplification module, and
      wherein the reagent plunger is configured to move within the reagent chamber; and
(c) a cap configured for mounting on the open end of the sample preparation body,
   wherein the cap comprises a filter plunger aligned to enter the open end of the sample chamber when the sample preparation body is closed by the assembly of the cap to the sample preparation body;
wherein the sample preparation system has a reception configuration, a filtering configuration, and a reagent configuration, the system being movable from the reception configuration in which the sample fluid can be introduced into the sample chamber, to the filtering configuration in which sample preparation body is closed by the assembly of the cap to the sample preparation body, to the reagent configuration,
   wherein in the filtering configuration, the filter plunger pressurizes the sample chamber, thereby driving the sample fluid from the sample chamber through the filter housing and into the waste chamber, and wherein further compressing the cap against the sample preparation body moves the one or more plungers to open and close the outer body (optionally through an opening on the outer body wall) so as to selectively dispense the content of the at least one aqueous fluid chamber and/or the at least one air chamber through the filter housing and into the waste chamber,
   wherein in the reagent configuration, further compressing the cap against the sample preparation body operatively couples the second end and prepared sample chamber, thereby breaking the breakable barrier and fluidically connecting the reagent chamber, the filter housing, and the prepared sample chamber, and further compressing the cap moves the reagent plunger into the reagent chamber, thereby driving the contents of the reagent chamber through the filter housing to the prepared sample chamber.

In some embodiments, the reagent chamber comprises a second plunger, thereby forming a plurality of chambers each having one or more reagents, aqueous fluid, or air. In some embodiments, in the reagent configuration, the cap is compressed so as to selectively dispenses the contents of the plurality of chambers through the filter housing and prepared sample chamber. In some embodiments, the plurality of chambers comprise at least one chamber having a reagent and at least one chamber having air. In some embodiments, as the reagent plunger moves into the reagent chamber, the contents of a chamber having air are driven through the filter housing after the contents of a chamber having a reagent are driven through the filter housing. In some embodiments, the contents of the air chamber are driven through the filter housing and the prepared sample chamber after the contents of the aqueous fluid chamber are driven through the filter housing.

In some embodiments, the filter of the filter housing has a pore size suitable for allowing fluid flow therethrough and for obstructing the flow of cells. In some embodiments, the cells are bacteria, fungi, or protists. In some embodiments, the cells are cancerous cells and/or precancerous cells.

In some embodiments, the reagent chamber comprises a vent. In some embodiments, the vent has passively tunable porosity. In some embodiments, the cap is a screw cap. In some embodiments, the reagent chamber does not have a vent, e.g., is sealed on one side by a plunger (e.g. one of the plungers therein). In some embodiments that sealing plunger cannot be fully ejected from reagent chamber (e.g. because of a mechanical feature, e.g. stop, controlling maximal movement in the direction fluid is move to dispense to subsequent chambers). In some embodiments, the cap comprises a first attachment element and the sample preparation body has a second attachment element. In some embodiments, the first attachment element comprises threading and the second attachment element comprises reciprocating threading configured to slidably receive the threading. In some embodiments, the reciprocating threading is configured to slidably receive ½ to 10 complete rotations of the threading of the cap. In some embodiments, the reciprocating threading is configured to slidably receive at least 3 complete rotations of the threading of the cap to complete the filtering configuration or the preparation configuration. In some embodiments, the reciprocating threading is configured to slidably receive at least 5 complete rotations of the threading of the cap to complete the filtering configuration or the preparation configuration. In some embodiments, the reciprocating threading is configured to slidably receive 3 to 5 complete rotations of the threading of the cap to complete the filtering configuration or the preparation configuration.

In some embodiments, a sample preparation device for processing biological samples for testing for a disease or condition is presented, comprising:
a sample preparation body comprising:
   (i) a sample chamber for holding sample fluid,
   (ii) a filter housing fluidically connected to the sample chamber;
   (iii) a second chamber comprising one plunger disposed therein to form a plurality of chambers, wherein the plurality of chambers each comprise at least one of a reagent, aqueous fluid, or air, and wherein the second chamber comprises an opening fluidically connecting the sample chamber and the second chamber, wherein the opening is configured to be opened and closed upon movement of the plunger through the second chamber so as to selectively dispense contents of the plurality of chambers into the sample chamber and through the filter;
   (iv) a cap comprising a filter plunger, wherein the cap is configured for mounting on an open end of the sample preparation body, wherein the filter plunger is configured to compress the sample fluid against the filter; and
   (v) a reagent chamber comprising one plunger disposed therein to form a plurality of chambers, wherein the plurality of chambers each comprise (a) one chamber of a reagent, aqueous fluid, or air and (b) one waste chamber.

In some embodiments, the second chamber comprises two or more chambers disposed therein to form a plurality of chambers. In some embodiments, the reagent chamber comprises two or more plungers disposed therein to form a plurality of chambers. In some embodiments, the plurality of chambers in the reagent chamber comprise (a) one chamber of reagent and (b) one waste chamber. In some embodiments, the outlet comprises a hydrophilic material. In some embodiments, the breakable barrier comprises a hydrophilic material. In some embodiments, the outlet comprises a material having a hardness that is at least 2 times a hardness of a material comprising the breakable barrier, such that the outlet provides for a clear puncture of the breakable seal with repeatable geometry and without deformation of the puncturing element. In some embodiments, the cap comprises knurlings and/or one or more finger-grip indentations. In some embodiments, the outer surface of the sample preparation body and/or the amplification module comprises one or more alignment features.

In some embodiments, the cap, the sample preparation tube and/or the amplification module comprise irreversible coupling features (such as locking detents, cantilever snap features, lock-out release arm, ratchet features, sawtooth ratchets, ratchet and pawl features). In some embodiments, the sample fluid comprises a urine sample, a saliva sample, and/or a mouthwash sample. In some embodiments, the sample fluid comprises a urine sample. In some embodiments, the sample fluid comprises a fecal sample or mucosal sample. In some embodiments, the sample fluid comprises an analyte. In some embodiments, the disease or condition is a sexually transmitted disease, an oncological condition, a fungal infection, and/or a bacterial infection. In some embodiments, the filter comprises an affinity capture reagent membrane. In some embodiments, the filter comprises an anion exchange membrane. In some embodiments, the filter comprises a cation exchange membrane. In some embodiments, the filter comprises a target-specific binding moiety.

In some embodiments, the filter comprises protein A. In some embodiments, the filter comprises protein G. In some embodiments, the filter comprises protein L. In some embodiments, the filter is infused with a salt. In some embodiments, the filter has a pore size no greater than 0.7 μm, 0.6 μm, 0.5 μm, 0.45 μm, 0.4 μm, 0.2 μm, or 0.22 μm. In some embodiments, where a filter is described, filter housing is also contemplated.

In some embodiments the reagent comprises a lysis buffer. In some embodiments, the lysis buffer comprises at least one of sodium hydroxide, sodium dihydrogen phosphate, disodium hydrogen phosphate, Tris, HEPES, and detergent. In some embodiments, the reagent comprises a neutralization reagent. In some embodiments, the neutralization reagent comprises an alkaline reagent and/or wherein the neutralization buffer comprises one or more of Tris, Tris-HCl, and ethylenediaminetetraacetic acid (EDTA). In some embodiments, the amplification module comprises LAMP primers.

In some embodiments, a method of determining whether a user has a disease or condition is presented, comprising:
(i) providing a sample preparation system or device of any one of the preceding claims;
(ii) providing a biological sample;
(iii) filtering at least a portion of the biological sample through the filter, thereby retaining an analyte, comprising nucleic acid molecules, on the filter;
(iv) applying at least one reagent to the filter, thereby producing a lysate, wherein the one reagent comprises a lysis buffer;
(v) amplifying the nucleic acid molecules of the analyte, and based on said amplifying;
(vi) determining the presence or absence of the analyte.

In some embodiments, a method of determining whether a user has a disease or condition is presented, the method comprising:
(i) providing a sample preparation device having a filter housing that comprises a filter and is fluidically connected to the sample chamber;
(ii) providing a urine sample that was collected by having the user micturate into a sample collector by initiating and then stopping their flow of urine into the sample collector, wherein the sample collector has a volume of no more than about 70 mL, optionally about 20 mL, 30 mL, 40 mL, 50 mL or 60 mL;
(iii) pouring a predetermined volume from the urine sample into the sample preparation device and filtering the predetermined volume, thereby retaining an analyte on the filter, wherein the predetermined volume is no more than about 5 mL;
(iv) applying a lysing agent to the filter, thereby producing a lysate comprising nucleic acid molecules;
(v) amplifying nucleic acid molecules, and based on said amplifying;
(vi) determining the presence or absence of a pathogen, cancerous cell, and/or precancerous cell.

In some embodiments, a method of determining whether a user has a disease or condition is presented, the method comprising:
(i) providing sample preparation device or system of any one of claims 1-61;
(ii) providing a urine sample that was collected by having the user micturate into a sample collector by initiating and then stopping their flow of urine into the sample collector, wherein the sample collector has a volume of no more than about 70 mL, optionally about 20 mL, 30 mL, 40 mL, 50 mL or 60 mL;
(iii) pouring a predetermined volume from the urine sample into the sample chamber and filtering the predetermined volume, thereby retaining an analyte on the filter, wherein the predetermined volume is no more than about 5 mL;
(iv) applying a lysing agent to the filter, thereby producing a lysate comprising nucleic acid molecules;
(v) amplifying nucleic acid molecules, and based on said amplifying;
(vi) determining the presence or absence of a pathogen, cancerous cell, and/or precancerous cell.

In some embodiments, the sample collector has a volume of no more than about 15 mL. In some embodiments, the sample collector has a volume of no more than about 10 mL. In some embodiments, the predetermined volume is about 3 mL. In some embodiments, the predetermined volume is about 4 mL. In some embodiments, the predetermined volume is about 5 mL.

In some embodiments, the inner surface of the sample collector comprises at least two indicators. In some embodiments, the pathogen is a bacterium, fungus, protist, or virus that is capable of causing sexually transmitted infections. In some embodiments, the pathogen is a bacterium, fungus, protist, or virus that is capable of causing a urinary tract infection.

In some embodiments, determining presence or absence of a pathogen, cancerous cell, and/or precancerous cell comprises using a lateral flow assay (LFA).

### BRIEF DESCRIPTION OF THE DRAWINGS

These and other features, aspects, and advantages of the present disclosure will become better understood with regard to the following description, and accompanying drawings, where:

FIG. 1 is a diagram showing an exemplary sample preparation system 100, including an amplification module 104 for analyte amplification and detection, in accordance with an embodiment.

FIG. 2 is a diagram showing an exemplary sample preparation system 100, in accordance with an embodiment.

FIG. 3 is a diagram showing an exemplary sample preparation to analyte detection workflow, in accordance with an embodiment.

FIG. 4 is a diagram showing an exemplary sample preparation device 102, in accordance with an embodiment.

FIG. 5 is a diagram showing an exemplary workflow for use of the device in FIG. 4.

FIG. 6 is a diagram showing an exemplary sample preparation device 102, wherein the device comprises stacked cups, in accordance with an embodiment.

FIG. 7 is a diagram showing an exemplary workflow for use of the device in FIG. 6.

FIG. 8 is a diagram showing an exemplary sample preparation device 102, wherein the device comprises bags, in accordance with an embodiment.

FIG. 9 is a diagram showing an exemplary workflow for use of the device in FIG. 8.

FIG. 10 is a diagram showing an exemplary sample preparation device 102, wherein the device comprises a multi-part plunger and filter plenum, in accordance with an embodiment.

FIG. 11 is a diagram showing an exemplary workflow for use of the device in FIG. 10.

FIG. 12 is a diagram showing an exemplary sample preparation device 102, wherein the device comprises a cap comprising a plurality of chambers, in accordance with an embodiment.

FIGS. 13A and 13B are diagrams showing an exemplary sample preparation device 102 and a component thereof, wherein the device comprises bags 800 (e.g. FIG. 13B), in accordance with an embodiment.

FIG. 14 is a diagram showing an exemplary sample preparation system, in accordance with an embodiment. Shown in the figure are air 1400, aqueous fluid 1402, lysis buffer 1404, and resuspension buffer 1406 in their respective chambers.

FIG. 15 is a diagram showing an exemplary overflow prevention system, wherein the sample chamber widens proximal to a user and a plunger (grey) seals out the overflow, in accordance with an embodiment.

FIG. 16 is a plot showing pressure measurements for an MCE filter.

FIG. 17 are plots showing pressure measurements for MCE filters and GF/F filters.

FIG. 18 is a diagram showing series of instructions for a urine collection workflow, in accordance with an embodiment.

FIGS. 19A and 19B is a diagram showing the rounds (FIG. 19A) for the study and associated devices. FIG. 19B shows exemplary device improvements utilized for round 2.

FIGS. 20A-20D are plots showing user feedback during urine collection to test experiments. FIG. 20A: when asked how their overall experience of peeing just a small amount of urine in a collection cup. FIG. 20B: when asked how acceptable it was to be asked to pee a very small amount in the collection cup. FIG. 20C: when asked how clear and understandable step 1 instructions were. FIG. 20D: when asked their overall experience of conducting the test.

FIG. 21 is a diagram showing an exemplary workflow for use of a sample preparation device 102 according to an embodiment.

FIGS. 22A-22C are diagrams showing exemplary sample preparation systems 100.

The figures depict various embodiments of the present disclosure for purposes of illustration only. One skilled in the art will readily recognize from the following discussion that alternative embodiments of the structures and methods illustrated herein can be employed without departing from the principles of the disclosure described herein.

### DETAILED DESCRIPTION

### I. Definitions

Before the present disclosure is described in greater detail, it is to be understood that the present disclosure is not limited to particular embodiments described, and as such can, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges and are also encompassed within the disclosure, subject to any specifically excluded limit in the stated range. Where the stated range includes one or both of the limits, ranges excluding either or both of those included limits are also included in the disclosure.

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or assembly that comprises a list of elements is not necessarily limited to only those elements but can include other elements not expressly listed or inherent to such process, method, article, or assembly. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “about” can mean within an acceptable error range for the particular value as determined by one of ordinary skill in the art, which will depend in part on how the value is measured or determined, e.g., the limitations of the measurement system. For example, “about” can mean plus or minus 5%, 10%, or 20%, per the practice in the art. In certain embodiments, the term “about” refers to being within manufacturing tolerance levels as known by one of ordinary skill in the art (e.g., AS 1163, EN 10219, ASTM A500 or G3444/G3466 tolerance level standards). In case of doubt, encompassed within the term “about” are numbers that are insignificantly different from the stated number.

Where ranges and/or subranges of values are provided, the ranges and/or subranges can include the endpoints of the ranges and/or subranges.

In addition, it is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “proximal end” refers to the end of a device or instrument that is nearer to the user or operator. In contrast, “distal end” refers to the end of a device or instrument that is farther away from the user or operator.

Additionally, certain embodiments of the disclosed devices and/or associated methods can be represented by drawings which can be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

Some embodiments can be described using the expression “coupled” and “connected” along with their derivatives. For example, some embodiments can be described using the term “coupled” to indicate that two or more elements are in direct physical or electrical contact. In some embodiments, the term “coupled” is also used to describe two or more elements that are in active fluidic connection. The term “coupled,” however, can also mean that two or more elements are not in direct contact with each other, but yet still co-operate or interact with each other. The embodiments are not limited in this context unless otherwise explicitly stated.

“Operatively coupled,” “operatively connected,” and “operatively attached” as used herein means connected in a specific way that allows the disclosed devices to operate and/or methods to be carried out effectively in the manner described herein. For example, operatively coupling can include removably coupling or fixedly coupling two or more aspects (e.g., the sample preparation body and the amplification module 104). Operatively coupling can also include fluidically coupling two or more aspects. For example, in some embodiments, a sample preparation body and the amplification module 104 are in a reagent configuration, as disclosed herein. Operatively coupling can also include mateably coupling two or more components (e.g., the sample preparation body and the cap). In some embodiments, operative coupling refers to the different forms of engagement, such as (but not limited to), screwing-on, twisting-together, snapping-on, press-fitting, screwing together, sliding together, etc., of two of more aspects (e.g., the sample preparation body and the cap). In some embodiments, the engagement refers to the sample preparation body being closed by assembly of the cap to the sample preparation body. All these forms of engagement result in a compression of the cap against the sample preparation body and, in some embodiments, result in pressurizing of the contents of the sample preparation body (e.g., of the sample chamber).

As used herein, the term “sample preparation body” is not limited by shape. In some embodiments, it can be a cylindrical tube.

As used herein, the terms “affixed” and “attached” are used interchangeably to describe a component being attached, joined, stuck, or fastened to another component (e.g., to a cap). For example, in some embodiments, the sample preparation system or device comprises a cap having a plunger affixed to it and said plunger is referred to as the “filter plunger.”

A “reagent” can be any chemical, including organic compounds and inorganic compounds and combinations thereof. It can be provided in gaseous, solid, or liquid form, or any combination thereof, it can be in lyophilized form, and/or it can be a component of a solution or a suspension. In some embodiments, a reagent comprises a buffer useful in methods of detecting analytes in a sample fluid or retentate. In some embodiments, a reagent comprises an anticoagulant, a diluent, a buffer, a specific binding moiety, detectable labels, enzymes and the like. In some embodiments, a reagent can also include an extractant, such as a buffer or a chemical, to extract an analyte from a sample or a sample collection device. In some embodiments, a reagent comprises a washing buffer, a lysis buffer, a neutralization buffer, or a rehydration buffer.

As used herein, the term “passively tunable porosity” refers to the ability of having a first conformation in which one or more gasses (such as air) can pass through the pores, and a second conformation in which fluids (including the one or more gasses and liquids) are minimized or prevented from passing through the pores, and whereby, an element having passively tunable porosity proceeds automatically from the first to the second conformation upon contact with a liquid. The presence of a vent allows for the movement of fluids through a chamber comprising said vent. The presence of a vent having passively tunable porosity, can minimize or prevent the unintended loss of liquids, e.g., by expulsion or evaporation, from the chamber comprising said vent. In some embodiments, a chamber of the devices disclosed herein, e.g., a chamber comprising a plunger, comprise vents.

The term “passively tunable porosity” also refers to proceeding from the first conformation to the second conformation passively, e.g., automatically without user interaction, upon contacting one or more liquids, such as liquids including a biological sample, with the vent or a portion thereof.

For example, in some embodiments, the vent comprises a hydrogel having a passively tunable porosity. Such a hydrogel can be capable of swelling and reducing the porosity of the porous polymer matrix upon contact with a liquid, e.g., an aqueous liquid.

Materials with passively tunable porosity include, without limitation, one or more polymer matrix, such as a porous polymer matrix, such as polyethylene, a hydrogel, such as carboxymethyl cellulose. Other materials can also be composed to include saccharides, proteins, deliquescent materials, nylon, ABS, polycarbonate, and Poly(methyl methacrylate), other hygroscopic materials, or any combinations thereof.

As used herein, an “alignment feature” refers to a feature (e.g., protruding feature or surface marking (e.g., lines, arrows, circles, etc.)) on the sample preparation body, the cap, and/or the amplification module that facilitates proper alignment when coupling system components. Non-limiting examples of these alignment features include rib, tabs, hooks, notches, and nubs.

As used herein, the term “biological sample” is a sample containing a quantity of organic material, e.g., one or more organic molecules, such as one or more nucleic acids e.g., DNA and/or RNA or portions thereof, which can be taken from a subject. In some embodiments, the term “biological sample” and “sample fluid” are used interchangeably. In some embodiments, the sample fluid comprises a urine sample, a saliva sample, and/or a mouthwash sample. In some embodiments, the sample fluid is formed from resuspension of a solid or semi-solid sample (e.g., fecal sample or mucosal sample) in solution. In some embodiments, the sample fluid comprises a fecal sample or mucosal sample. In some embodiments, the sample fluid is formed from resuspension any sample from a user likely to contain an analyte of interest (e.g., free nucleic acids, nucleic acids for a pathogen, a cancerous cell, or precancerous cell), as would be appreciated by one of ordinary skill in the art. In some embodiments, the cancerous and precancerous cells are identified based on expression profiles, presence or absence of certain biomarkers (e.g., cancer markers or tumor markers). In some embodiments, the sample fluid is selected from the group consisting of pus, blood sample, skin sample, etc. e.g. In some embodiments, the sample fluid is selected from the group consisting of blood, urine, semen, vaginal discharge, a vaginal swab, a nasal swab, a nasopharyngeal swab, a mid-turbinate swab, fecal sample, tears, fluid excreted at wound sites or sites of inflammation, or any other clinical material that contains nucleic acids or proteins.

The biological sample can be obtained using any appropriate vehicle for use to transfer the sample in solution into the sample collector. For example, a biological sample can be collected with a swab and then suspended in solution for use in the disclosed devices. As one of skill in the art will appreciate, the biological sample can be obtained by means other than a swab, e.g., by a syringe or a collection vessel.

As used herein, the term “nucleic acid” refers to any molecule, preferably a polymeric molecule, incorporating units of ribonucleic acid, deoxyribonucleic acid or an analog thereof. The nucleic acid can be either single-stranded or double-stranded. In some embodiments, nucleic acids refers to cell free nucleic acids (e.g., circulating cell-free DNA).

In some embodiments, the term “disease or condition” refers to a sexually transmitted disease. In some embodiments, the term “disease or condition” refers to a urinary tract infection. In some embodiments, the term “disease or condition” refers to a cancer or precancerous conditions. In some embodiments, the term “disease or condition” refers to any condition caused by a pathogen (e.g., a bacterium, fungus, protist, or virus/viral agent).

A biological sample can be collected from a subject. In certain embodiments, a subject is a “mammal” or a “mammalian” subject, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the subject is a human. The term “humans” can include human subjects of both genders and at any stage of development (e.g., fetal, neonates, infant, juvenile, adolescent, and adult), where in certain embodiments the human subject is a juvenile, adolescent or adult. While the devices and methods described herein can be applied in association with a human subject, it is to be understood that the subject devices and methods can also be applied in association with other subjects, that is, on “non-human subjects.”

As used herein, the term “user” refers to a human subject.

As used herein, the term “filter housing” refers to a structure capable of retaining a filtering material and capable of filtering a fluid. In some embodiments, a filter housing can be formed or otherwise structured to be positionable within a sample preparation device 102 such that sample fluid or other fluids contemplated herein can travel from their respective chambers (or receptacles, e.g., a sample chamber) through the filter housing-thereby being filtered. In some embodiments, the filter housing comprises a filter. In some embodiments, a portion of the filter housing is a filter. In some embodiments, a filter comprises a porous material or a series of layers. In some embodiments a filter is sintered. In some embodiments, a filter comprises fritted material.

As used herein, the term “aqueous fluid” refers to a liquid that substantially comprises water. Aqueous fluid may comprise about 30% water, about 40% water, about 50% water, about 60% water, about 70% water, about 80% water, about 90% water, or greater than about 90% water. In some embodiments, an aqueous fluid is water. In some embodiments, an aqueous filter is a buffer. In some embodiments, an aqueous fluid comprises saline.

Any terms not directly defined herein shall be understood to have the meanings commonly associated with them as understood within the art of the disclosure. Certain terms are discussed herein to provide additional guidance to the practitioner in describing the compositions, devices, methods and the like of aspects of the disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

### II. Sample Preparation System

In some embodiments, the present disclosure provides a sample preparation device comprising: a sample preparation body 400 comprising (i) a sample chamber 410 for holding sample fluid (ii) a filter housing 416 fluidically connected to the sample chamber; and (ii) a second chamber 402 comprising (a) one plunger 414, (b) one or more plungers 414, or (c) two or more plungers 414, disposed therein to form a plurality of chambers 412. In some embodiments, the plurality of chambers each comprise at least one of a reagent, aqueous fluid, or air.

In some embodiments, the present disclosure provides a sample preparation device comprising: a sample preparation body comprising (i) a sample chamber for holding sample fluid, wherein the sample chamber comprises a filter; and (ii) a second chamber comprising (a) one plunger, (b) one or more plungers, or (c) two or more plungers, disposed therein to form a plurality of chambers, wherein the plurality of chambers each comprise at least one of a reagent, aqueous fluid, or air.

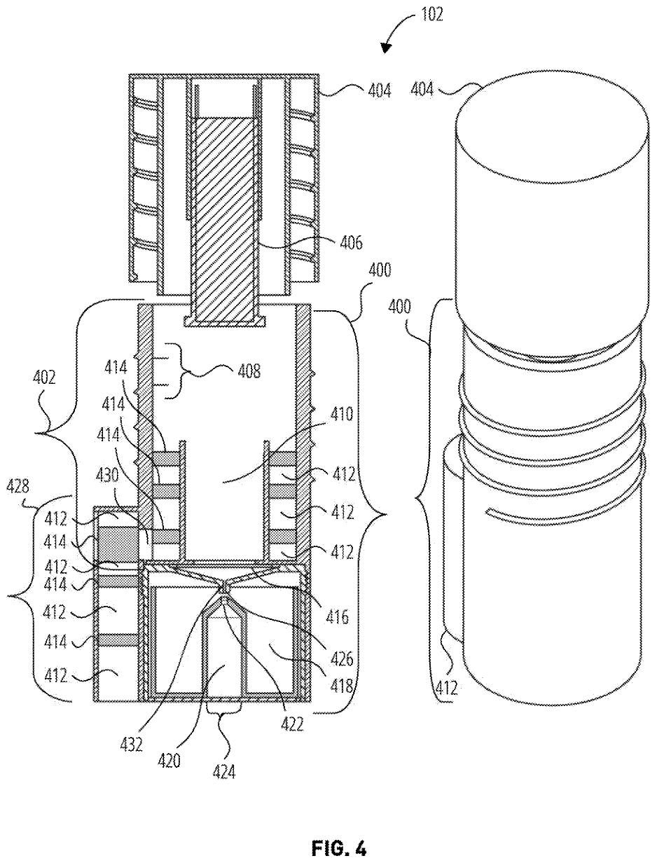

In some embodiments, the second chamber comprises an opening 430 and the opening is configured to be opened and closed (e.g., exposed and covered, exposed and occluded, etc.) upon movement of the plunger, the one or more plungers, or two or more plungers through the second chamber so as to selectively dispense contents of the plurality of chambers into the filter housing, optionally through the filter. In some embodiments, it is so as to dispense into the sample chamber, when the sample chamber comprises a filter. In FIG. 4, a filter housing is shown with an outlet 426 and a channel 432 therethrough.

In some embodiments, the sample preparation body further comprises a reagent chamber 428 as described herein. In some embodiments, the reagent chamber comprises one or more plungers disposed therein to form a plurality of chambers, wherein the plurality of chambers each comprise at least one of a reagent, aqueous fluid, or air. In some embodiments, the plurality of chambers within the reagent chamber each comprise at least one of a reagent, aqueous fluid, or air.

In some embodiments, the present disclosure provides a sample preparation device for processing biological samples for testing for a disease or condition, the device comprising: a sample preparation body comprising (a) a sample preparation body comprising: (i) a sample chamber for holding sample fluid, (ii) a filter housing that comprises a filter and is fluidically connected to the sample chamber, and (iii) a second chamber comprising (I) one, (II) one or more plungers, or (III) two or more (a plurality) of plungers disposed therein to form a plurality of chambers. In some embodiments, the plurality of chambers each comprises at least one of a reagent, aqueous fluid, or air, wherein the second chamber comprises an opening fluidically connecting the filter housing and the second chamber. In some embodiments, the opening is configured to be opened and closed upon movement of the one or more plungers through the second chamber so as to selectively dispense contents of the plurality of chambers, e.g., into the filter housing, e.g., through the filter housing, e.g. through the filter. In some embodiments, the sample preparation body also comprises a cap 404 comprising a filter plunger 406, wherein the cap is configured for mounting on an open end of the sample preparation body, and wherein the filter plunger is configured to compress the sample fluid against the filter through the filter housing.

In some embodiments, the present disclosure provides a sample preparation device for processing biological samples for testing for a disease or condition, the device comprising: a sample preparation body comprising (i) a sample chamber for holding sample fluid, wherein the sample chamber comprises a filter; (ii) a second chamber comprising two or more plungers disposed therein to form a plurality of chambers, wherein the plurality of chambers each comprise at least one of a reagent, aqueous fluid, or air, and wherein the second chamber comprises an opening fluidically connecting the sample chamber and the second chamber, wherein the opening is configured to be opened and closed upon movement of the two or more plungers through the second chamber so as to selectively dispense contents of the plurality of chambers into the sample chamber and through the filter; and (iii) a cap comprising a filter plunger, wherein the cap is configured for mounting on an open end of the sample preparation body, wherein the filter plunger is configured to compress the sample fluid against the filter.

In some embodiments, the present disclosure provides a sample preparation device for processing biological samples for testing for a disease or condition, the device comprising: a sample preparation body comprising (i) a sample chamber for holding sample fluid, wherein the sample chamber comprises a filter; (ii) a second chamber comprising one plunger disposed therein to form a plurality of chambers, wherein the plurality of chambers each comprise at least one of a reagent, aqueous fluid, or air, and wherein the second chamber comprises an opening fluidically connecting the sample chamber and the second chamber, wherein the opening is configured to be opened and closed upon movement of the plunger through the second chamber so as to selectively dispense contents of the plurality of chambers into the sample chamber and through the filter; and (iii) a cap comprising a filter plunger, wherein the cap is configured for mounting on an open end of the sample preparation body, wherein the filter plunger is configured to compress the sample fluid against the filter.

In some embodiments, the present disclosure provides a sample preparation device for processing biological samples for testing for a disease or condition, the device comprising: a sample preparation body comprising (i) a sample chamber for holding sample fluid, wherein the sample chamber comprises a filter; (ii) a second chamber comprising one or more plungers disposed therein to form a plurality of chambers, wherein the plurality of chambers each comprise at least one of a reagent, aqueous fluid, or air, and wherein the second chamber comprises an opening fluidically connecting the sample chamber and the second chamber, wherein the opening is configured to be opened and closed upon movement of the one or more plungers through the second chamber so as to selectively dispense contents of the plurality of chambers into the sample chamber and through the filter; and (iii) a cap comprising a filter plunger, wherein the cap is configured for mounting on an open end of the sample preparation body, wherein the filter plunger is configured to compress the sample fluid against the filter.

In the present disclosure, it is contemplated that a sample preparation body described as comprising a sample chamber comprising a filter can instead have a sample chamber and a filter housing, wherein the filter housing is fluidically connected to the sample chamber. In some embodiments, where a filter housing is present, the sample chamber has an opening configured for fluidic connection between the sample chamber and filter housing in one or more configurations. In some embodiments, an opening on the sample chamber comprises a breakable barrier or a valve. In some embodiments, the breakable barrier can be broken to fluidically connect the sample chamber and filter housing. In some embodiments, the valve can be actuated, e.g., by a user, to fluidically connect the sample chamber and the filter housing in at least one configuration and separate the sample chamber and filter housing contents in at least another configuration.

In some embodiments, the plunger or plungers (e.g., in the second chamber) have the dual function of (i) moving to occlude or open a conduit to another chamber, such that the contents of the plurality of chambers can be selectively dispensed (e.g., a valve-like function), and (ii) moving to pressurize and dispense contents of the plurality of chambers. In some embodiments, the movement of the plunger, one or more plungers, or two or more plungers can be described as a moving conduit.

In some embodiments, a portion of the cap protrudes into the sample preparation body, and the portion is configured to advance further into the sample preparation body and operatively connect to the plunger or plungers in the second chamber. In some embodiments, the portion of the cap protruding into the sample preparation body actuates the plunger or plungers.

In some embodiments, the plungers disposed within a second chamber to form a plurality of chambers require that consecutive plungers form a sealed chamber in at least one configuration. In some embodiments, a plunger disposed within a second chamber to form a plurality of chambers require that formation of a sealed chamber between the plunger and a second chamber wall in at least one configuration. In at least one configuration, the sealed chamber is not fluidically connected to another chamber in the device (e.g., the waste chamber 418, the reagent chamber, and/or sample chamber).

In at least one configuration, the plunger or plungers move and the opening (e.g., second chamber opening) configured to fluidically connect the second chamber and the sample chamber (wherein the sample chamber comprises a filter) or a filter housing in at least one configuration is exposed, thereby opening a conduit, and the compression on the plunger or plungers facilitates dispensing the contents of the previously sealed chamber to another chamber (e.g., an adjacent chamber or chamber connected by conduit thereto). See, for example, FIGS. 4, 5, 21-22C. In some embodiments, this differs from some pre-existing reagent dispensing devices that do not have plungers functioning in some configurations to hermetically seal the reagent chambers or pre-existing reagent dispensing devices that already have fluidic connection throughout their chambers.

In some embodiments, with multiple plungers in the second chamber, each plunger movably slides along the length of the second chamber, each progressively occluding then exposing the opening in the second chamber to the contents of one of the plurality of chambers.

In some embodiments, the plungers are configured to move along the length of the second chamber and the opening such that only one of the plurality of chambers in the second can fluidically connect to the sample chamber (wherein the sample chamber comprises a filter) or a filter housing at once.

In some embodiments, the plungers are configured to slidably move along the length of the second chamber such that the contents of one of the plurality of chambers in the second chamber are fully dispensed (or substantially dispensed) into the sample chamber (wherein the sample chamber comprises a filter) or a filter housing before a subsequent chamber of the plurality of chambers fluidically connects to the sample chamber (wherein the sample chamber comprises a filter) or a filter housing through the opening.

In some embodiments, with one plunger in the second chamber, a first inner chamber and second inner chamber are formed in the second chamber. The plunger movably slides along the length of the second chamber. In one configuration, the plunger does not occlude the opening (e.g., the opening is exposed) and the first inner chamber is fluidically connected to the sample chamber (wherein the sample chamber comprises a filter) or a filter housing through the opening, while the second inner chamber is sealed off from the sample chamber (wherein the sample chamber comprises a filter) or a filter housing. In a second configuration, the plunger has moved to occlude the opening, thereby sealing off at least the first inner chamber from the sample chamber (wherein the sample chamber comprises a filter) or a filter housing. In some embodiments, in the second configuration, the plunger fully occludes the opening, and neither the first nor the second chamber are fluidically connected to the sample chamber (wherein the sample chamber comprises a filter) or a filter housing (i.e. both the first and the second chamber are sealed off from the sample chamber (wherein the sample chamber comprises a filter) or a filter housing). In some embodiments, in the second configuration, the plunger partially occludes the opening such that one of the first inner and second inner chambers is fluidically connected to the sample chamber (wherein the sample chamber comprises a filter) or a filter housing, but not both. In a third configuration, the plunger has moved past the opening (e.g., the opening is exposed) and the second inner chamber is fluidically connected to the sample chamber (wherein the sample chamber comprises a filter) or a filter housing through the opening, while the first inner chamber is sealed off from the sample chamber (wherein the sample chamber comprises a filter) or a filter housing.

In some embodiments, with one plunger in the second chamber, a first inner chamber and second inner chamber are formed in the second chamber. The plunger is configured to slidably move in the second chamber and expose and occlude (fully or partially) the opening in the second chamber.

In some embodiments, with one plunger in the second chamber, a first inner chamber and second inner chamber are formed in the second chamber. In some embodiments, the plunger is configured to move along the length of the second chamber and the opening such that only one of the first and second inner chambers can fluidically connect to the sample chamber (wherein the sample chamber comprises a filter) or a filter housing at once.

In one configuration, the plunger does not occlude the opening (e.g., the opening is exposed) and the first inner chamber is fluidically connected to the sample chamber (wherein the sample chamber comprises a filter) or a filter housing through the opening, while the second inner chamber is sealed off from the sample chamber (wherein the sample chamber comprises a filter) or a filter housing. In a second configuration, the plunger has moved to occlude the opening, thereby sealing off at least the first inner chamber from the sample chamber (wherein the sample chamber comprises a filter) or a filter housing. In some embodiments, in the second configuration, the plunger fully occludes the opening, and neither the first nor the second chamber are fluidically connected to the sample chamber (wherein the sample chamber comprises a filter) or a filter housing (i.e., both the first and the second chamber are sealed off from the sample chamber (wherein the sample chamber comprises a filter) or a filter housing). In some embodiments, in the second configuration, the plunger partially occludes the opening such that one of the first inner and second inner chambers is fluidically connected to the sample chamber (wherein the sample chamber comprises a filter) or a filter housing, but not both. In a third configuration, the plunger has moved past the opening (e.g., the opening is exposed) and the second inner chamber is fluidically connected to the sample chamber (wherein the sample chamber comprises a filter) or a filter housing through the opening, while the first inner chamber is sealed off from the sample chamber (wherein the sample chamber comprises a filter) or a filter housing.

In some embodiments, the plunger is configured to slidably move along the length of the second chamber such that the contents of one of the first inner chamber are fully dispensed (or substantially dispensed) into the sample chamber (wherein the sample chamber comprises a filter) or a filter housing before the second inner chamber fluidically connects to the sample chamber (wherein the sample chamber comprises a filter) or a filter housing through the opening.

In some embodiments, “substantially dispensed” refers to dispensing of more than about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, or about 99.9% of contents. In some embodiments, “substantially dispensed” refers to dispensing of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, or about 99.9% of contents. In some embodiments, “substantially dispensed” refers to dispensing of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, or about 99.9% of contents.

In some embodiments, the opening fluidically connecting the second chamber and the sample chamber (wherein the sample chamber comprises a filter) or a filter housing is disposed in the wall of the second chamber. In some embodiments, the opening fluidically connecting the second chamber and the sample chamber (wherein the sample chamber comprises a filter) or a filter housing is disposed in the wall of the sample chamber (wherein the sample chamber comprises a filter) or a filter housing. In some embodiments, there is an opening in the wall of sample chamber (wherein the sample chamber comprises a filter) or a filter housing and another opening in the wall of the second chamber, forming a conduit from the sample chamber (wherein the sample chamber comprises a filter) or a filter housing to the second chamber.

In some embodiments, the second chamber further comprises a breakable barrier, configured to be broken by pressurization from closing of the cap/movement of plungers. Once broken, the breakable barrier fluidically connects the second chamber and the sample chamber (wherein the sample chamber comprises a filter) or a filter housing.

In some embodiments, the sample chamber comprises a first end, a second end, and a filter. In some embodiments, the filter forms one of the first end or the second end of the sample chamber, while the other end is either (i) opened or “uncapped” to allow sample fluid to be introduced or (ii) sealed, covered or capped, e.g., having received the filter plunger by assembly of the cap.

In some embodiments, the filter housing comprises a first end, a second end, and optionally a filter. In some embodiments, the filter forms (or occurs on) one of the first end or the second end of the filter housing. In some embodiments the other end comprises an outlet 426, e.g., to allow filtrate to be dispense from the filter housing e.g., into a chamber (e.g. waste chamber, prepare sample chamber) or an amplification module. In some embodiments, the filter or filtering material is disposed within the filter housing.

In some embodiments, the sample chamber and second chamber are concentric. In some embodiments, the sample chamber is an inner body. In some embodiments, the second chamber is an outer body. In some embodiments, the inner body is disposed within the outer body. In some embodiments, the sample chamber and second chamber are disposed within the sample preparation body and adjacent to one another. In some embodiments, the filter housing and second chamber are concentric. In some embodiments, the filter housing and sample chamber are concentric. In some embodiments, the filter housing and the second chamber are adjacent to one another. In some embodiments, the filter housing and sample chamber are adjacent to one another.

In some embodiments, the sample preparation device comprises: (a) a sample preparation body comprising an inner body, an outer body, a waste chamber, and a prepared sample chamber 420, wherein the inner body comprises a sample chamber for holding sample fluid, wherein the prepared sample chamber comprises (i) a breakable barrier 422 or valve and (ii) an outlet port 424, wherein the sample chamber comprises (i) an first end, (ii) a filter, and (iii) a second end, wherein second end of the sample chamber comprises an outlet, optionally a tapered outlet, optionally a hollow piercing member, configured to allow filtrate flow therethrough, wherein the outer body comprises one plunger, one or more plungers, or two or more plungers disposed therein to form a plurality of chambers, wherein the plurality of chambers comprise at least one of reagent, aqueous fluid, or air. In some embodiments, the outer body comprises an opening fluidically connecting the outer body to the filter and second end of the sample chamber. In some embodiments, the inner body comprises an opening fluidically connecting the outer body to the filter and second end of the sample chamber.

In some embodiments, the sample preparation device comprises: sample preparation system 100 for processing biological samples for testing for a disease or condition, the system comprising: (a) a sample preparation body comprising an inner body, an outer body, a filter housing, a waste chamber, and a reagent chamber. In some embodiments, the inner body comprises a sample chamber for holding sample fluid. In some embodiments, the filter housing is fluidically connected to the sample chamber. In some embodiments, the waste chamber comprises a prepared sample chamber. In some embodiments, the prepared sample chamber comprises (i) a breakable barrier or a valve sealing it from the waste chamber and (ii) an outlet port. In some embodiments, the filter housing comprises (i) an first end, (ii) a filter, and (iii) a second end, wherein the second end comprises an outlet configured to allow filtrate flow therethrough. In some embodiments, the outlet is a tapered outlet. In some embodiments, the outlet is a hollow piercing member.

In some embodiments, the filter housing is the filter.

In some embodiments, the outer body comprises one or more plungers disposed therein to form at least one aqueous fluid chamber and/or at least one air chamber.

Additionally, in some embodiments, the sample preparation system comprises: (b) a cap (e.g., a screw cap) configured for mounting on the open end of the sample preparation body. In some embodiments, the cap comprises a filter plunger aligned to enter the open end of the sample chamber when the sample preparation body is closed by the assembly of the cap to the sample preparation body.

In some embodiments, the prepared sample chamber is a chamber that receives lysate or is configured to receive lysate, e.g. via the outlet of the filter housing.

In some embodiments, the sample preparation device has a reception configuration and a preparation configuration, the device being movable from the reception configuration, in which the sample fluid can be introduced into the sample chamber, to the preparation configuration, in which the sample preparation body is closed by the assembly of the cap (e.g., screw cap) to the sample preparation body. In some embodiments, in the reception configuration, the opening is occluded by the plunger or one of the two or more plungers, e.g., wherein in the preparation configuration, the device is actuated by a user closing the cap (e.g., screwing on the screw cap), thereby compressing the filter plunger against the filter, driving the sample fluid therethrough and into the waste chamber, and wherein further closing of the cap (e.g., screwing of the cap) moves the plunger or the two or more plungers in the outer body, and as the plunger or each of the two or more plungers move, they expose and occlude the opening to selectively dispense contents of the plurality of chambers through the filter and second end of the sample chamber (wherein the sample chamber comprises a filter) or a filter housing.

In some embodiments, the present disclosure provides a sample preparation device as described herein and an amplification module operatively couplable to the sample preparation body and adjacent to the prepared sample chamber. In some embodiments, the amplification module comprises an interface, wherein the outlet port of the prepared sample chamber is configured to receive the interface, thereby fluidically connecting the prepared sample chamber (wherein the sample chamber comprises a filter) or a filter housing and the amplification module. In some embodiments, the sample preparation device has a reagent chamber. In some embodiments, the amplification module has a reagent plunger configured to move within the reagent chamber.

In some embodiments, the present disclosure provides a system for processing biological samples for testing for a disease or condition, the system comprising: (a) a sample preparation body comprising an inner body, an outer body, a waste chamber, a reagent chamber, and optionally a prepared sample chamber. In some embodiments, the present disclosure provides a system for processing biological samples for testing for a disease or condition, the system comprising: (a) a sample preparation body comprising an inner body, an outer body, a filter housing, a waste chamber, a reagent chamber, and optionally a prepared sample chamber. In some embodiments, rather than having a prepared sample chamber, the filter housing comprises an outlet configured to fluidically connect directly to the amplification module, e.g., after linear actuation of a chamber or plate or part of the system; e.g., after a breakable membrane on the amplification module or the outlet is broken; e.g. after a valve on the amplification module or the outlet is actuated.

In some embodiments, the inner body comprises a sample chamber for holding sample fluid, optionally wherein the waste chamber comprises the prepared sample chamber, wherein the prepared sample chamber comprises (i) a breakable barrier or valve sealing it from the waste chamber and (ii) an outlet port, wherein the sample chamber comprises (i) a first end, (ii) a filter, and (iii) a second end, wherein the second end of the sample chamber comprises an outlet (optionally a tapered outlet, optionally a piercing member, e.g., tapered piercing member) configured to allow filtrate flow therethrough.

Alternatively, in some embodiments, there is a sample chamber for holding sample fluid, optionally wherein the waste chamber comprises the prepared sample chamber, wherein the prepared sample chamber comprises (i) a breakable barrier or valve sealing it from the waste chamber and (ii) an outlet port, wherein the filter housing comprises (i) a first end and (iii) a second end, and optionally (iii) a filter disposed within, wherein the second end of the sample chamber comprises an outlet (optionally a tapered outlet, optionally a piercing member, e.g., tapered piercing member) configured to allow filtrate flow therethrough. In some embodiments, the outer body comprises a plunger or two or more plungers disposed therein to form a plurality of chambers, e.g., at least one aqueous fluid chamber and at least one air chamber.

In some embodiments, the system further comprises an amplification module operatively coupled to the sample preparation body and adjacent to the prepared sample chamber. In some embodiments, the amplification module comprises an interface and a reagent plunger configured to move within the reagent chamber. In some embodiments, the outlet port of the prepared sample chamber is configured to receive the interface, thereby fluidically connecting the prepared sample chamber and the amplification module, and wherein the reagent plunger is configured to move within the reagent chamber.

some embodiments, the system further comprises a cap configured for mounting on the open end of the sample preparation body, wherein the cap comprises a filter plunger aligned to enter the open end of the sample chamber when the sample preparation body is closed by the assembly of the cap to the sample preparation body.

In some embodiments, the amplification module comprises a piercing member. In some embodiments, the amplification module comprises the piercing member rather than the sample chamber outlet having a piercing member. In some embodiments, the prepared sample chamber further comprises a breakable barrier sealing it from the amplification module.

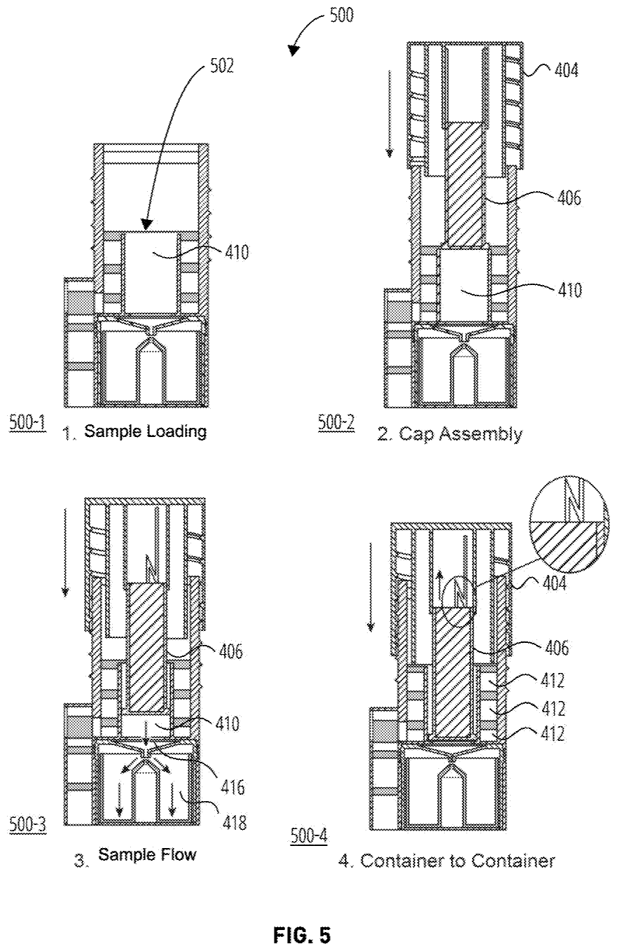

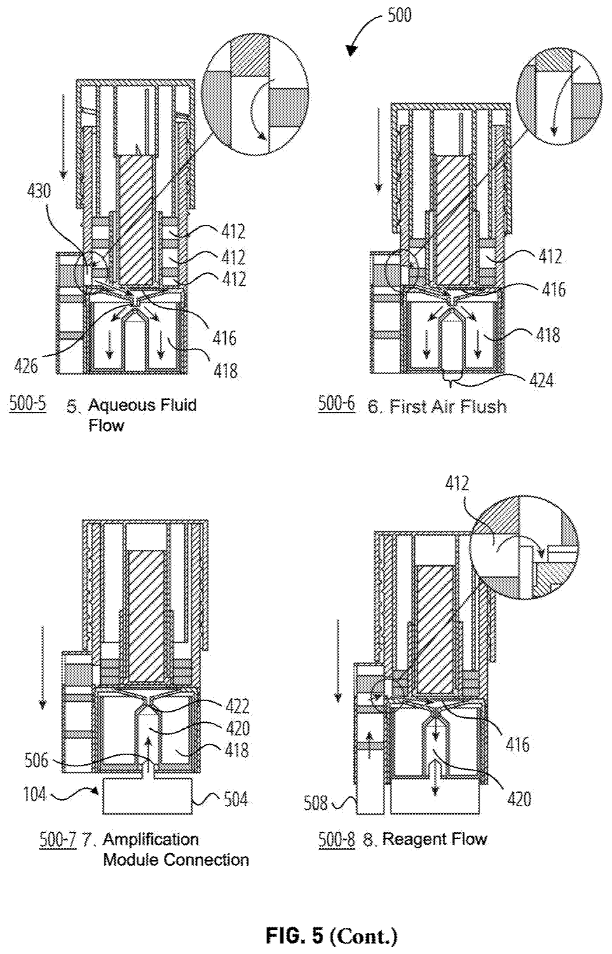

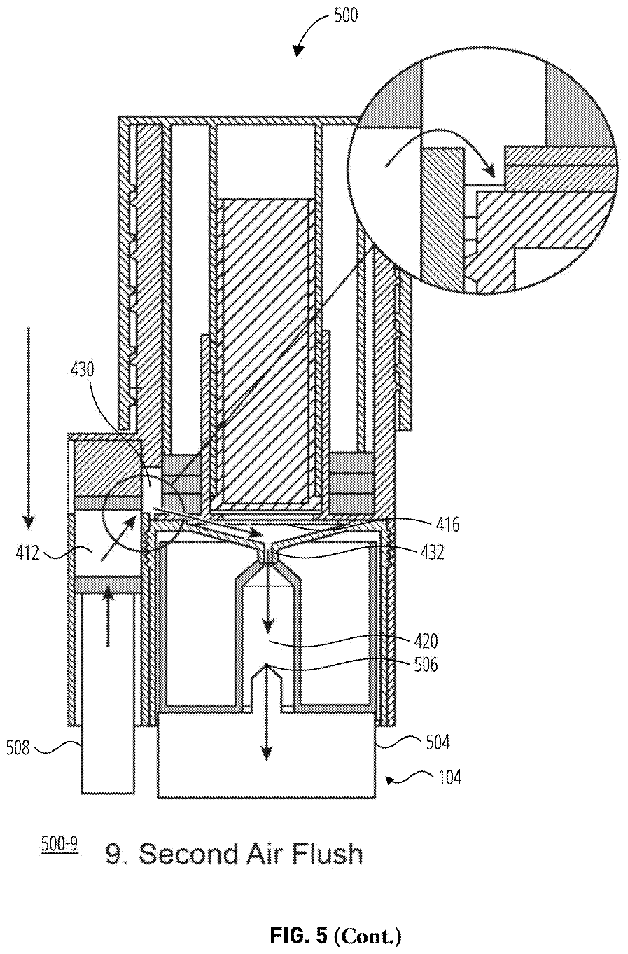

In some embodiments, the sample preparation system has a reception configuration, a filtering configuration, and a reagent configuration, the system being movable from the reception configuration in which the sample can be introduced into the sample chamber, to the filtering configuration in which sample preparation body is closed by the assembly of the cap to the sample preparation body, to the reagent configuration, wherein in the filtering configuration, the filter plunger pressurizes the sample chamber, thereby driving the sample fluid through the filter or filter housing and into the waste chamber, and wherein further compressing the cap against the sample preparation body moves the two or more plungers to open and close and opening in the outer body so as to selectively dispense fluid (e.g., contents of the water and air chambers) through the filter or filter housing and waste chamber, wherein in the reagent configuration, further compressing the cap against the sample preparation body operatively couples the second end and prepared sample chamber, thereby breaking the breakable barrier or opening the valve and fluidically connecting the reagent chamber, the filter or filter housing, and the prepared sample chamber, and further compressing the cap moves the reagent plunger into the reagent chamber, thereby driving the contents of the reagent chamber through the filter or filter housing to the prepared sample chamber. See, for example, FIG. 5.

At step 500-1, a sample fluid 502 may be loaded into the sample chamber 410. A user may manually load the sample chamber 410 with the sample fluid 502. In some embodiments, about 5 mL of sample fluid 502 may be loaded into the sample chamber 410. The sample fluid 502 may include any sample described herein. As a non-limiting example, the sample fluid 502 may be urine.

At step 500-2, a user may rotate or screw the cap 404 of the sample preparation device 102. A rotation of the cap 404 of the sample preparation device 102 may align the filter plunger 406 of the sample preparation device 102 into the sample chamber 410.

At step 500-3, the sample fluid 502 may flow through the filter housing 416 and into the waste chamber 418 as the filter plunger 406 moves downwards.

At step 500-4, as the filter plunger 406 bottoms out, the cap 404 may slip past a mechanical interface such that its travel can continue. A travelling of the cap 404 may allow for a compressing of the aqueous fluid chamber 412 and/or the first air chamber 412 of the sample preparation device 102.

At step 500-5, the first air chamber 412 may be compressed. A compression of the first air chamber 412 and an incompressibility of the aqueous fluid chamber 412 may drive the filter plunger 406 down, which may open an outlet sealed by a breakable seal 422 for water of the aqueous fluid chamber 412. Water may flow through the filter housing 416 and into the waste chamber 418.

At step 500-6, once the water has flowed through the filter housing 416, an outlet may be opened for the first air chamber 412. Air may be flushed through the filter housing 416 from the first air chamber 412.

At step 500-7, a user may push the sample preparation device 102 onto an amplification module interface 504. A connection of the sample preparation device 102 with the amplification module interface 504 may drive the waste chamber 418 and/or prepared sample chamber 420 upwards, which may pierce a breakable seal 422 of the sample preparation device 102 and re-direct any fluid flow into the prepared sample chamber 420. The prepared sample chamber 420 may house a resuspension buffer.

At step 500-8, a reagent plunger 508 on an amplification module may drive the plungers 414 in the reagent chamber 428, for lysis and/or air, upwards. As the reagent plunger 508 moves, an outlet may be opened to pass the lysis through the filter housing 416 and into the prepared sample chamber 420 for resuspension and into an amplification module.

At step 500-9, as the reagent chamber 428 is compressed, the outlet to the second air chamber may be opened which may flush air though the filter housing 416 into the prepared sample chamber 420 and an amplification module.

In some embodiments, a plurality of chambers comprise one or more of the following: a rinse chamber, an inner reagent chamber, and an air chamber. In some embodiments, the plurality of chambers comprises one or more of an inner reagent chamber and an air chamber. In some embodiments, the plurality of chambers comprises one or more of a rinse chamber and an air chamber. As used herein, a “rinse chamber” refers to a chamber having aqueous fluid (e.g. water) and/or a hydration buffer. The rinse chamber may also have a diluting liquid. In some embodiments, the rinse chamber is useful for providing a water (or buffer) flush, which can be useful directly after a conduit/chamber receives a reagent or sample fluid (during filtering). As used herein, an “inner reagent chamber” refers to a chamber having a reagent as defined herein. As used herein, an “air chamber” refers to a chamber having air and that is useful for providing an air flush.

In some embodiments, for a aqueous fluid flush or equivalent, as the user compresses the cap (e.g., via screwing), the contents of the rinse chamber are dispensed immediately after driving the sample fluid through the filter. In some embodiments, as the user compresses the cap (e.g., via screwing), the contents of the first inner reagent chamber are dispensed immediately after the contents of the rinse chamber are driven through the filter. In some embodiments, for an air flush, as the user compresses the cap (e.g., via screwing), the contents of the air chamber are dispensed immediately after the contents of the rinse chamber are driven through the filter. In some embodiments, as the user compresses the cap (e.g., via screwing), the contents of the first inner reagent chamber are dispensed immediately after the contents of the air chamber are driven through the filter and prepared sample chamber.

In some embodiments, the plurality of chambers further comprise multiple inner reagent chambers, and optionally, the multiple inner reagent chambers are configured to be sequentially dispensed. In some embodiments, each inner reagent chamber is dispensed immediately after a rinse chamber is dispensed. In some embodiments, an air chamber is dispensed immediately after a rinse chamber is dispensed.

In some embodiments, the present disclosure provides a sample preparation device for processing biological samples for testing for a disease or condition, the device comprising: a sample preparation body comprising (i) a sample chamber for holding sample fluid (ii) a filter housing that comprises a filter and is fluidically connected to the sample chamber; (iii) a second chamber comprising one or more plungers disposed therein to form a plurality of chambers. In some embodiments, the plurality of chambers each comprise at least one of a reagent, aqueous fluid, or air. In some embodiments, the second chamber comprises an opening fluidically connecting the filter housing and the second chamber, wherein the opening is configured to be opened and closed upon movement of the plunger through the second chamber so as to selectively dispense contents of the plurality of chambers into the filter housing (e.g., through the filter). In some embodiments, the sample preparation device further comprises a cap comprising a filter plunger, wherein the cap is configured for mounting on an open end of the sample preparation body, wherein the filter plunger is configured to compress the sample fluid against the filter or filter housing. In some embodiments, the sample preparation device further comprises a reagent chamber comprising one or more plungers disposed therein to form a plurality of chambers, wherein the plurality of chambers each comprise at least one of a reagent, aqueous fluid, and air. In some embodiments, the reagent chamber is configured to receive waste in at least one configuration.

In some embodiments, the second chamber's plurality of chambers each comprise one of aqueous fluid and air. In some embodiments, the second chamber's plurality of chambers each comprise air. In some embodiments, the reagent chamber's plurality of chambers each comprise one of reagent or air or is configured to receive waste. In some embodiments, at least one of the reagent chamber's plurality of chambers is configured to receive waste.

In some embodiments, one end of the reagent chamber is adjacent to the sample chamber, and a second end of the sample chamber is sealed by a movable plate (also referred to as bottom plate) 2202 configured to move the reagent chamber plunger(s) by pressurization or physical contact with a plunger.

In some embodiments, the sample chamber comprises an outlet (optionally a tapered outlet, optionally a piercing member, e.g., tapered piercing member) configured to allow filtrate flow therethrough. In some embodiments, the filter housing comprises an outlet (optionally a tapered outlet, optionally a piercing member, e.g., tapered piercing member) configured to allow filtrate flow therethrough. In one configuration the outlet is open (and, e.g., suspended in the waste chamber), thereby allowing filtrate to flow from the sample chamber (wherein the sample chamber comprises a filter) or a filter housing to the waste chamber. In another configuration, the outlet contacts or receives an interface, thereby fluidically connecting the sample chamber (wherein the sample chamber comprises a filter) or a filter housing and the amplification module. In some embodiments, in such configuration, the movable plate comprises a breakable membrane that is broken upon contact with the outlet, thereby creating the fluid connection between the sample chamber and amplification module. In some embodiments, the waste chamber is sealed off from the sample chamber and the amplification module.

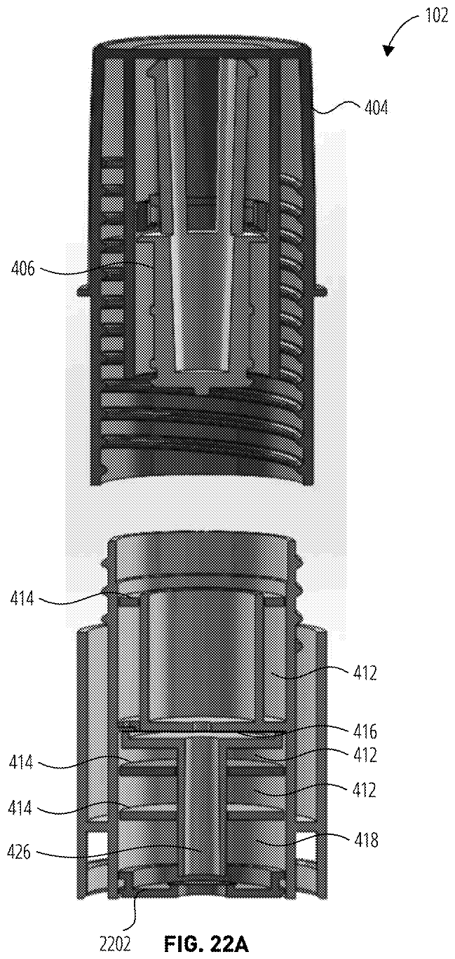

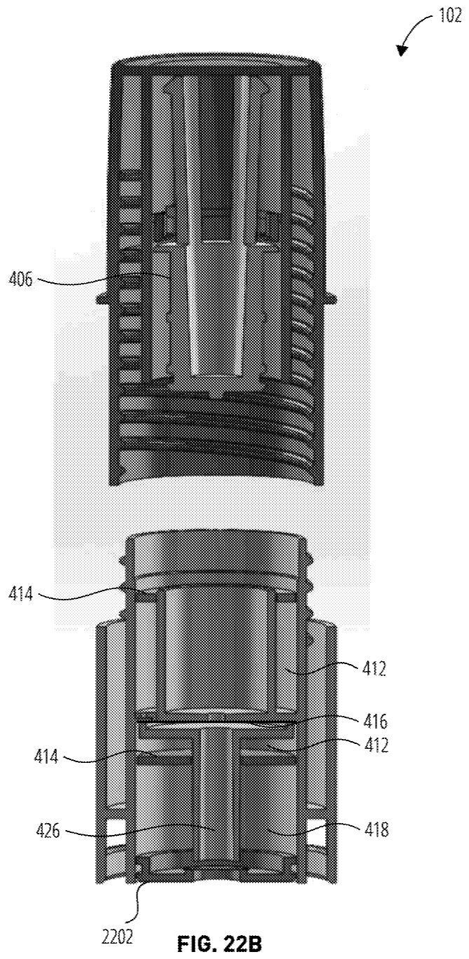

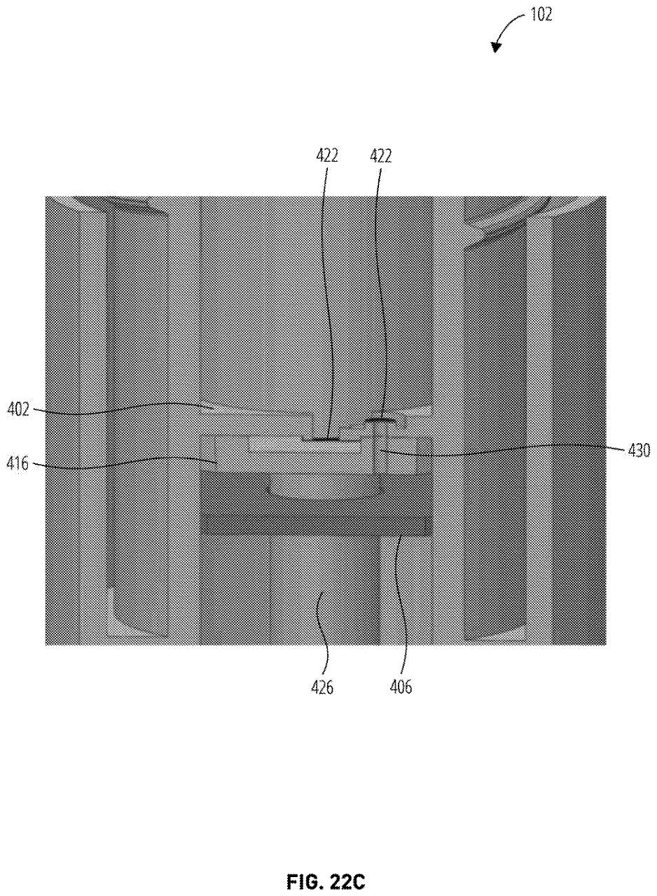

In some embodiments, the reagent chamber comprises an opening configured to be opened and closed (e.g., exposed and covered, exposed and occluded, etc.) upon movement of the plunger(s) in the reagent chamber. In one configuration, the opening fluidically connects the sample chamber (wherein the sample chamber comprises a filter) or a filter housing and one of the plurality of chambers in the reagent chamber (not a waste chamber, e.g., an inner reagent chamber). In some embodiments, this is after the fluidic connection of the sample chamber (wherein the sample chamber comprises a filter) or a filter housing and the amplification module. In such configuration, upon movement of the one or more plungers in the reagent chamber, the opening is exposed to selectively dispense contents of one of the plurality of chambers (e.g., reagent, e.g., lysate) into the sample chamber (wherein the sample chamber comprises a filter) or a filter housing, through the filter, and into the amplification module. See, e.g., FIGS. 22A-22C.

In some embodiments, rather than an opening in the reagent chamber, a portion of the wall of the reagent chamber comprises a breakable barrier, configured to be broken by closing of the cap, or progressive closing of the cap, and the internal pressurization from the cap closure.

In some embodiments, the reagent chamber only has one plunger disposed therein to form an inner reagent chamber and a waste chamber. See, e.g., FIGS. 22B and 22C.

In some embodiments, the sample fluid, aqueous fluid chamber(s) and air chamber(s), e.g., the first air flush chamber, are stored independently upstream (e.g., above) the filter. As the user compresses the cap, the filter plunger first pushes the sample fluid through the filter before the two or more plungers push water and the first air flush chase through the filter to the waste chamber.

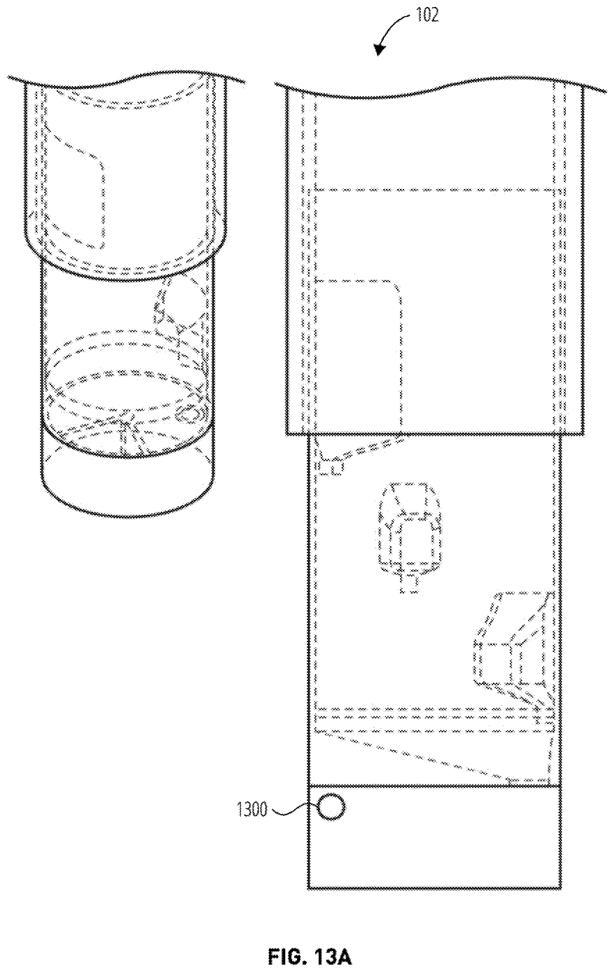

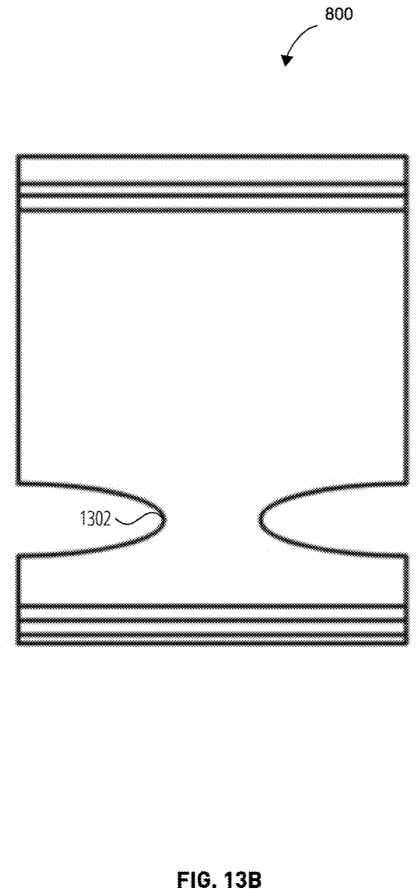

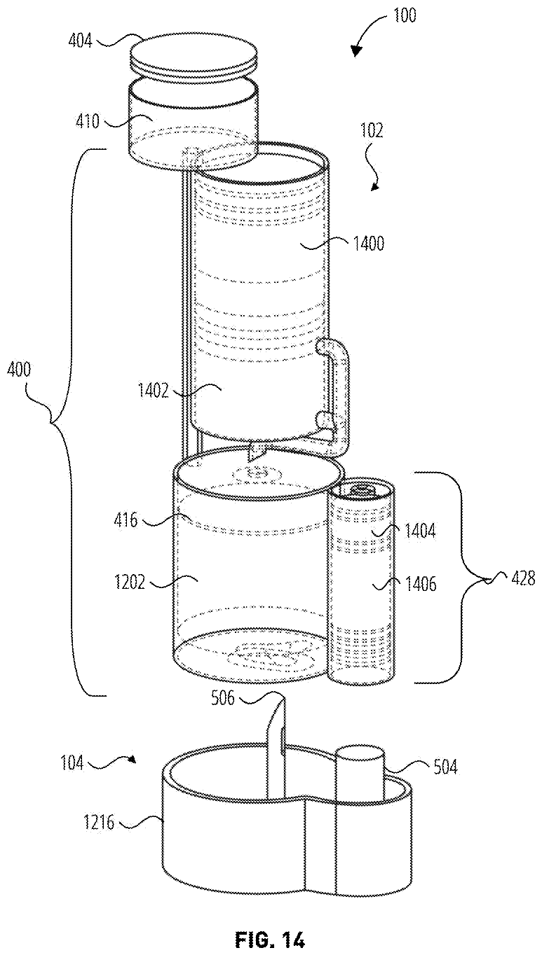

In some embodiments, a waste chamber can have a vent hole 1300 to normalize air pressure (e.g. FIG. 13A).

On assembly to the amplification cassette, the downstream chamber is swapped from waste to lysate and the lysis and second air flush are pushed through the system.

In some embodiments, the outer body comprises 1, 2, 3, 4, 5, 6, 7, or more plungers, forming a plurality of chambers therein. In some embodiments, the sample preparation body comprises another chamber comprising a plurality of plungers, forming a plurality of chambers, wherein each of the plurality of chamber comprises at least one of aqueous fluid, reagent, and air.

In some embodiments, the amplification module comprises one or more reaction chambers. In some embodiments, the amplification module comprises one or more valves or breakable barriers. In some embodiments, the amplification module comprises a display for the test results. The display can offer a simple read out for the display (e.g., binary readout indicating positive or negative detection). Examples of readouts contemplated for the assemblies disclosed herein include LCD, LED, lateral flow strip, colorimetric detection (e.g., with colorimetric dyes), halochromic detection, or mobile connectivity. Additionally, the amplification module can comprise one or more of a heater, a filter, additional reagent for the initiation of the assay, an amplification mixture (to enable nucleic acid amplification), microfluidic channels, microfluidic pumps, and one or more batteries 200.

In some embodiments, the sample preparation device and/or amplification module weigh less than 1 pound. In some embodiments, the sample preparation device and/or amplification module weigh less than about 0.5 lb., about 0.6 lb., about 0.7 lb., about 0.8 lb., about 0.9 lb., about 1 lb., about 1.1 lb., about 1.2 lb., about 1.3 lb., about 1.4 lb., about 1.5 lb., about 1.6 lb., about 1.7 lb., about 1.8 lb., about 1.9 lb., or about 2.0 lb. In some embodiments, all linear dimensions of the sample preparation device and/or amplification module are less than 5 inches in length. In some embodiments, all linear dimensions of the sample preparation device and/or amplification module are less than about 4 in., about 4.1 in., about 4.2 in., about 4.3 in., about 4.4 in., about 4.5 in., about 4.6 in., about 4.7 in., about 4.8 in., about 4.9 in., about 5 in., about 5.1 in., about 5.2 in., about 5.3 in., about 5.4 in., about 5.5 in., about 5.6 in., about 5.7 in., about 5.8 in., about 5.9 in., or about 6 in. in length.

In some embodiments, the sample preparation systems disclosed herein comprise lyophilized reagents. In some embodiments, the lyophilized reagents are in the sample preparation systems. In some embodiments, the sample preparation systems have a long shelf-life. For example, some of the assay assemblies have a shelf life of 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months.

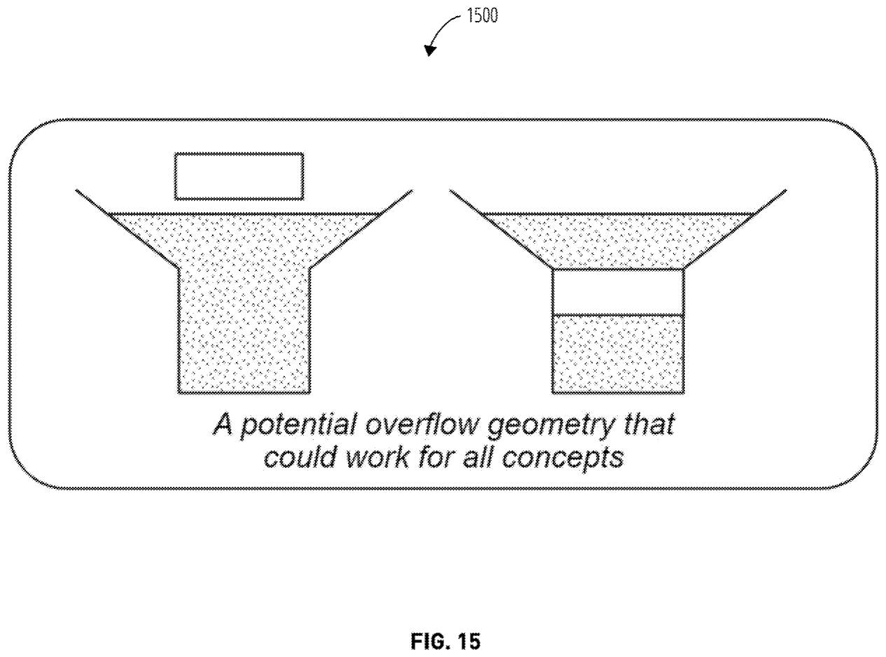

In some embodiments, the sample chamber comprises one or more fill lines 408, which guide the user in filling the sample chamber in the reception configuration. In alternate embodiments, the sample chamber is configured with an overflow prevention system. For example, the sample chamber widens proximal to the user and the diameter of the filter plunger at initial engagement matches the required fill level for the sample chamber. As a result, a user can overfill the sample chamber but once the filter plunger initially engages within the sample chamber, only the required volume of sample fluid is captured between the filter plunger and the filter. See, for example, FIG. 15.

In some embodiments, the pressure applied on fluids (e.g., gases and/or liquids) within a chamber of a disclosed sample preparation device is dependent on the speed of the component coupling (e.g., compression of the cap against the sample preparation body) and/or type of filter used. The pressure (e.g., differential pressure) generated within one or more parts of the device during actuation can be measured using a pressure sensor device. In some embodiments, the maximum pressure generated is no more than about 10 bar. In some embodiments, the maximum pressure generated is no more than about 9 bar. In some embodiments, the maximum pressure generated is no more than about 8 bar. In some embodiments, the maximum pressure generated is no more than about 7 bar. In some embodiments, the maximum pressure generated is no more than about 6 bar. In some embodiments, the maximum pressure generated is no more than about 5 bar. In some embodiments, the maximum pressure generated is no more than about 4 bar. In some embodiments, the maximum pressure generated is about 1 bar, 1.5 bar, 2 bar, 2.5 bar, 3 bar, 3.5 bar, 4 bar, 4.5 bar, 5 bar, 5.5 bar, 6 bar, 6.5 bar, 7 bar, 7.5 bar, 8 bar, 8.5 bar, 9 bar, 9.5 bar, or 10 bar. In some embodiments, the maximum pressure generated is about 1.5 bar. In some embodiments, the maximum pressure generated is about 2 bar. In some embodiments, the maximum pressure generated is about 3 bar. In some embodiments, the maximum pressure generated is about 4 bar. In some embodiments, the maximum pressure generated is about 5 bar. In some embodiments, the pressure generated is about 1-5 bar, 2-5 bar, 3-5 bar, 1-6 bar, 2-6, 3-6 bar, 1-7 bar, 2-7 bar, 3-7 bar, or 4-7 bar. In some embodiments, the maximum pressure generated is less than about 1 bar.

The operative coupling of the sample preparation device and amplification module is contemplated using any one of the engagement means disclosed herein, provided it is irreversible or minimizes the risk of the system components decoupling during sample preparation, filtering, lysing, and/or amplification. In some embodiments, the components of the sample preparation system (e.g., sample preparation device, body, cap, and/or amplification module) comprise, e.g., shelves, shelves with gaps, hooks, notches, snap features, cantilever snap features, detents, annular snap features, torsion snap features, slide locks, push locks, key features, twist features, etc. In some embodiments, these engagement means have radial symmetry in their placement on the sample preparation device, body, cap and/or amplification module. The features that provide irreversible coupling or minimize risk of decoupling reduces indeterminate or erroneous test results and reduces the risk of interrupting or terminating the reaction, once the reaction has been initiated in the amplification module or cap assembly to the sample preparation device has been initiated.

In some embodiments, a sample preparation device comprises one or more cantilever snaps that facilitate irreversible coupling of the sample preparation device to the amplification module.

In various embodiments, the sample preparation system, including the sample preparation body and the amplification module, comprises one or more materials including, for example, medical plastics, polymeric materials (e.g., materials having one or more polymers including, for example, plastic and/or rubber), synthetic polymer (e.g., thermoplastics), glass, and/or metallic materials. Materials of which any of the assembly can be composed include, but are not limited to: polymeric materials, e.g., elastomeric rubbers, such as natural rubber, silicone rubber, ethylene-vinyl rubber, nitrile rubber, ethylene propylene diene monomer (EPDM) rubber, butyl rubber; plastics, such as polytetrafluoroethene or polytetrafluoroethylene (PFTE), including expanded polytetrafluoroethylene (e-PFTE), polyethylene, polyester (Dacron™), nylon, polystyrene, polycarbonate, polypropylene, polyethylene, polyallomer, polycarbonate, polycarbonate/acrylonitrile butadiene styrene (PC/ABS), acrylonitrile butadiene styrene (ABS), cycloolefins, cycloolefin copolymers, high-density polyethylene (HDPE), polyurethane, polydimethylsiloxane (PDMS); adhesives, such as acrylic adhesive, silicone adhesive, epoxy adhesive, or any combination thereof; metals and metal alloys, e.g., titanium, chromium, aluminum, stainless steel, and/or glass.

The amplification module can be made of suitable material, such as glass, ceramics, metals, paper, pressed cardboard, or polymers (e.g., synthetic polymers, e.g., thermoplastics), but preferably comprises a plastic, polymer or copolymer such as those that are resistant to breakage. Non-limiting examples of such materials include polystyrene, polypropylene, polyethylene, polyallomer, polycarbonate, polycarbonate/acrylonitrile butadiene styrene (PC/ABS), acrylonitrile butadiene styrene (ABS), cycloolefins, cycloolefin copolymers, ethylene propylene diene monomer (EPDM) rubber, and silicone rubber. In certain embodiments, the amplification module material comprises polystyrene. The materials contemplated for the amplification module are affordable materials, making the assembly disposable and affordable to manufacture in bulk.

In some embodiments, the components of the sample preparation system can be manufactured by milling, injection molding, and/or other suitable methods, as would be known to those of ordinary skill in the art.

According to some aspects, the subject devices and components thereof are hand-held devices. As used herein, the term “hand-held” refers to the characteristic ability of an aspect to be held (e.g., retained, or easily or comfortably held) in a hand, such as the hand of a mammal, such as the hand of a human, such as the hand of an adult male or female human of an average size and/or strength. As such, a hand-held aspect is an aspect that is sized and/or shaped to be retained (e.g., easily or comfortably retained) in the hand of a human. A hand-held aspect can also be an aspect that can be moved (e.g., easily moved, such as easily moved in a vertical and/or horizontal direction) by a human (e.g., one or two hands of a human).

In some embodiments, the methods disclosed herein can also be used for detecting an analyte (any target nucleic acid, e.g., DNA or RNA).

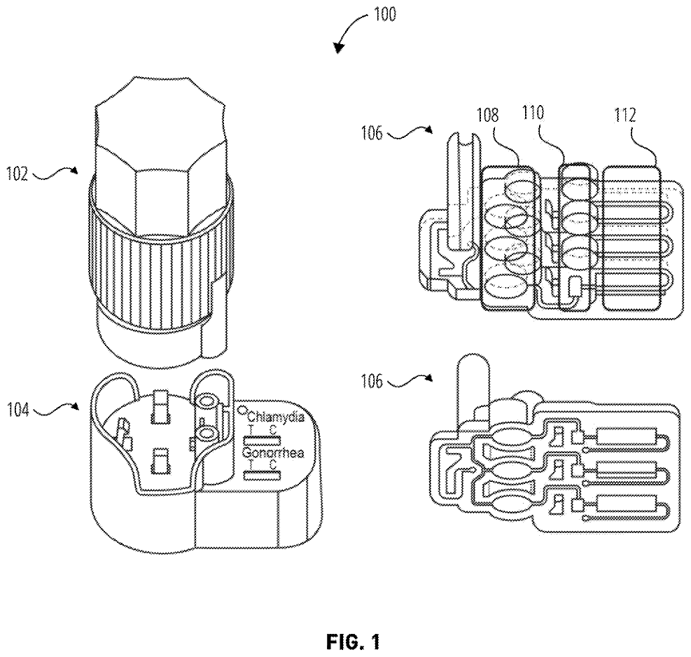

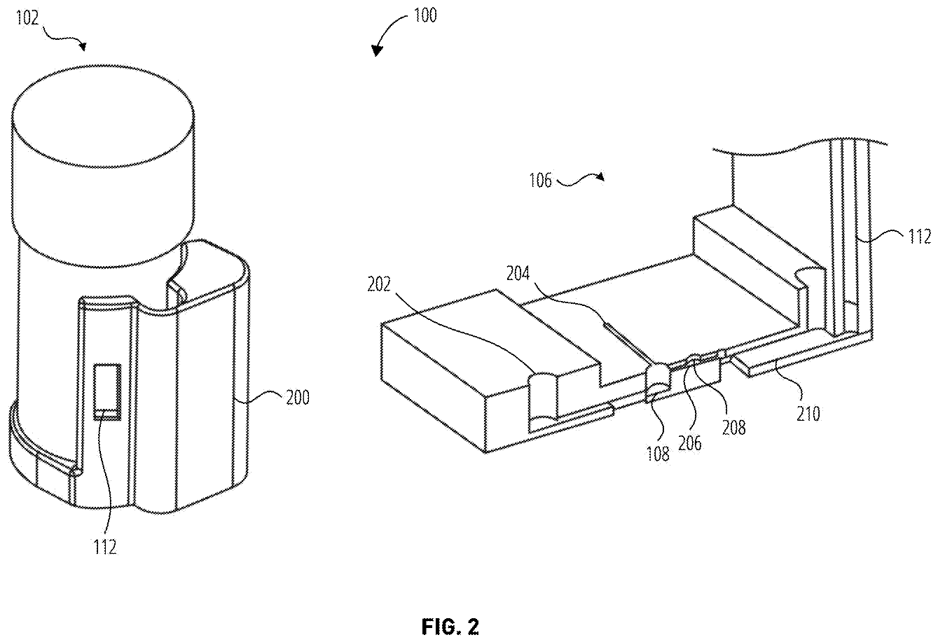

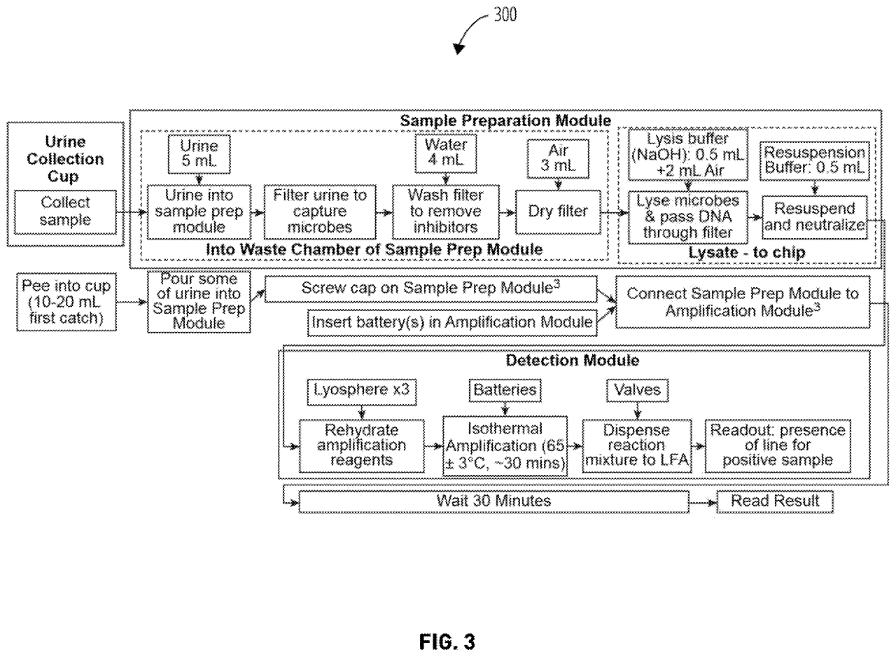

In some embodiments, an amplification module 104 provided herein comprises one or more of the following: a fluidic chip 106, reaction wells 108, valves 110, one or more lateral flow strips 112. See, e.g., FIG. 1. In some embodiments, a fluidic chip comprises one or more of the following: a fluid inlet port 202, an air outlet channel 204, a reaction well 108, fluid outlet channel 206, plug chamber 208, and a reservoir 210.

### II.A. Alternative Device Designs

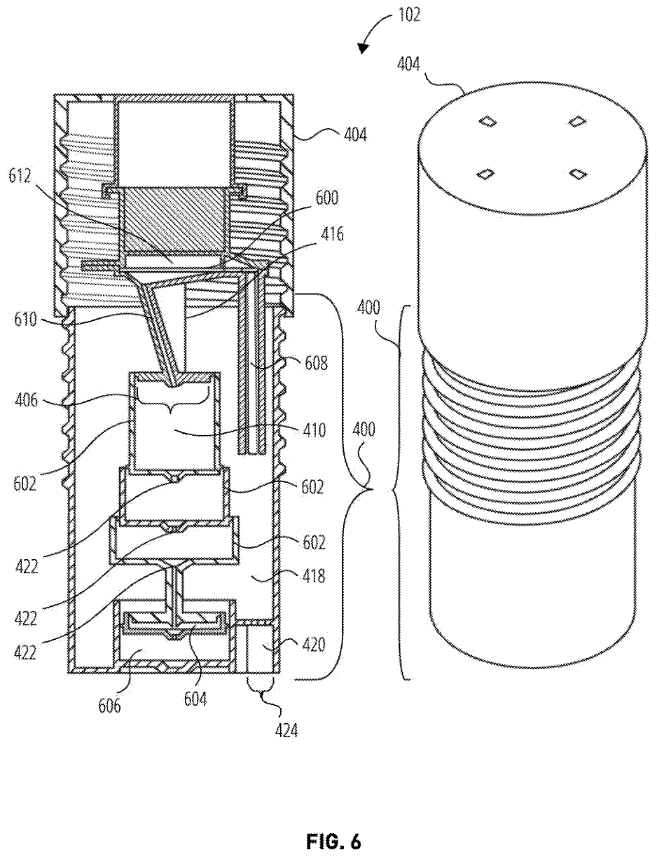

In some embodiments, a sample preparation device of the present disclosure utilizes a stacked-cups configuration, an example of which is shown in FIG. 6. Briefly, a sample preparation body comprises a plurality of stacked cups, each comprising at least one of sample fluid, aqueous fluid, reagent, or air. A filter 600 is disposed within the sample preparation body or within a cap 404. The sample fluid, aqueous fluid, air flush(s), or reagents are all stored in independent cups 602 below the filter 600. The cap comprises a filter plunger 406, which comprises a plunger channel 610 to a filter and another chamber for the filtrate 612. As the user compresses (e.g., screws) the cap, the filter plunger 406 first drives the sample fluid through the plunger channel, and then each preceding cup acts as a plunger to push the liquid up through the filter and through the conduit or channel into another chamber (e.g., a prepared sample chamber). The sample preparation body may be connected to an amplification module.

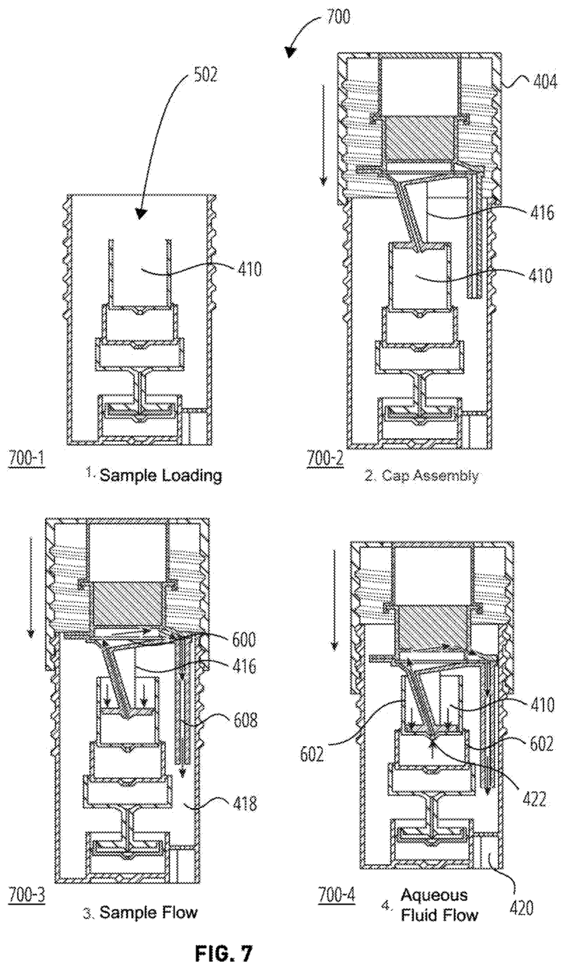

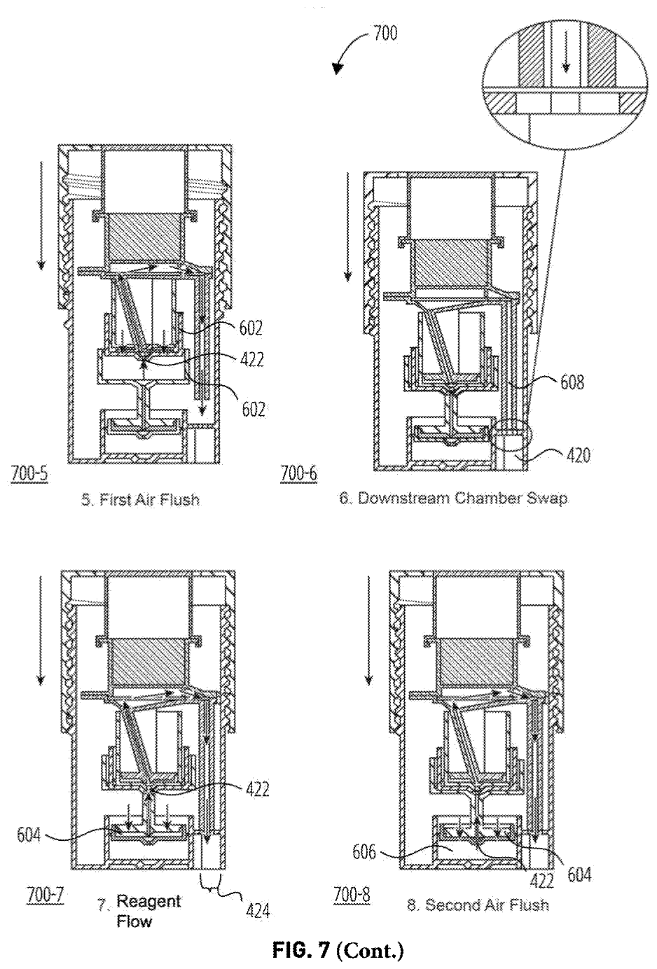

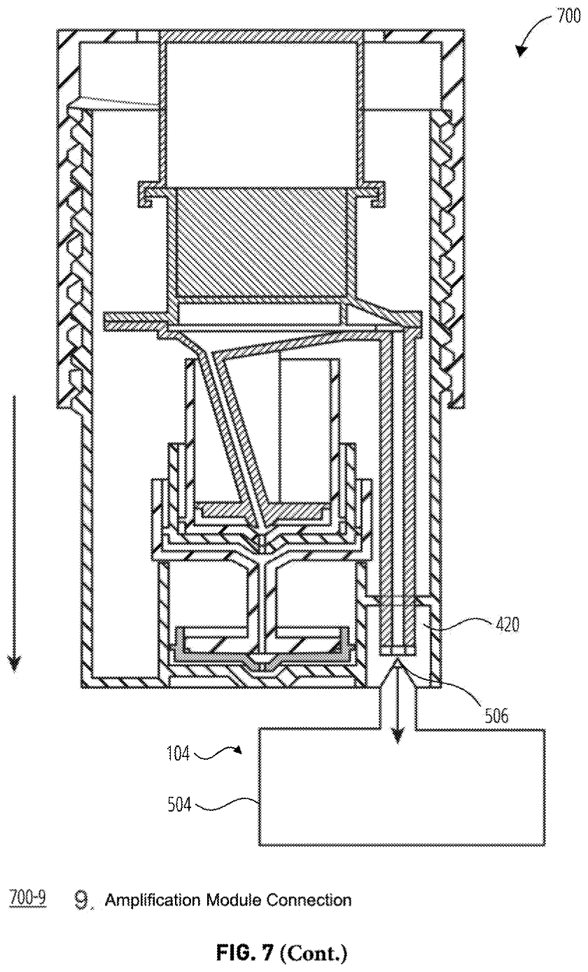

Reference is now made to FIG. 7, which illustrates a workflow of the sample preparation device described above with reference to FIG. 6.

At step 700-1, a sample (interchangeably referred to as “sample fluid”) 502 may be loaded into the sample chamber 410. The sample may be loaded manually by a user. In some embodiments, about 5 mL of the sample 502 may be loaded into the sample chamber.

At step 700-2, a user may screw the cap 404 of the sample preparation device 102, which may align the filter plunger 406 of the sample preparation device into the sample chamber.

At step 700-3, as the filter housing 416 travels down, the sample may be forced up through the filter 600 of the sample preparation device 102 and around an outlet spigot 608 into a waste chamber 418.

At step 700-4, as the filter housing bottoms out, a first breakable seal 422 may burst. The sample chamber 410 may act as a plunger to drive an aqueous fluid up through the filter and around the outlet spigot 608 and into the waste chamber.

At step 700-5, as the sample chamber bottoms out, a second breakable seal 422 is burst. The aqueous fluid chamber may act as a plunger to drive a first air flush up through the filter, around the outlet spigot, and into the waste chamber.

At step 700-6, as an air flush is complete, the outlet spigot may pierce a third breakable seal such that all following fluid may be directed into reagent chamber 604. The reagent chamber may house a resuspension buffer.

At step 700-7, as the aqueous fluid chamber bottoms out, a fourth breakable seal may be burst. The first air chamber 606 may act as a plunger to drive reagent up through the filter, around the outlet spigot and into the prepared sample chamber 420.

At step 700-8, as the first air chamber bottoms outs, breakable seal may burst. The reagent chamber may act as a plunger to drive an air flush up through the filter, around the outlet spigot, and into the prepared sample chamber 420.

At step 700-9, a user may connect the sample preparation device 102 to an amplification module. An interface 506 may pierce or break an underside of the prepared sample chamber 420 to allow flow of the lysate into an amplification module.

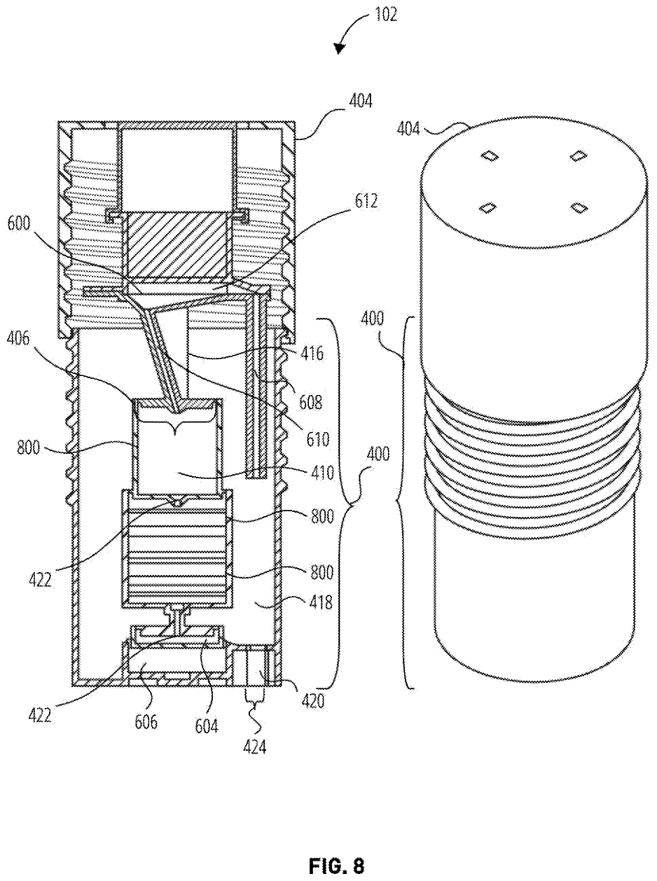

In some embodiments, a sample preparation device of the present disclosure utilizes bags 800, packs, or a deformable receptacle equivalent (e.g., pouches, foil pouches, etc.), each to store the sample fluid, aqueous fluid, air flush(s) or reagent, as shown, for example, in FIGS. 8, 13A, and 13B. In some embodiments, this design allows to achieve a linear actuation force and may offer cost benefits. Briefly, the sample preparation body comprises a cup serving as the sample chamber, and a cup comprising a plurality of bags (for example), each comprising at least one of aqueous fluid, reagent, or air. In some embodiments, the sample chamber comprises a piercing member. In some embodiments, the bags are sealed. In some embodiments, the bags are positioned on a chamber wall. Bags can have a burst point/stress concentrator 1302 that is positioned away from the sealing zones. A cap comprises a plunger, which comprises a channel to a filter and another chamber for the filtrate. As the user compresses (e.g., screws) the cap, the plunger first drives sample fluid through the plunger channel, and the sample chamber operates as a plunger having a piercing member, piercing each consecutive bag to selectively dispense its contents through the plunger channel.

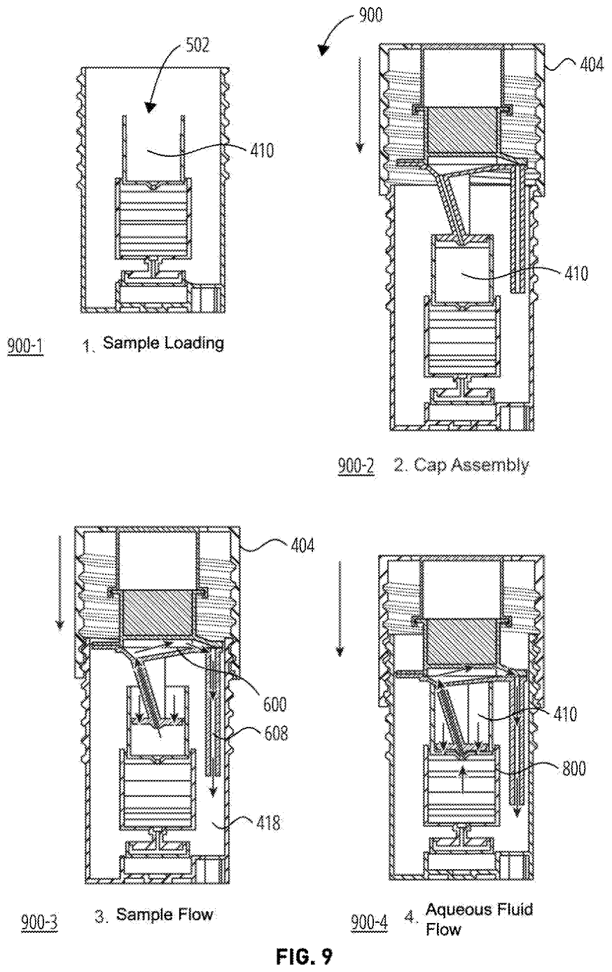

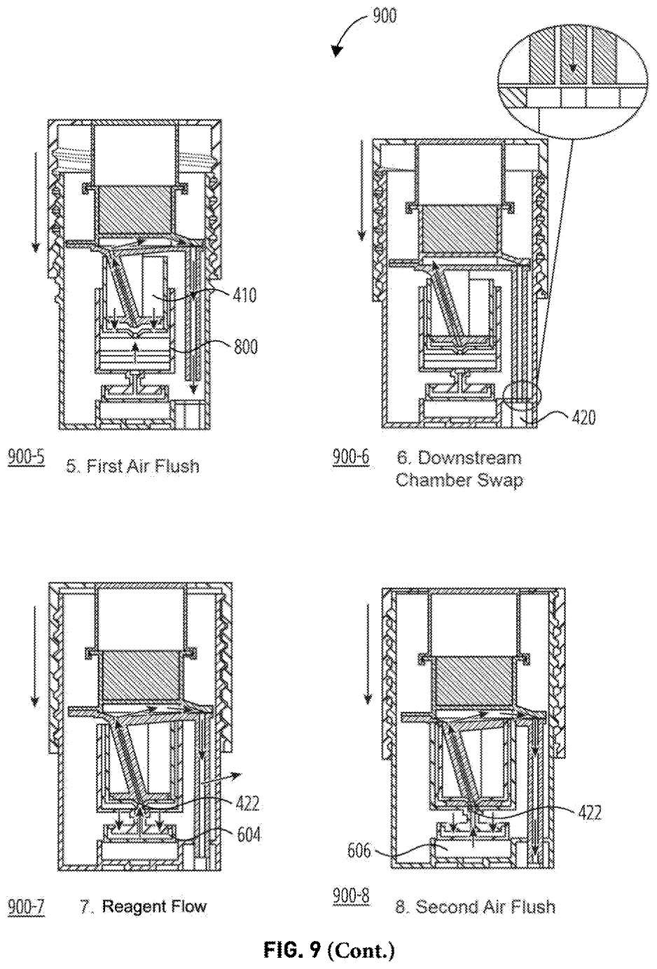

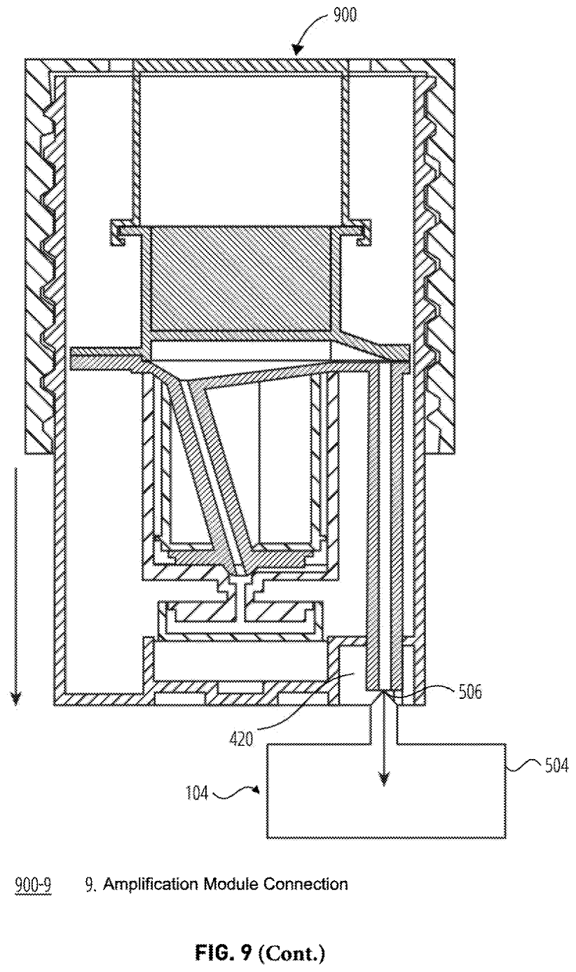

In an alternative embodiments, the bags, packs, or a deformable receptacle equivalent are stored on the inner walls of a chamber comprising a plunger, such that as the plunger moves into the chamber, the bags, packs, or a deformable receptacle are sequentially broken (e.g., punctured, pierced, torn, and equivalent means of releasing contents of the receptable) for selective dispensing. See, for example, FIG. 9.

Referring now to FIG. 9, a workflow using the sample preparation device described above in FIG. 8 is presented. At step 900-1, the sample chamber 410 may be filled with a sample 502. The sample may be about 5 mL in some embodiments. A user may manually fill the sample chamber with the sample.

At step 900-2, a user may screw the cap 404 of the sample preparation device 102 which may align the filter plunger 406 into the sample chamber.

At step 900-3, as the filter housing 416 travels down, the sample may be forced up through the filter 600, around an outlet spigot 608, and into the waste chamber 418.

At step 900-4, as the filter housing bottoms out, a piercing member may burst an aqueous fluid bag (e.g. 800). The sample chamber2 may act as a plunger to drive aqueous fluid up through the filter 600, around the outlet spigot 608, and into the waste chamber.

At step 900-5, as the sample chamber bottoms out, a piercing member may burst the first air bag (e.g. 800). The sample chamber may act as a plunger to drive air up through the filter, around the outlet spigot, and into the waste chamber.

At step 900-6, as an air flush is complete, the outlet spigot may pierce the breakable seal 422 such that all following fluid is directed into the prepared sample chamber 420.

At step 900-7, as the preceding chamber bottoms out, a breakable seal may burst. The second air chamber and/or the aqueous fluid chamber may act as a plunger to drive lysis up through the filter, around the outlet spigot, and into the prepared sample chamber 420.

At step 900-8, as the preceding chamber bottoms out, a breakable seal may burst. The reagent chamber may act as a plunger to drive air up through the filter, around the outlet spigot, and into the prepared sample chamber.

At step 900-9, a user may connect the sample preparation device 102 to an amplification module. An interface 504 may pierce an underside of the prepared sample chamber to allow flow of the reagent into an amplification module.

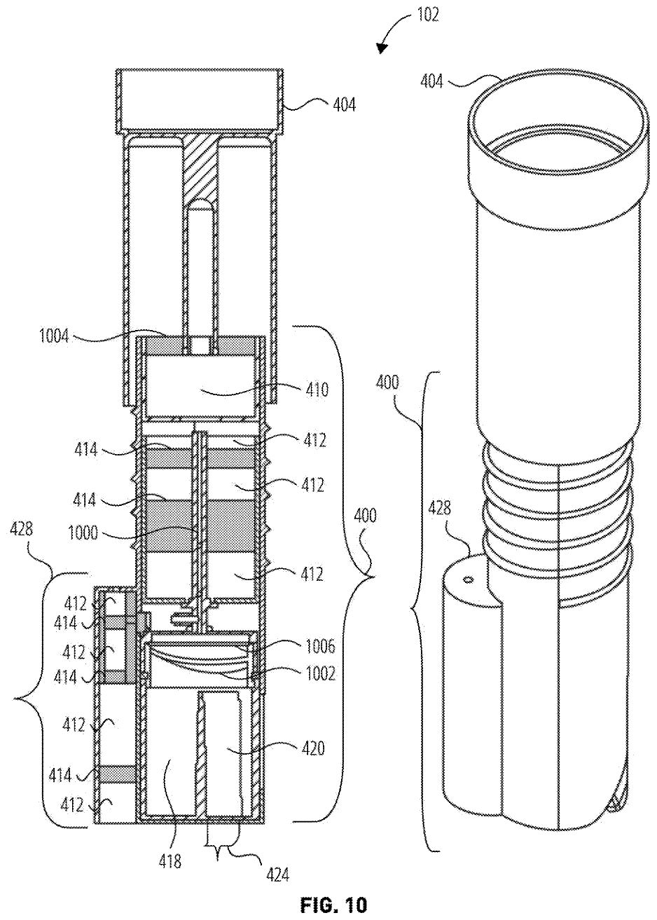

In some embodiments, a sample preparation system of the present disclosure utilizes a cap comprising a multi-part plunger 1004, a channel 1000 between the sample chamber 410, and a filter plenum 1002 that is breakable, as shown, for example, in FIG. 10. The sample fluid, aqueous fluid and first air flush are stored independently upstream of (e.g., above) the filter. A user compresses (e.g., screws) the cap, the multi-part plunger pushes the sample fluid through the channel into the multi-part filter 1006, and then further compression of the cap 404 moves a portion of the multi-part filter down to pressurize the two or more plungers forming aqueous fluid, reagent, and/or air chambers, thereby breaking the filter plenum and allowing e.g., a water and first air flush through the filter to the waste chamber.

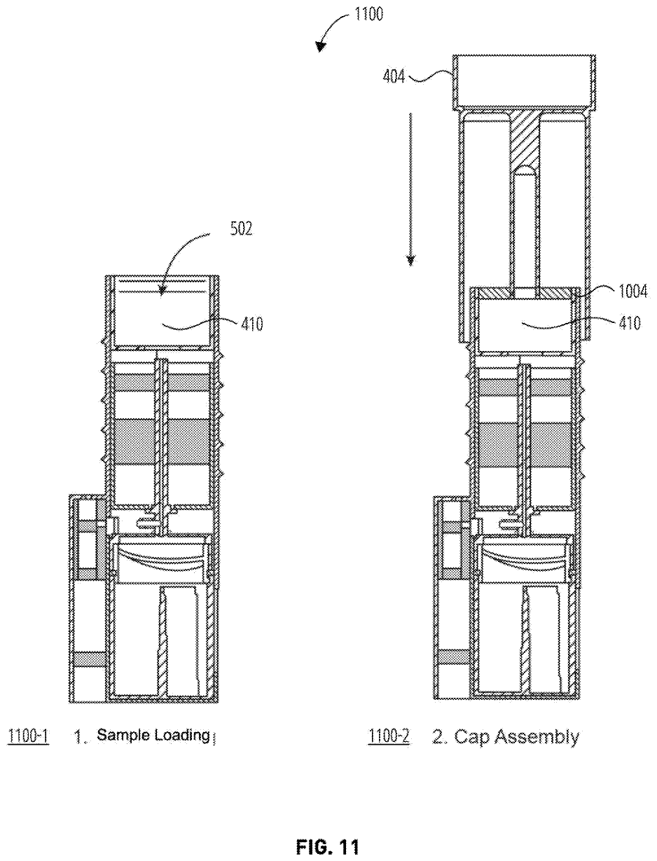

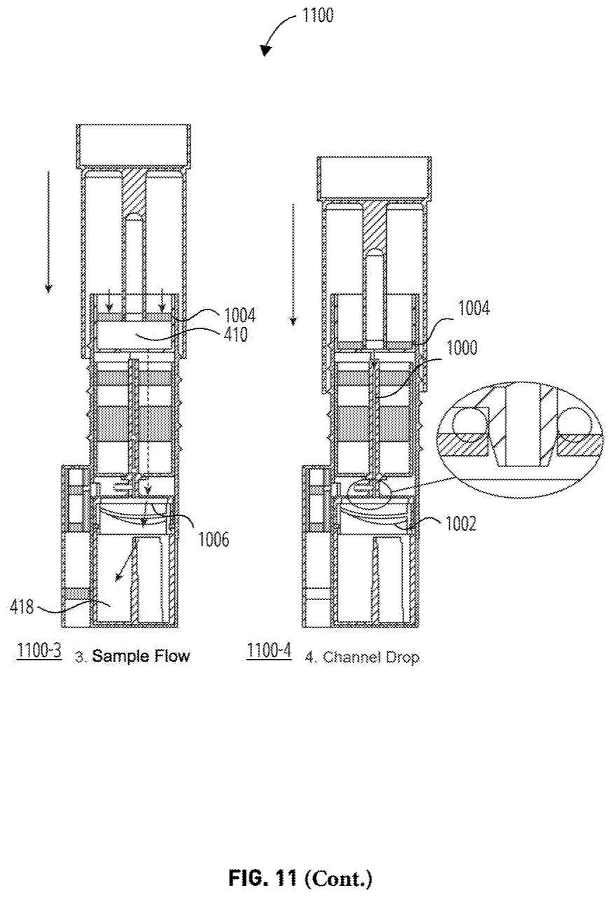

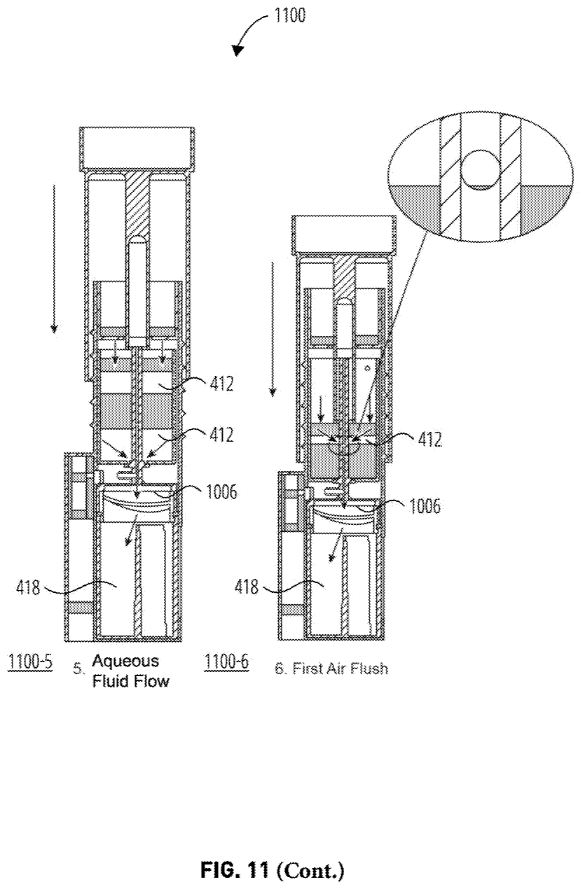

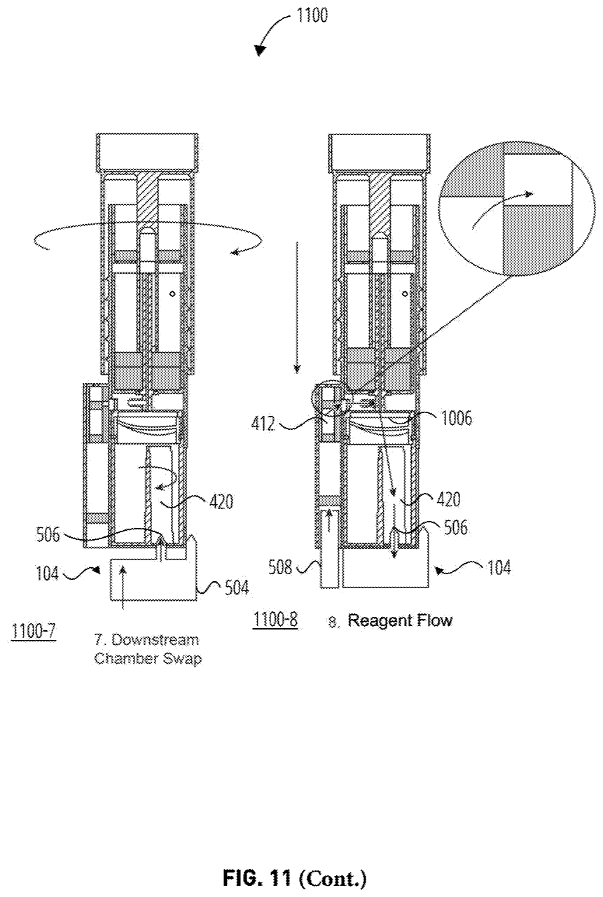

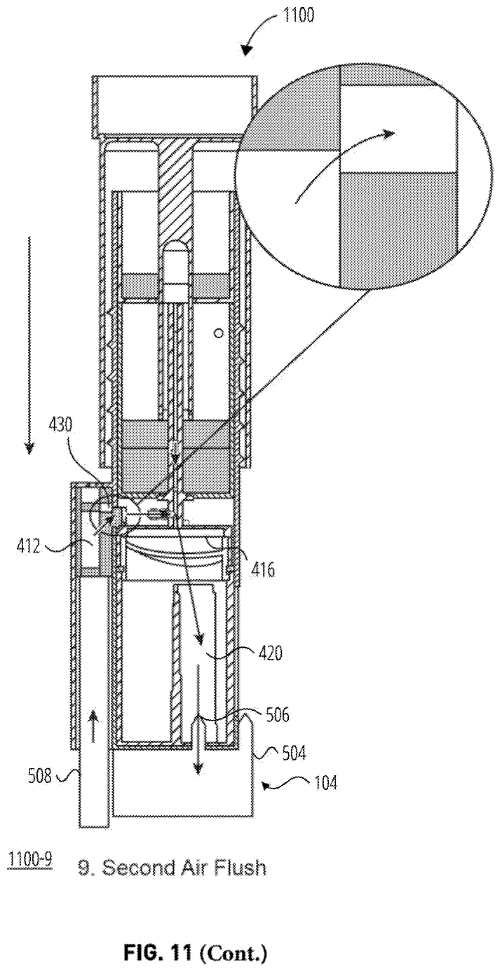

Reference is now made to FIG. 11, which illustrates a workflow using the sample preparation device 102 described above with reference to FIG. 10. At step 1100-1, a sample may be loaded into a sample chamber 410. A user may manually load a sample 502 into the sample chamber. A sample may be about 5 mL, in some embodiments.

At step 1100-2, a user may screw a cap 404 of the sample preparation device which may align a plunger into the sample chamber.

At step 1100-3, a multi-part plunger 1004 may travel downwards, which may force the sample through a channel 1000 into a filter housing comprising a filter, through the filter, and into a waste chamber 418.

At step 1100-4, as the multi-part plunger bottoms out in the sample chamber, one or more central portions of the multi-part plunger may continue to travel downwards, which may push the channel into the filter, bursting a breakable seal 422 in the process.

At step 1100-5, as the channel bottoms out, a central piece of the multi-part plunger may continued travelling downwards and may push on air and/or aqueous fluid plungers. Pressurization of an aqueous fluid chamber may break a seal on the channel, which may allow aqueous fluid to flow through the filter and into the waste chamber.

At step 1100-6, an air outlet may be exposed via a movement of the multi-part plunger. For instance, after the aqueous fluid has been pushed through, the multi-part plunger may expose an air outlet. Air from an air chamber may be passed into the channel, through the filter, and into the waste chamber.

At step 1100-7, a user may rotate the sample preparation device into an amplification module 104. A rotation of a base of the sample preparation device 102 may cause the reagent chamber 428 to become aligned with the filter outlet.

At step 1100-8, a user may push the sample preparation device onto the amplification module, which may push the reagent and air plungers upwards. As the top plunger moves, an outlet may be opened to pass the reagent through the filter, into the prepared sample chamber 420 for resuspension and into the amplification module.

At step 1100-9, as the sample preparation device continues to travel downwards on the reagent interface of the amplification cassette, an air chamber may be exposed to an outlet. An exposing of an air chamber to an outlet may allow for a flush of air through the filter, prepared sample chamber, and into the amplification module.

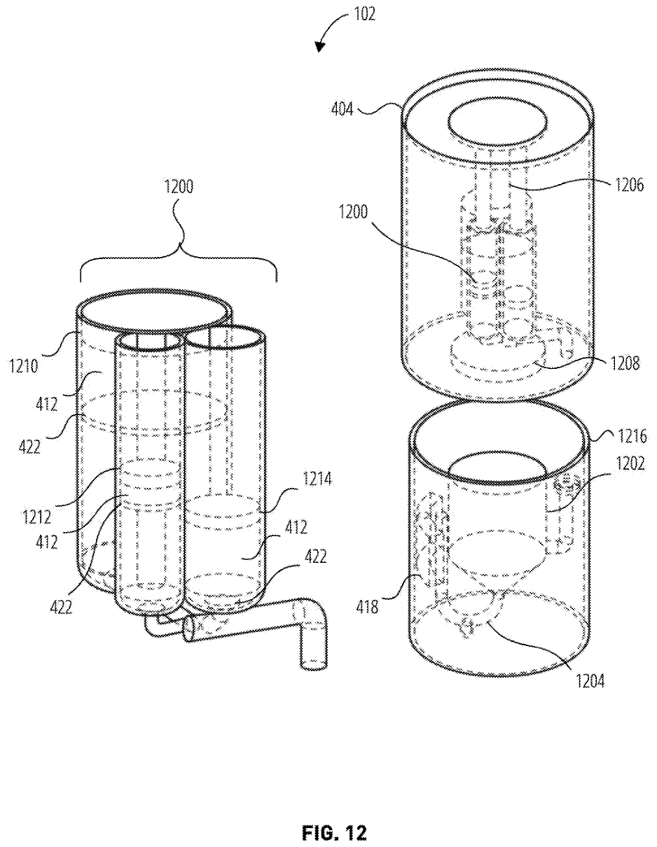

In some embodiments, a sample preparation device of the present disclosure utilizes a cap comprising a plurality of chambers, each comprising at least one of aqueous fluid, reagent, or air, and a sample preparation body comprising the sample chamber and a filter plunger, as shown, for example, in FIG. 12. The cap comprises a multi-plunger rod 1206 capable of projecting into each of the plurality of chambers, and the plurality of chambers each comprise a plunger 414, each of which have a position in the vertical plane that allows for sequential dispensing or selective dispensing.

In some embodiments, as exemplified in FIG. 12, a cap can comprise a multi-plunger rod 1206, a multi-chamber assembly 1200, a main plunger 1208. In some embodiments, within the multi-chamber assembly, there can be, e.g., a aqueous fluid plunger 1210, a lysate plunger 1212, a resuspension plunger 1214 each forming a chamber 412 with a breakable seal 422. Also shown in FIG. 12 is a base unit 1216, a collection chamber 1202, a u-bend 1204, and a waste chamber. One or more of any one of these features as shown in FIG. 12 are contemplated in a sample preparation device herein and can be combined with other embodiments described herein.

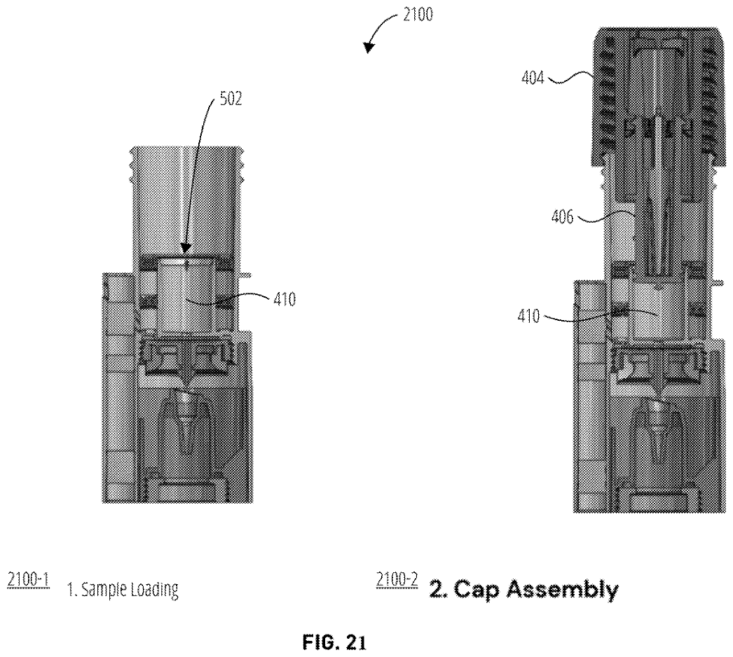

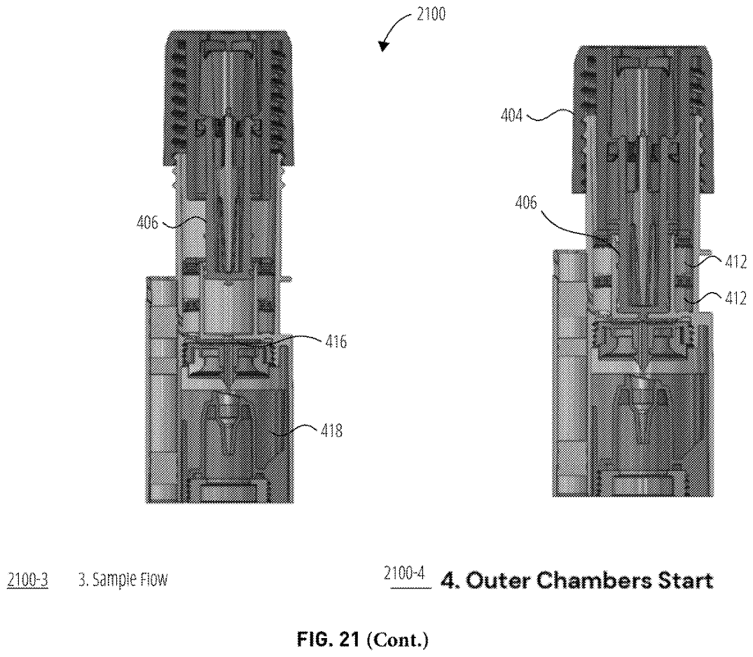

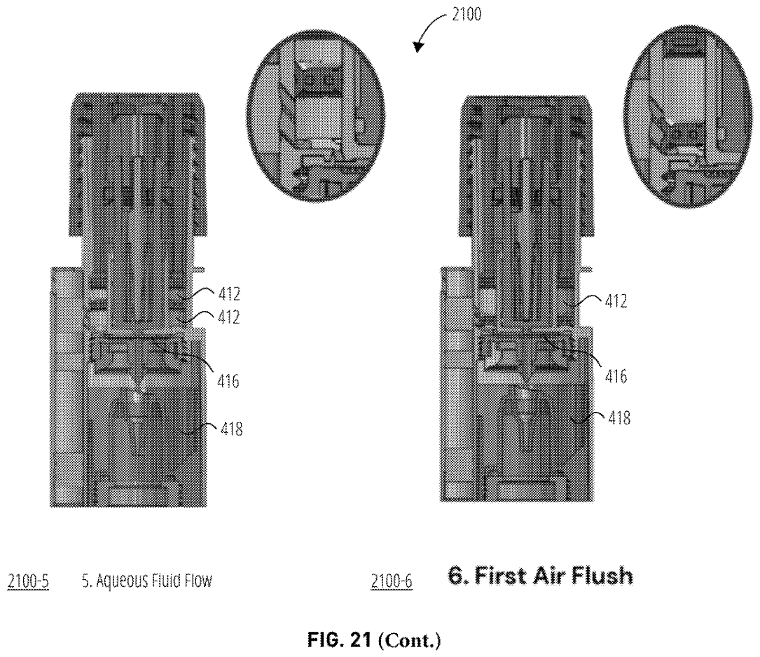

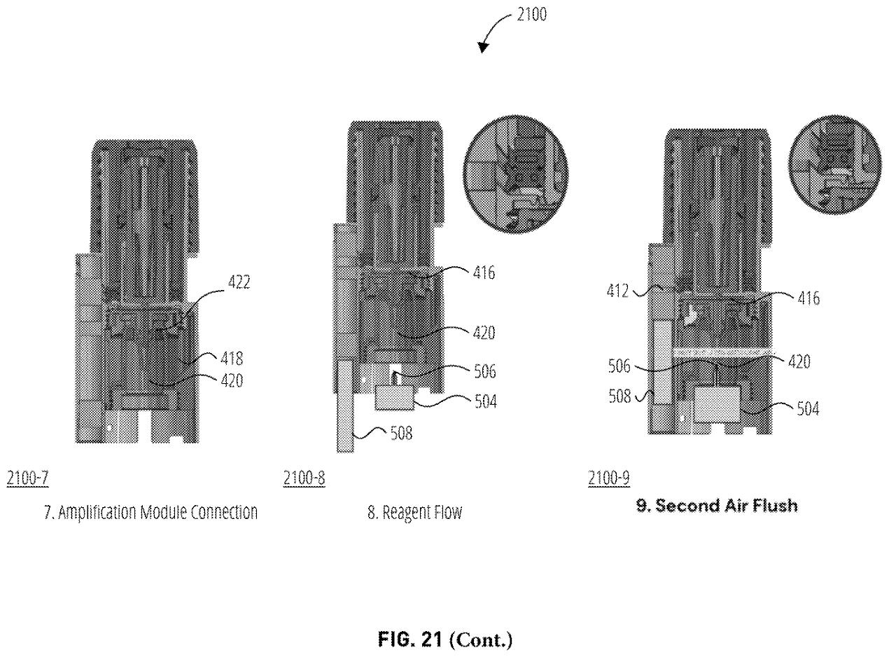

Referring now to FIG. 21, a workflow of another embodiment of a sample preparation device described herein is presented. At step 2104, a sample may be loaded into a sample chamber 410. About 5 mL or more of a sample may be loaded into the sample chamber. A sample 502 may be loaded into the sample chamber by a user.

At step 2106, a user may rotate the cap 404 of the sample preparation device 102. A rotation of the cap of the sample preparation device may cause the filter plunger 406 of the sample preparation device to become aligned with the sample chamber.

At step 2108, a flow of sample from the sample chamber may occur. For instance, the filter plunger may continue to travel towards the waste chamber 418, which may provide a pressure on the sample, causing the sample to travel through a filter 600 of a filter housing 416 of the sample preparation device. A portion or all of a sample may flow into the waste chamber 418.

At step 2112, one or more chambers 412 of the sample preparation device may become compressed. For instance, an air and/or aqueous fluid chamber may become compressed. Compression of one or more chambers may be cause by the cap slipping past a mechanical interface such that the cap may continue to travel downwards towards the waste chambers.

At step 2116, aqueous flow may occur. For instance, as the air chamber of the sample preparation device becomes compressed, pressure may act against the aqueous fluid chamber, which may cause a breakable seal 422 to open. An aqueous fluid may flow through a filter of the filter housing and into the waste chamber upon a breaking of the breakable seal.

At step 2118, an air flush may occur. For instance, once the aqueous fluid flows through the filter of the filter housing, a top of the plunger may move below a top of an inlet, which may allow air into the sample chamber. Air may be flushed through the filter of the filter housing.

At step 2122, an amplification module may be connected to the sample preparation device. A user may push downwards o the sample preparation device which may allow for a mating of the amplification device and the sample preparation device. A resuspension chamber of the sample preparation device may be driven upwards, which may pierce a breakable membrane and re-direct any fluid flow into the resuspension chamber.

At step 2124, an interface 504 of the amplification cassette may drive the reagent and air plungers upwards. As the top plunger moves, an outlet may become opened to pass lysis through the filter of the filter housing and into the prepared sample chamber 420 for resuspension and into the amplification module 104.

At step 2128, as the reagent chamber compresses the outlet to a second air chamber may become opened, which may further flush air through the filter of the filter housing.

In some embodiments, with reference to FIGS. 22A-22C, a sample preparation device 102 may comprise a cap 404, one or more plungers 414, one or more air chambers, a reagent chamber, a waste chamber, and/or a bottom plate. The one or more air chambers may comprise a first air chamber and a second air chamber. In some embodiments, an aqueous fluid chamber may be omitted from the sample preparation device and may operate with chambers consisting of one or more reagent chambers and one or more air chambers. In some embodiments, the bottom plate 2202 may be configured to move upwards towards the filter plunger 406. In some embodiments, an upwards movement of the bottom plate may actuate one or more plungers 414. In some embodiments, an upwards movement of the bottom plate may actuate a second air plunger, a reagent plunger, a first air plunger, and a filter plunger. In some embodiments, the reagent plunger may move upwards, which may cause the volume of reagent to move towards the sample chamber. A pressure of the reagent chamber may cause a one or more breakable seals 422 between the reagent chamber and the sample chamber and/or filter housing to burst, which may allow for a travelling of reagent into the filter housing 416 and/or sample chamber 410.

In some embodiments, the reagent chamber comprises an opening configured to be opened and closed (e.g., exposed and covered, exposed and occluded, etc.) upon movement of the plunger(s) in the reagent chamber. In one configuration, the opening fluidically connects the sample chamber and one of the plurality of chambers in the reagent chamber (not a waste chamber, e.g., an inner reagent chamber). In some embodiments, this is after the fluidic connection of the sample chamber and the amplification module. In such configuration, upon movement of the one or more plungers in the reagent chamber, the opening is exposed to selectively dispense contents of one of the plurality of chambers (e.g., reagent, e.g., lysate) into the sample chamber, through the filter, and into the amplification module.

### II.B. Diagnostic Mechanisms

The present disclosure also provides for sample preparation systems wherein the assembly has been separated into a single-use consumable sample preparation device and a separate reusable portion of the amplification module. This separation allows for simplified workflow and is more-cost effective since the reusable components of the testing device are sealed away from the liquid/assay and amplicon components of the test. The reusable portion of the amplification module has more complex and more costly components (e.g., heating elements, controller, etc.). The reusable portion of the amplification module can also be more challenging and more expensive to manufacture. It allows users to buy and replace the single-use consumable sample collection tube portion of the assembly in large numbers and/or at affordable cost while re-using the reusable portion of the amplification module, and in doing so, minimize risk of contamination between tests since the sample and amplicons remain sealed (e.g., hermetically) sealed away from the amplification module.

The amplification module as contemplated is easy to clean and reuse relative to pre-existing and disposable testing assay assemblies and devices.

Various diagnostic mechanisms are contemplated in these disclosed sample preparation systems. In some embodiments, the amplification module is used (and comprises reagents) for molecular diagnostics (e.g., reverse transcription polymerase chain reaction (RT-PCR), reverse transcriptase loop-mediated amplification (RT-LAMP), etc.). For example, in certain embodiments, the amplification module is utilized for molecular diagnostics, e.g., wherein a disease or an infection is identified by the nucleic acid of the pathogen (e.g., RNA, DNA) or biological markers (e.g., cell surface markers) associated with the infection or disease, mutations associated with the disease, etc. In certain embodiments, the amplification module is utilized (and comprises reagents) for immunologic diagnostics or antigenic diagnostics. In certain embodiments, the streamlined assay assemblies described herein utilize molecular diagnostic techniques (e.g., LAMP), thereby allowing for rapid pathogen or disease detection and results that emulate those obtained using the current gold standards for diagnosing the infections or diseases of interest.

In some embodiments, these devices can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process.

In some embodiments, the presence or absence of an analyte of interest (e.g., for a pathogen, cancerous cell, and/or precancerous cell) further comprises using a lateral flow assay (LFA). In some embodiments, the sample preparation system further comprises a readout module comprising, for example, an LFA strip. In some embodiments, the amplification module comprises an LFA strip.

### II.C. Breakable Barriers and Valves

As used, the term “breakable barrier” or “breakable seal” refers to a material seal that separates the contents of a first chamber from a second chamber. It is a seal that can be punctured, ruptured, broken, torn, pierced or penetrated once under pressure, e.g., when a piercing member is brought into direct contact with the seal under sufficient force or from air pressure as the cap is closed, thereby pressurizing the inside of the device. As used herein, the breakable barrier being “broken” refers to any one of these means for breaking the breakable barrier, e.g., piercing, tearing, penetrating, rupturing, etc. As one of skill in the art will appreciate, the breakable barrier can comprise one or more materials selected from the group including, but not limited to, foil (e.g., aluminum foil), plastics, foil-plastic laminates (e.g., plastic-backed foil), rubbers, membrane, polymer, paper, and cellophane. In certain embodiments, the breakable barrier comprises perforations marks (e.g., at the site at which the puncturing element is brought into direct contact with the seal) for puncturing, rupturing, breaking, tearing, piercing or penetrating of the breakable seal. In certain embodiments, the presences of the perforation marks on the allows for a clear puncture of the breakable seal with repeatable geometry and, in certain embodiments, without deformation of the puncturing element. In certain embodiments, this allows for bubble-free puncturing, as described herein.

As used, the terms “piercing member” and “puncturing element” can be used interchangeably and refer to a material that can rupture, pierce, puncture, tear or break the breakable seals described herein upon contact. In the present disclosure, where piercing or puncturing of the breakable seal are contemplated, other means to break the breakable seal are also contemplated (e.g., without limitation, rupturing, tearing, penetrating, slicing, cutting, ripping, etc.). Rods, needles, spears, spikes, and lances are non-limiting examples of puncturing elements contemplated in the subject disclosure. In some embodiments, the puncturing element is a hypotube. In some embodiments, the puncturing element can have a blunt tip, such that a surface area of the end of the second portion is orthogonal to the length of the puncturing element. In some embodiments, the puncturing element can have a tapered and/or sharp tip. In some embodiments, the puncturing element utilizes rotational movement to rupture, pierce, puncture, tear or break of the breakable barrier.

In some embodiments, a sample preparation device disclosed herein comprises a breakable barrier. In some embodiments, a breakable barrier is in any of the chambers contemplated herein. In some embodiments, that breakable barrier is on a prepared sample chamber. In some embodiments, the sample preparation device comprises a valve configured such that when a user actuates the valve, it operatively couples the second end of a sample chamber to the prepared sample chamber, thereby breaking the breakable barrier and fluidically connecting a second end of the sample chamber to the prepared sample chamber. In some embodiments, the breakable seal is broken by an outlet (e.g., a tapered outlet (e.g., a hollow piercing member)) on the sample chamber. In some embodiments, the breakable barrier is broken by a piercing member within the prepared sample chamber, whereby, for example, as the user compresses a cap, the piercing member is moved up towards the breakable seal. In some embodiments, the breakable seal is broken by a piercing member within an amplification module, whereby, for example, as the user compresses the cap, the piercing member is moved up towards the breakable barrier.

An interface can be made of suitable material, such as glass, ceramics, metals, paper, pressed cardboard, or polymers, but preferably comprises a plastic. In some embodiments, the interface and an outlet port are comprised of plastic material.

In some embodiments, the breakable barrier comprises foil. In some embodiments, the breakable barrier is adhesive backed, welded (e.g., with heat sealer or induction sealer), etc. In some embodiments, a breakable barrier can comprise a layered film with at least a metallic layer and a polymer, such as polypropylene-backed aluminum or polyethylene-backed aluminum. In some embodiments, a breakable barrier can comprise a three layer laminated material, such as e.g., polypropylene or polyethylene (in layer 1), aluminum (in layer 2), and polyester, PVC, or nylon (in layer 3).

In some embodiments, the breakable barrier has a thickness of about 50-200 μm. In some embodiments, the breakable barrier has a thickness of about 50-55 μm, about 55-60 μm, about 60-65 μm, about 65-70 μm, about 70-75 μm, about 75-80 μm, about 80-85 μm, about 85-90 μm, about 90-95 μm, about 95-100 μm, about 100-105 μm, about 105-110 μm, about 110-115 μm, about 115-120 μm, about 120-125 μm, about 125-130 μm, about 130-135 μm, about 135-140 μm, about 140-145 μm, about 145-150 μm, about 150-155 μm, about 155-160 μm, about 160-165 μm, about 165-170 μm, about 170-175 μm, about 175-180 μm, about 180-185 μm, about 185-190 μm, about 190-195 μm, about 195-200 μm, about 50-100 μm, about 50-125 μm, about 50-150 μm, or about 50-175 μm. In some embodiments, the breakable barrier has a thickness of about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm. In some embodiments, the breakable barrier has a thickness of about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm. In some embodiments, the breakable barrier has a thickness of about 50 μm, about 75 μm, about 100 μm, about 125 μm, about 150 μm, about 175 μm, or about 200 μm. In some embodiments, the breakable barrier has a thickness of about 50 μm, about 100 μm, about 150 μm, or about 200 μm.

In some embodiments, the interface, the outlet, and/or the outlet port are comprised of deformable material. In some embodiments either or both of the interface, the outlet and/or the outlet port are comprised of non-deformable material.

Using plastic components (e.g., in the interface) is a cost-effective manufacturing approach. However, plastics tend to be hydrophobic, which makes it difficult to ensure bubble-free filling. In some embodiments, the breakable barrier comprises a metallic material. In some embodiments, the piercing member comprises a plastic material and/or metallic material. In some embodiments, the piercing member is hydrophilic. The hydrophilicity of the piercing member counters the hydrophobicity of the plastic component and can aid in the reduction of bubbles.

The piercing member is made of a material having, at least, adequate hardness to puncture the breakable seal. For example, the piercing member can be made from a material having 2× the hardness of the breakable seal. In some embodiments, the hardness of the piercing member relative to the breakable seal is at least at least about 1×, about 2×, about 3×, about 4×, about 5×, about 6×, about 7×, about 8×, about 9×, about 10×, about 11×, about 12×, about 13×, about 14×, about 15×, or about 20× times the hardness of the material comprising the breakable seal, as measured using standard durometry methods. The discretion in hardness between the breakable barrier and the piercing member allows for a clear puncture of the piercing member with repeatable geometry. In certain embodiments, the piercing member provides for minimal deformation of the piercing member (or no deformation of the piercing member). Both of these minimize the possibility of bubble formation at the point the breakable barrier is broken. As used, “clear puncture” refers to an instantaneous puncturing, piercing, or breaking of the barrier of the entire surface of the piercing member in contact with the breakable barrier.

In some embodiments, a waste chamber provided herein can comprise an absorbent material, such as, but not limited to, vermiculite, absorbent pads, clay-based absorbents, oil-based absorbents, cotton, wool, paper, sponges, polyester, microfiber, superabsorbent polymers (SAPs), polyurethan foam, polypropylene fibers, silica gel, hydrogels, charcoal, ceramics, activate carbon, or any other type of absorbent. Waste can be isolated from a connection between the sample preparation device and the amplification module via the absorbent material. In some embodiments, an absorbent material positioned within the waste chamber can allow for a physical sequestering of waste within the waste chamber, which, e.g., minimizes or eliminates contamination of the lysed sample when the outlet on the filter housing (or sample chamber wherein it comprises a filter) to operatively connect to a breakable barrier or valve on the amplification module. In some embodiments, an absorbent material can take form of a circle, sphere, rectangle, hexagon, donut-shape, or other shapes. For instance, as a donut-shape the absorbent material can physically restrict waste from being positioned directly on top of the breakable barrier when the filter housing's outlet breaks the breakable barrier. In some embodiments, a breaking of the breakable barrier can allow a flow of aqueous fluid into the amplification module.

In some embodiments, a piercing member having a blunt tip, tapered tip, or sharp tip is contemplated. As used herein, the term “blunt tip” refers to a non-pointed or non-tapered end of the piercing member. Sharp tips and/or narrow blunt tips provide for a clear puncture in the breakable barrier with minimal deformation of the breakable barrier. Without being bound by mechanism or theory, the narrower the piercing member, the more effective it is at preventing bubble formation. In preferred embodiments, the surface area of an end of the second segment of the piercing member that is configured to contact the breakable barrier is less than 25% of the surface area of the breakable barrier, thereby limiting the size and/or number of gas bubbles formed during deformation of the breakable barrier and/or that can be introduced through the broken barrier. In some embodiments, the surface area of the end (tip) of the piercing member configured to contact the breakable barrier is less than 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% of the surface area of the breakable barrier.

In some embodiments, a valve is contemplated in place of the breakable barrier. In some embodiments, the prepared sample chamber comprises a wedged-ball valve, whereby in a first configuration, the wedged-ball valve seals off the interior of the prepared sample chamber, and in a second configuration, an element (e.g., the outlet of the sample chamber) compresses the wedged-ball valve, displacing it to unseal the interior of the prepared sample chamber. In some embodiments, the wedged-ball valve is comprised of glass. In some embodiments, the wedged-ball valve is comprised of rubber.

In some embodiments of the present disclosure, the valve e.g., is an actuable valve or a reversibly actuable valve. Such a valve may be incorporated in the device at the same location but instead of a breakable seal. As used herein, the term “valve” refers to a device component that allows for passage of fluids when it is actuated (on) and blocks the passage of fluid when it is not actuated (off). Non-limiting examples of valves include check valves, ball valves, butterfly valves, clapper valves, choke valves, diaphragm valves, solenoid valves, valve sequencers, multiway valves, and other suitable valves and similar devices.

In some embodiment, the breakable barrier is resealable.

### II.D. Filters

In some embodiments, the presently disclosed devices comprise a filter. In some embodiments, the filter has a pore size suitable for allowing fluid flow therethrough and for obstructing the flow of cells or suitable for obstructing the analyte prior to lysis. In the present disclosure, whenever a filter is contemplated, a matrix is contemplated as an alternative. In such embodiments, the matrix comprises a pore size suitable for allowing fluid flow therethrough and for obstructing the flow of cells or suitable for obstructing the analyte prior to lysis.

In some embodiments, the filter has a pore size no greater than 0.7 μm, 0.6 μm, 0.5 μm, 0.45 μm, 0.4 μm, 0.3 μm, 0.2 μm, or 0.22 μm. In some embodiments, the filter has a pore size no greater than 0.7 μm. In some embodiments, the filter has a pore size of about 0.65 μm. In some embodiments, the filter has a pore size of about 0.6 μm. In some embodiments, the filter has a pore size of about 0.55 μm. In some embodiments, the filter has a pore size of about 0.5 μm. The filter may have a pore size of at least 0.10 μm, preferably at least 0.20 μm.

The presently disclosed devices are designed such that the volume of the sample chamber is low (e.g., no greater than about 5 mL). In some embodiments, the volume of the sample chamber is no greater than about 3 mL, 4 mL, 5 mL, 6 mL, 7 mL, 8 mL, 9 mL, or 10 mL. In some embodiments, the volume of the sample chamber is no greater than about 3 mL, 4 mL, or 5 mL. In some embodiments, the volume of the sample chamber is no greater than 5 mL. In some embodiments, the volume of the sample chamber is about 3 mL, 4 mL, 5 mL, 6 mL, 7 mL, 8 mL, 9 mL, or 10 mL. In some embodiments, the volume of the sample chamber is about 3 mL, 4 mL, or 5 mL. It will be appreciated that low volumes (any that are no greater than 10 mL) may be used, as long as a sufficient sensitivity is obtained. These volumes allow for optimal pressure profiles. These volumes also allow for higher amplification (e.g., LAMP) efficiency. In some embodiments, the filter pore size and/or type of filter is selected based on the disease or condition being tested.

In preferred embodiments, the filter is selected based on having a low pressure and high amplification (e.g., LAMP) efficiency. In some embodiments, the filter is a Mixed Cellulose Ester (MCE) membrane filter, optionally 0.65 μm or 0.45 μm. In some embodiments, the filter is Polyethersulfone (PES) membrane filter, optionally 0.65 μm or 0.45 μm. In some embodiments, the filter is a glass microfiber filter (GF/F). In some embodiments, the filter is an MCE 0.65 μm filter. In some embodiments, a GF/F filter is used for Chlamydia trachomatis and Neisseria gonorrhoeae assays.

In some embodiments, the device comprises a pre-filter. For example, a whole urine, saliva, or mouthwash sample may be pre-filtered. Such pre-filtering may be performed in order to extract impurities (e.g., coarse impurities) from the sample before filtering the sample through the filter. In some embodiments, the pre-filter is selected to avoid filtering out bacteria. In some embodiments, the pore size of the pre-filter is at least at least about 10 μm, preferably at least about 20 μm. In some embodiments, the pre-filter preferably has a pore size no greater than about 250 μm, more preferably no greater than about 150 μm, even more preferably no greater than 80 μm.

In some embodiments, the sample preparation system is configured to allow for backwashing of the filter and/or pre-filter during filtration.

In some embodiments, the filter or matrix utilizes mechanical filtration. In some embodiments, the filter or matrix utilizes affinity-based filtration. In some embodiments, both mechanical and affinity-based filtration are contemplated. In some embodiments, the filter comprises an affinity capture reagent membrane. In some embodiments, the filter comprises an anion exchange membrane. In some embodiments, the filter comprises a cation exchange membrane. In some embodiments, the filter comprises a target-specific binding moiety. In some embodiments, the filter comprises protein A, protein G protein A/G, and/or protein L. In some embodiments, the filter is infused with a salt (e.g., buffering salt).

In some embodiments, the filter or matrix is capable of retaining analyte (prior to lysis) and free-floating nucleic acids. In some embodiments, the filter is capable of binding nucleic acids, e.g., a DNA-binding filter.

### II.E. Caps

The presently disclosed devices are contemplated having various types of caps. These caps can be operatively coupled to the sample preparation body using any one of (but not limited to) screwing-on (a screw cap), twisting-together (a twist-cap), snapping-on (a snap-cap), press-fitting, sliding together, or any combination thereof. For example, for a cap that engages with the sample preparation body by sliding thereon, one or both of the cap and the sample preparation body is contemplated, in some embodiments, having alignment features to guide the cap during engagement. For example, one or both of the cap and the sample preparation body can have reciprocating notches or ridges to prevent or minimize rotation and the user slides the cap onto the sample preparation body.

In some embodiments, the cap is a screw cap. In some embodiments, a screw cap is useful for allowing the user to generate adequate force to generate a sufficient delta pressure to filter the sample fluid. Without being bound by mechanism of action, in some embodiments, a significantly higher pressure is required to move the filter plunger through the sample chamber during filtration compared to moving the plurality of plungers forming one or more inner reagent chambers, aqueous fluid chambers, and/or air chambers.

In some embodiments, the cap comprises a first attachment element and the sample preparation body has a second attachment element. In some embodiments, the first attachment element comprises threading and the second attachment element comprises reciprocating threading configured to slidably receive the threading.

In some embodiments, the reciprocating threading is configured to slidably receive ½ to 10 complete rotations of the threading of the cap. In some embodiments, the sample collection tube is configured to slidably receive at least 1, 2, 3, 4, 5, 6, 7, 8, 9, or 10 complete rotations of the threading of the cap to complete filtration. In some embodiments, the sample collection tube is configured to slidably receive at least 3 to 10 complete rotations of the threading of the cap to complete filtration. In some embodiments, the sample collection tube is configured to slidably receive at least 3 to 6 or 3 to 5 complete rotations of the threading of the cap to complete filtration. In some embodiments, the sample collection tube is configured to slidably receive at least 3, 4, or 5 complete rotations of the threading of the cap to complete filtration. In some embodiments, the sample collection tube is configured to slidably receive at least 3, 4, 5, 6, 7, 8, 9, or 10 complete rotations of the threading of the cap to complete filtration. In some embodiments, the sample collection tube is configured to slidably receive between 2 to 10, 3 to 10, 4 to 10, 5 to 10, 6 to 10, 7 to 10, 8 to 10, 9 to 10, 2 to 9, 3 to 9, 4 to 9, 5 to 9, 6 to 9, 7 to 9, 8 to 9, 2 to 8, 3 to 8, 4 to 8, 5 to 8, 6 to 8, 7 to 8, 2 to 7, 3 to 7, 4 to 7, 5 to 7, 6 to 7, 2 to 6, 3 to 6, 4 to 6, 5 to 6, 2 to 5, 3 to 5, 4 to 5, 2 to 4, 3 to 4, or 2 to 3 complete rotations of the threading of the cap to complete filtration.

In some embodiments, the present disclosure also contemplates having ergonomic features on the cap. In some embodiments, the ergonomic feature is a handle on the cap, facilitating the engagement (e.g., pushing or screwing on). In some embodiments, the ergonomic feature comprises a knurling surface on at least a portion of the cap. In some embodiments, the ergonomic feature comprises one or more finger-grip indentations or finger-grip-enhancing features.

In some embodiments, the thread pitch is selected to control the maximum pressure that can be generated by a human hand rotation of approx. 90 deg.

In some embodiments, the thread pitch is varied to control the force needed to generate sufficient pressure to enable filtering. Filtering pressures can and do increase with use and therefor a reduced pitch of 10%, 30%, and even 50% can offset and maintain the user force needed to offset the increasing pressure over the filter life.

In some embodiments, the thread pitch is selected to be self-locking in order to limit reverse rotation if the user no longer applies or maintains a force on the cap.

In some embodiments, a capture feature is added to the thread to control maximum rotation or to trap the threads in a final position.

In some embodiments thread features are added to provide physical or audible feedback to the user to indicate certain stages of the process. In some embodiments, a user action is needed at these features such as a pause or hold.

In some embodiments the cap has axial linear features that allow the cap to move axially thru some or all of the cap motion.

### II.F. Reagents

In some embodiments, the chambers of the presently disclosed devices are contemplated having one or more reagents.

In some embodiments, a reagent comprises a washing buffer, a lysis buffer, a neutralization buffer, a resuspension buffer, an elution buffer, or a rehydration buffer.

In some embodiments, the reagent is a lysis buffer. In some embodiments, a lysis buffer comprises a lysing agent. In some embodiments, the lysis buffer comprises sodium hydroxide. Other, non-limiting examples of lysing agents include guanidinium thiocyanate, bioactive agents, such as lysing enzymes for lysing different cell types, such as lysozyme, leucopeptidase, lysostaphin, lip enzyme, kentalase (kitalase), cytolytic enzymes, and a variety of other lysing enzymes available from, for example, sigma-Aldrich corporation (St Louis, MO), as well as other commercially available lysing enzymes. Non-limiting examples of lysing agents include non-ionic detergents, anionic detergents, amphoteric detergents, low ionic strength aqueous solutions (hypotonic solutions), bacterial agents, aliphatic aldehydes, and antibodies that cause complement-dependent lysis.

In some embodiments, a lysis buffer comprises a salt. In some embodiments, the salt is a buffering salt (e.g., Tris-HCl) or an ionic salt (e.g., NaCl, KCl, or (NH₄)₂SO₄), which can be useful for regulating the pH and osmolarity of the lysate. Any one of these components could be in another reagent (other than the lysis buffer).

In some embodiments, the lysis buffer comprises a detergent (such as Triton X-100, SDS, Tween-20, etc.) to assist in breaking up membrane structures. Any one of these components could be in another reagent (other than the lysis buffer). In some embodiments, the lysis buffer is a detergent-free lysis buffer.

In some embodiments, the lysis buffer comprises an enzyme. These enzymes can enhance cellular digestion, to release the analyte. In some embodiments, the enzyme is a lysozyme, DNase, or RNase. In some embodiments, the enzyme is selected from the group consisting of PCR Biosystems IsoFast Bst DNA polymerase, PCR Biosystems IsoFast Warmstart Bst DNA polymerase, Empirical bioscience Bst DNA polymerase, LGC Bst DNA polymerase, New England Biolabs 2.0 Warmstart Bst DNA polymerase, New England Biolabs 3.0 Bst DNA polymerase, and New England Biolabs Full Length DNA polymerase. In some embodiments, the enzyme is NEB 3.0 Bst DNA polymerase or Empirical bioscience Bst DNA polymerase. In some embodiments, the enzyme is NEB 3.0 Bst DNA polymerase. Any one of these enzymes could be in another reagent (other than the lysis buffer).

In some embodiments, the enzyme is selected based on the disease or condition being tested. For example, in some embodiments, a presently disclosed device is used to test for Chlamydia trachomatis and/or Neisseria gonorrhoeae and the reagent used therein comprises NEB 3.0 Bst DNA polymerase or Empirical bioscience Bst DNA polymerase. In some embodiments, a presently disclosed device is used to test for Trichomonas vaginalis, and the reagent used therein comprises NEB 3.0 Bst DNA polymerase or Empirical bioscience Bst DNA polymerase.

In some embodiments, the lysis buffer further comprises metal ions, sugars (such as glucose), glycerol, metal chelators (e.g., EDTA), and/or reducing agents (e.g., dithiothreitol (DTT)). Any one of these components could be in another reagent (other than the lysis buffer).

In some embodiments, the lysis buffer comprises about 250 mM of NaOH. In some embodiments, the lysis buffer comprises about 10 mM to 250 mM of NaOH. In some embodiments, the lysis buffer comprises about 10 mM to 300 mM of NaOH. In some embodiments, the lysis buffer comprises about 50 mM to 250 mM of NaOH. In some embodiments, the lysis buffer comprises about 50 mM to 300 mM of NaOH. In some embodiments, the lysis buffer comprises about 10 mM to 300 mM, 20 mM to 300 mM, 30 mM to 300 mM, 40 mM to 300 mM, 50 mM to 300 mM, 60 mM to 300 mM, 70 mM to 300 mM, 80 mM to 300 mM, 90 mM to 300 mM, 100 mM to 300 mM, 110 mM to 300 mM, 120 mM to 300 mM, 130 mM to 300 mM, 140 mM to 300 mM, 150 mM to 300 mM, 160 mM to 300 mM, 170 mM to 300 mM, 180 mM to 300 mM, 190 mM to 300 mM, 200 mM to 300 mM, 210 mM to 300 mM, 220 mM to 300 mM, 230 mM to 300 mM, 240 mM to 300 mM, 250 mM to 300 mM, 260 mM to 300 mM, 270 mM to 300 mM, 280 mM to 300 mM, or 290 mM to 300 mM of NaOH. In some embodiments, the lysis buffer comprises about 10 mM to 300 mM, 30 mM to 300 mM, 50 mM to 300 mM, 70 mM to 300 mM, 90 mM to 300 mM, 110 mM to 300 mM, 130 mM to 300 mM, 150 mM to 300 mM, 170 mM to 300 mM, 190 mM to 300 mM, 210 mM to 300 mM, 230 mM to 300 mM, 250 mM to 300 mM, 270 mM to 300 mM, or 290 mM to 300 mM of NaOH. In some embodiments, the lysis buffer comprises about 10 mM to 250 mM, 20 mM to 250 mM, 30 mM to 250 mM, 40 mM to 250 mM, 50 mM to 250 mM, 60 mM to 250 mM, 70 mM to 250 mM, 80 mM to 250 mM, 90 mM to 250 mM, 100 mM to 250 mM, 110 mM to 250 mM, 120 mM to 250 mM, 130 mM to 250 mM, 140 mM to 250 mM, 150 mM to 250 mM, 160 mM to 250 mM, 170 mM to 250 mM, 180 mM to 250 mM, 190 mM to 250 mM, 200 mM to 250 mM, 210 mM to 250 mM, 220 mM to 250 mM, 230 mM to 250 mM, or 240 mM to 250 mM of NaOH. In some embodiments, the lysis buffer comprises about 10 mM to 250 mM, 30 mM to 250 mM, 50 mM to 250 mM, 70 mM to 250 mM, 90 mM to 250 mM, 110 mM to 250 mM, 130 mM to 250 mM, 150 mM to 250 mM, 170 mM to 250 mM, 190 mM to 250 mM, 210 mM to 250 mM, or 230 mM to 250 mM of NaOH. In some embodiments, the lysis buffer comprises about 10 mM, 30 mM, 50 mM, 70 mM, 90 mM, 110 mM, 130 mM, 150 mM, 170 mM, 190 mM, 210 mM, 230 mM, 250 mM, or 270 mM of NaOH. In some embodiments, the lysis buffer comprises about 10 mM, 40 mM, 70 mM, 100 mM, 130 mM, 160 mM, 190 mM, 220 mM, or 250 mM of NaOH.

In some embodiments, the lysis buffer comprises about 50 mM, 80 mM, 110 mM, 140 mM, 170 mM, 200 mM, 230 mM, or 260 mM of NaOH. In some embodiments, the lysis buffer comprises about 50 mM, 100 mM, 150 mM, 200 mM, or 250 mM of NaOH.

In some embodiments, the reagent is a resuspension buffer. In some embodiments, the resuspension buffer comprises one or more of Tris-HCl, (NH₄)₂SO₄, Tween-20, and MgSO₄. In some embodiments, the resuspension buffer comprises one or more of Tris-HCl, pH 8.0, EDTA, and an enzyme (e.g., RNase, e.g., RNase A). In some embodiments, the resuspension buffer comprises Tris-HCl. In some embodiments, the resuspension buffer comprises a high Tris-HCl, such that it has adequate capacity to reduce the pH of the final reaction mix. For example, in some embodiments, the concentration of Tris-HCl is about 50 mM. In some embodiments, the concentration of Tris-HCl is about 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, and 150 mM. In some embodiments, the concentration of Tris-HCl is about 50 mM, 60 mM, 70 mM, 80 mM, 90 mM, 100 mM, 110 mM, 120 mM, 130 mM, 140 mM, 150 mM. In some embodiments, the volumetric ratio of lysis buffer: resuspension buffer is about 1:1. In some embodiments, the volumetric ratio of lysis buffer: resuspension buffer is about 1:1, 1:2, 1:3, 1:4, or 1:5. In some embodiments, the volumetric ratio of lysis buffer: resuspension buffer ranges from about 1:1 to 1:4. In some embodiments, the volumetric ratio of lysis buffer: resuspension buffer is about 2:1.

In some embodiments, the resuspension buffer comprises approximately the following.

| | |
|---|---|
| Tris-HCl | 50 mM |
| (NH₄)₂SO₄ | 30 mM |
| Tween-20 | 0.1% |
| MgSO₄ | 6 mM |

In some embodiments, the lysis buffer comprises about 250 mM of NaOH.

In some embodiments, the resuspension buffer comprises approximately the following:

| | |
|---|---|
| Tris-HCl | 62.5 mM |
| (NH₄)₂SO₄ | 37.5 mM |
| Tween-20 | 0.125% |
| MgSO₄ | 7.5 mM |

In some embodiments, the preceding composition is used for a 1:4 lysis buffer to resuspension buffer volumetric ratio. In some embodiments, the lysis buffer comprises about 250 mM of NaOH.

In some embodiments, the resuspension buffer comprises approximately the following:

| | |
|---|---|
| Tris-HCl | 100 mM |
| (NH₄)₂SO₄ | 60 mM |
| Tween-20 | 0.2% |
| MgSO₄ | 12 mM |

In some embodiments, the preceding composition is used for a 1:4 lysis buffer to resuspension buffer volumetric ratio. In some embodiments, the lysis buffer comprises about 250 mM of NaOH.

In some embodiments, the reagent is a washing buffer comprises EtOH. The washing buffer helps remove chaotropic agents.

In some embodiments, the regarding is a rehydration buffer. Rehydration buffers can comprise, for example, one or more selection from the group consisting of ddH₂O, TE buffer, and Tris-HCl, pH 8.5.

Typically, following lysis there is a need to a neutralization buffer, since the pH of the lysate is very high (alkaline). The present disclosure allows for the use of resuspension buffers and lysis buffer: resuspension buffer volumetric ratios that eliminate the need for a neutralization buffer. In such embodiments, the resuspension buffer reduces the pH of the lysate, thereby elimination or reducing the need for a neutralization buffer. This further contributes to the ease of manufacturing and ease of use of the device, since lower reagent volumes are required in the device, less force generation is required from the user, and fewer inner reagent chambers are needed in the device design. It further contributes to the portability of the device.

In some embodiments, the sample preparation device does not have neutralization buffer. In some embodiments, the amplification module comprises neutralization buffer or a neutralization component, optionally in lyophilized form. In further embodiments, a sample preparation system can transmit lysate directly from the filter and sample chamber to the amplification module-without the need for a prepared sample chamber.

### II.G. Applications and Streamlined Preparation to Results

The devices and systems described herein are useful as portable, test-to-result, disposable, inexpensive devices that have applications across a broad range of disciplines and sectors. The simplified disposable device components keep manufacturing and product costs low. This device has simpler manufacturing design and also cost effective. The simplified design eliminates the need for complex features, such as manually and individually actuated complex valve systems (as opposed to the automated progressive use of a valve-like feature), multiple microfluidic channels, etc. The simplified design still allows to isolate and then selectively dispense various fluids (e.g. reagent, aqueous fluid, air, etc.) (optionally dissimilar volumes), with built-in temporal control of when each is dispensed. The simplified design also allows storage of multiple fluids within the same module, e.g. in close proximity and/or adjacent to one another while still allowing for precise dispensing at low sample volumes and/or minimizing cross-contamination between fluids and/or minimizing pressure required from user to actuate the device. The single use of the disclosed devices obviates the need for cleaning the device after use and minimizes the risk of contaminating new samples, as seen in reusable devices. Following use, the device can be disposed of using protocol established to avoid the spread of infection.

The presently disclosed devices are also designed to provide significant improvement in sample analyte detection (e.g., sensitivity) and ease of use. Regarding ease of use, the device is designed to be used by a lay person without the need for a clinician or laboratory specialist. For example, the dual-purpose plungers, as disclosed herein, enable easier operation of a multi-reagent, multicomponent, time-sensitive, multi-step assay device by an lay person, increasing that they will perform the test accurately on the first try. The devices have high usability, as determined by usability factors. Non-limiting examples of usability factors include ease of learning (the speed with which a user, new to the device, can operate and perform the test correctly), efficiency of use (the speed with which a user can operate device from sample collection to result), error frequency and severity (how often users make errors in operating the device and the severity of those errors), user satisfaction, etc. Other usability factors for which the scores well include ergonomics, shelf-life, and affordability. The devices and systems described herein also eliminate the chance of sample degradation, as the sample is introduced to the system just after capture and does not need to be preserved or transported like lab specimens. Furthermore, they provide better control of conditions, we reduce variable conditions that occur with sample shipping or transportation.

The presently disclosed devices are suited for rapid deployment of testing equipment, decentralized testing, telemedicine, and wide-spread accessibility. Unlike conventional testing devices, the assemblies disclosed herein were designed to not require a skilled clinician/technician, making them ideal at-home testing devices for lay users.

In some embodiments, the present disclosure contemplates that the sample preparation systems are for single-use testing. Biological samples are collected by the user and testing can be performed outside of a laboratory setting (e.g., at home). In some embodiments, the stream-lined actuation of the disclosed sample preparation systems allow for reduced set up and run time. The systems allow the user to bypass conventional sample transportation and purification steps. In some embodiments, the user can run the test for a shortened length of time (e.g., less than 30 minutes). The user also gets an immediate test result. This streamlined assay design allows for rapid assay set up (e.g., less than 1 minute).

In some embodiments, the total preparation and testing time is up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 minutes. In some embodiments, the total testing time is less than 10, 15, 20, 25, or 30 minutes. In some embodiments, the total testing time is up to 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes. As used, testing time can refer to time from insertion of the sample fluid into the sample chamber to first result display (e.g., LFA readout).

### III. Diseases and Conditions

Non-limiting examples of sexually transmitted diseases include bacterial vaginosis, chancroid (Haemophilus ducreyi), chlamydia (Chlamydia trachomatis), gonorrhea (Neisseria gonorrhoeae), Granuloma inguinale, Lymphogranuloma venereum, syphilis (Treponema pallidum), trichomoniasis (Trichomonas vaginalis), genital herpes, genital warts, crabs (Pthirus pubis), scabies, and molluscum contagiosum virus (MCV). Non-limiting examples of sexually transmitted diseases caused by viral agents include genital herpes (caused by herpes simplex viruses, or HSVs: herpes simplex viruses type 1 (HSV-1) and type 2 (HSV-2)); herpes labialis (cold sores); AIDS (caused by human immunodeficiency virus, or HIV); genital warts (caused by human papillomaviruses, or HPVs), spastic paralysis and adult T cell leukemia (caused by human T-cell leukemia or lymphotropic virus type 1 (HTLV-1)), and viral hepatitis (caused by hepatitis viruses, such as hepatitis B virus (HBV) and hepatitis C virus (HCV).

In one embodiments, the disease or condition refers to a microbial infection of the urinary tract. As used herein, a “urinary tract infection” refers to an infection of the urinary system, kidneys, bladder, or urethra.

The devices and methods are also useful for detection of cancer or precancerous conditions.

In some embodiments, the cancer is a solid tumor. In some embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastoma, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

### IV. Urine Collection Methods

A recurring challenge with the pre-existing methods for urine sampling is low sensitivity to counterpart collection methods, e.g., swabs. This is particularly problematic in the context of disease/condition tests that require a dirty catch sample, e.g., tests for sexually transmitted diseases. Urine-based sexually transmitted disease (STD) tests confer the best sensitivity when urine is captured from the urethra-thereby capturing pathogens that tend to cumulate in the urethra, as opposed to the bladder. The capture of the initial flow of urine is known in the art as a “dirty catch” or “first catch.” Generally, when conducting urine tests, patients are instructed to micturate into a cup (typically of large volume, e.g., about 100 mL or more). In some cases, standard urine sample cups used in the field range from 3.5-6.5 ounces. When testing for STDs, the patients are specifically instructed to capture the initial flow of urine. When testing for urinary tract infections, patients are instructed to capture urine that is midstream and are often instructed to disinfect (e.g., with a wipe) their genitals before micturating to avoid contaminating the sample. When testing for precancerous or cancerous cells, patients are can provide any of their urine flow (dirty catch, midstream, or otherwise). In all these cases, the user micturates freely (similar to how they would regularly void) into a specimen cup and the urine is often diluted prior to filtration and testing. A comparatively small volume of the urine collected from the patient is tested, in part because of the difficulty of filtering large volumes and increased likelihood of filter clogging with large volumes. Given the standard practice of collecting a large volume of urine from patients and then having a skilled operator extract a small sample testing volume therefrom, urine tests tend to have low sensitivity. The practice results in a frequently inadequate concentration of analytes than one would get if the patient was able to only provide an initially very small and concentrated urine sample volume (e.g., a small volume dirty catch).

The inventors of the present disclosure have developed a method for collecting a small volume urine sample for diagnostic testing, wherein the user is given a small volume cup (e.g., significantly smaller than a standard urine sampling cup) and instructed to micturate into the cup by starting and then stopping their urine flow instead of freely voiding the usual large sample volume collected for standard urinalysis (e.g., 100 mL or more). While this capture is counter-intuitive to current standard urine collection methods, it allows the user to capture a very small, concentrated (useful with respect to e.g., STD pathogens) urine sample, likely to yield higher analyte detection sensitivity. In some embodiments, the cup is smaller than a standard urine sample cup. In some embodiments, the cup provided to the user is less than about 100 mL. In some embodiments, the cup provided to the user is less than about 95 mL. In some embodiments, the cup provided to the user is less than about 90 mL. In some embodiments, the cup provided to the user is less than about 80 mL. In some embodiments, the cup provided to the user is less than about 70 mL. In some embodiments, the cup provided to the user is less than about 65 mL. In some embodiments, the cup provided to the user is less than about 60 mL. In some embodiments, the cup provided to the user is less than about 50 mL. In some embodiments, the cup provided to the user is less than about 40 mL. In some embodiments, the cup provided to the user is less than about 30 mL. In some embodiments, the cup provided to the user is less than about 20 mL. In some embodiments, the cup provided to the user is less than about 10 mL. In some embodiments, the cup provided to the user is about 10 mL to 90 mL. In some embodiments, the cup provided to the user is about 10 mL to 20 mL, 10 mL to 25 mL, 10 mL to 30 mL, 10 mL to 35 mL, 10 mL to 40 mL, 10 mL to 45 mL, 10 mL to 50 mL, 10 mL to 55 mL, 10 mL to 60 mL, 10 mL to 65 mL, 10 mL to 70 mL, 10 mL to 75 mL, 10 mL to 80 mL, 10 mL to 85 mL, or 10 mL to 90 mL. In some embodiments, the cup provided to the user is about 5 mL to 10 mL, 20 mL, 5 mL to 25 mL, 5 mL to 30 mL, 5 mL to 35 mL, 5 mL to 40 mL, 5 mL to 45 mL, 5 mL to 50 mL, 5 mL to 55 mL, 5 mL to 60 mL, 5 mL to 65 mL, 5 mL to 70 mL, 5 mL to 75 mL, 5 mL to 80 mL, 5 mL to 85 mL, or 5 mL to 90 mL. In some embodiments, the cup provided to the user is less than about 5 mL to 10 mL, 5 mL to 15 mL, 10 mL to 15 mL, 5 mL to 20 mL, 10 mL to 20 mL, 20 mL to 30 mL, 15 mL to 20 mL, 15 mL to 30 mL, or 20 mL to 30 mL. In some embodiments, the cup provided to the user is less than about 5 mL to 30 mL, 5 mL to 40 mL, 5 mL to 50 mL, or 5 mL to 60 mL. In some embodiments, the cup provided to the user is less than about 10 mL to 30 mL, 10 mL to 40 mL, 10 mL to 50 mL, or 10 mL to 60 mL.

In some embodiments, the cup provided to the user comprises one or more indicators (e.g., a surface marking (e.g., line, arrow, dot, a 2D-shape, etc.)). In some embodiments, the indicator is visible on the surface of the cup (e.g., inner surface and/or outer surface). For example, a cup could comprise two lines, whereby the user is instructed to stop urine flow once the collected urine volume is between the two lines. In some embodiments, the use of two lines or an indicator illustrating a range within which the user can micturate (referred to as the “indicated minimum and maximum volume”) provides better results than a single line or indicator for a single point in the cup. Without being bound by mechanism, in some embodiments, two lines or an indicator illustrating a range within which to micturate, increases the user's confidence in their ability to correctly capture the sample and increases precision of the collection method. In some embodiments, the indicated minimum and maximum volume is about 5 mL-20 mL, 5 mL-15 mL, or 5 mL-10 mL. In some embodiments, the indicated minimum and maximum volume is about 6 mL-20 mL, 6 mL-15 mL, or 6 mL-10 mL. In some embodiments, the indicated minimum and maximum volume is about 7 mL-20 mL, 7 mL-15 mL, or 7 mL-10 mL. In some embodiments, the indicated minimum and maximum volume is about 8 mL-20 mL or 8 mL-15 mL. In some embodiments, the indicated minimum and maximum volume is about 8 mL-20 mL, 9 mL-20 mL, 10 mL-20 mL, 11 mL-20 mL, 12 mL-20 mL, 13 mL-20 mL, 14 mL-20 mL, 15 mL-20 mL, or 16 mL-20 mL. In some embodiments, the indicated minimum and maximum volume is about 8 mL-25 mL, 9 mL-25 mL, 10 mL-25 mL, 11 mL-25 mL, 12 mL-25 mL, 13 mL-25 mL, 14 mL-25 mL, 15 mL-25 mL, or 16 mL-25 mL. In some embodiments, the indicated minimum and maximum volume is about 7 mL-21 mL, 8 mL-21 mL, 9 mL-21 mL, 10 mL-21 mL, 11 mL-21 mL, 12 mL-21 mL, 13 mL-21 mL, 14 mL-21 mL, 15 mL-21 mL or 16 mL-21 mL. In some embodiments, the indicated minimum and maximum volume is about 10 mL-20 mL. In some embodiments, the indicated minimum and maximum volume is about 11 mL-20 mL. In some embodiments, the indicated minimum and maximum volume is about 12 mL-20 mL. In some embodiments, the indicated minimum and maximum volume is about 13 mL-20 mL. In some embodiments, the indicated minimum and maximum volume is about 14 mL-20 mL. In some embodiments, the indicated minimum and maximum volume is about 15 mL-20 mL. In some embodiments, the indicated minimum and maximum volume is about 15 mL-25 mL. In some embodiments, the indicated minimum and maximum volume is about 15 mL-30 mL.

In some embodiments, the maximum volume collected in the cup is no more than about 10 mL. In some embodiments, the maximum volume collected in the cup is no more than about 15 mL. In some embodiments, the maximum volume collected in the cup is no more than about 20 mL. In some embodiments, the maximum volume collected in the cup is no more than about 21 mL. In some embodiments, the maximum volume collected in the cup is no more than about 22 mL. In some embodiments, the maximum volume collected in the cup is no more than about 23 mL.

In some embodiments, any volume cup can be used for the urine collector so long as the indicated minimum and maximum volume is as disclosed herein.

In some embodiments, the user is then instructed to pour all or a portion of the urine into the sample collector for a presently disclosed sample preparation system. For example, the user collects urine in a 60 mL cup and is instructed to pour a portion of it (e.g., 5 mL, as indicated by a fill line within the sample chamber) into the sample chamber. In some embodiments, the user is instructed to fill the sample chamber. In some embodiments, the sample preparation device has an overflow protection systems as described herein.

In some embodiments, the present disclosure provides a method comprising one or more of the following steps:
(i) providing a sample preparation device comprising a filter,
(ii) providing a urine sample that was collected by having the user micturate into a sample collector by initiating and then stopping their flow of urine into the sample collector, wherein the sample collector has a volume of no more than about 70 mL, optionally about 20 mL, 30 mL, 40 mL, 50 mL or 60 mL; alternatively, providing a urine sample that was collected by having the user micturate into a sample collector comprising indicators for an indicated minimum and maximum volume, wherein the user micturates by initiating and then stopping their flow of urine into the sample collector within the indicated minimum and maximum volume;
(iii) pouring all or a portion of (e.g., a predetermined volume from) the urine sample into the sample preparation device (e.g., the sample chamber) and filtering the contents thereof, thereby retaining an analyte on the filter, optionally wherein the predetermined volume is no more than about 5 mL;
(iv) applying a lysing agent to the filter, thereby producing a lysate comprising nucleic acid molecules;
(v) amplifying nucleic acid molecules, and based on said amplifying;
(vi) determining the presence or absence of a pathogen, cancerous cell, and/or precancerous cell.

The devices and methods disclosed herein provide higher sensitivity for urine test results compared to urine collected by standard practice. Additionally, the devices and methods disclosed herein can process more sample (e.g., urine) than standard tests, e.g., in some embodiments, 5 mL samples are processed herein versus the standard 0.5-0.1 mL processed in standard tests. The methods disclosed also have the advantage of acceptable usability and more reliable performance/accuracy for the users.

### V. Kits

The embodiments disclosed herein also include kits including the subject devices and which can be used according to the subject methods. The subject kits can include two or more, e.g., a plurality, three or less, four or less, five or less, ten or less, or fifteen or less, or fifteen or more, of the devices or device components assay assembly components disclosed herein, according to any of the embodiments described herein, or any combinations thereof.

The kits can include one or more compositions and/or reagents, such as any of those described herein can be stored in the kits in containers separate from the devices. In addition, the kits can include any device or other element which can facilitate the operation of any aspect of the kits. For example, a kit can comprise the sample preparation body and instructions for use. In some embodiments, a kit can comprise the amplification module and instructions for use. In some embodiments, any combination of the preceding.

In certain embodiments, the kits which are disclosed herein include instructions, such as instructions for using devices or for urinating into a cap and, optionally subsequent use of the device. The instructions for using devices are, in some aspects, recorded on a suitable recording medium. For example, the instructions can be printed on a substrate, such as paper or plastic, etc. As such, the instructions can be present in the kits as a package insert, in the labeling of the container of the kit or components thereof (i.e., associated with the packaging or subpackaging etc.). In other embodiments, the instructions are present as an electronic storage data file present on a suitable computer readable storage medium, e.g., Portable Flash drive, CD-ROM, diskette, on the cloud, etc. The instructions can be storable and/or reproducible within one or more programs, such as computer applications. The instructions can take any form, including complete instructions for how to use the devices or as a website address with which instructions posted on the world wide web can be accessed.

In some embodiments, the amplification module comprises a reusable component, e.g., substrate. In some embodiments, prior to assembly of the devices or systems provided herein, a user obtains the sample preparation device in separate packaging from the amplification module and in separate packaging from the cap. This maintains the sterility of the separate components. In some embodiments, prior to assembly of the devices or systems provided herein, a user obtains the reusable component of the amplification module separate packaging than other system components. This allows the reusable components to be sold separately from the other single-use components.

In some embodiments, a user can obtain the sample preparation device in the same packaging as the amplification module and cap.

### VI. Additional Considerations

All references, issued patents and patent applications cited within the body of the specification are hereby incorporated by reference in their entirety, for all purposes.

The foregoing description of the embodiments of the disclosure has been presented for the purpose of illustration—it is not intended to be exhaustive or to limit the disclosure to the precise forms disclosed. Persons skilled in the relevant art can appreciate that many modifications and variations are possible in light of the above disclosure.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments of the disclosure is intended to be illustrative, but not limiting, of the scope of the disclosure.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Lysis Buffer and Enzyme Experiments

To determine a possible lysis buffer composition comprising sodium hydroxide, various concentrations, ranging between 10 mM to 250 mM NaOH, were tested for their efficiency to lyse and allow for LAMP reaction once buffered.

The results showed better lysis efficiency and amplification reaction for NaOH lysis buffer ranging from 50 mM to 250 mM. The optimal buffer range may shift when other parameters are changed, e.g., enzymes in the lysis buffer or analyte target.

The following enzymes were tested for their performances in amplifying target DNA (Trichomonas vaginalis as it was the best performing assay at the time):
PCR Biosystems IsoFast Bst DNA polymerase
PCR Biosystems IsoFast Warmstart Bst DNA polymerase
Empirical bioscience Bst DNA polymerase
LGC Bst DNA polymerase
New England Biolabs 2.0 Warmstart Bst DNA polymerase
New England Biolabs 3.0 Bst DNA polymerase
New England Biolabs Full Length DNA polymerase

Briefly, enzyme performance was assessed using fluorometric detection of EvaGreen® dye incorporation into the amplified DNA along 30 reaction cycles using a Bioquant-96 RT-PCR detection system. Cycle threshold (Ct) values obtained for each enzyme were used to rank the enzymes, with lower Ct value corresponding to better enzyme performance. When testing for an exemplary pathogens, e.g., Trichomonas vaginalis, Chlamydia trachomatis, and Neisseria gonorrhoeae. NEB 3.0 Bst DNA polymerase and Empirical bioscience Bst DNA polymerase showed the better enzyme performances than others.

### Example 2: Resuspension Buffer Ratios

The enzymes were first assessed for their capacity to amplify target DNA in 250 mM NaOH lysis buffer with their respective recommended supplier buffers. As some buffers showed higher capacity to reduce the pH of final reaction mix, a general buffer with higher Tris-HCl concentration (50 mM final) was tested.

The current assay was performed in following final concentrations:

| | | |
|---|---|---|
| Tris-HCl | 50 | mM |
| (NH4)2SO4 | 30 | mM |
| Tween 20 | 0.1 | % |
| MgSO4 | 6 | mM |

Therefore, the initial Resuspension buffer concentrations before dilution in Lysis buffer were:
For a 1.4 Lysis Resuspension volumetric ratio:

| | | |
|---|---|---|
| Tris-HCI | 62.5 | mM |
| (NH4)2SO4 | 37.5 | mM |
| Tween 20 | 0.125 | % |
| MgSO4 | 7.5 | mM |

For a 1:1 Lysis: Resuspension volumetric ratio:

| | | |
|---|---|---|
| Tris-HCl | 100 | mM |
| (NH4)2SO4 | 60 | mM |
| Tween 20 | 0.2 | % |
| MgSO4 | 12 | mM |

The pH of different combination of lysis buffer and resuspension buffer was measured and the results are shown below.

| | NaOH concentration | Measured pH |
|---|---|---|
| lysis buffer alone | 250 mM | 12.7 |
| | 100 mM | 12.6 |
| lysis + Resuspension buffer | 250 mM | 9.3 |
| (1:4 ratio) | 100 mM | 8.7 |
| lysis + Resuspension buffer | 250 mM | 12.8 |
| (1:1 ratio) | 100 mM | 9.3 |

### Example 3: Filter Pressure Testing with Urine Samples

### MCE Membrane Filters

The purpose of this study was to determine the pressure difference between donors when using MCE membranes. Pressure reduction had been shown when using 0.65 μm MCE membranes in comparison with the previously used 0.45 μm CA membranes. Those experiments were conducted with only one donor. This study was conducted using the urine of multiple donors to see the expected range of variability between donors. The urine was spiked and pressure was measured for each donor using the 0.45, 0.65 and 0.8 μm MCE membrane. Samples were prepared according to “SOP-Prototype-Sample-Preparation-V1.3” using the pressure sensor device.

Below are the results from the experiment:

| | Donor [—] | | | | | | | | | | | | | |
|---|---|---|---|---|---|---|---|---|---|---|---|---|---|---|
| | 1 | | | 2 | | | 3 | | 4 | | | 5 | | |
| | Membrane type [—] | | | | | | | | | | | | | |
| | MCE | | | MCE | | | MCE | | MCE | | | MCE | | |
| Pore sizes | 0.45 | 0.65 | 0.8 | 0.45 | 0.65 | 0.8 | 0.65 | 0.8 | 0.45 | 0.65 | 0.8 | 0.45 | 0.65 | 0.8 |
| [um] | | | | | | | | | | | | | | |
| Volume | 3.4 | 2.4 | 1.8 | 3.2 | 2.2 | 2.2 | 3.2 | 3.0 | 4.0 | 3.4 | 3.0 | 3.4 | 3.0 | 2.4 |
| measured | | | | | | | | | | | | | | |
| [ml] | | | | | | | | | | | | | | |
| Height | 29 | 23 | 15 | 26 | 18 | 18 | 26 | 24 | 33 | 29 | 24 | 29 | 24 | 23 |
| measured | | | | | | | | | | | | | | |
| [mm] | | | | | | | | | | | | | | |
| Pressure | 3.6 | 2.3 | 1.6 | 2.8 | 1.8 | 1.8 | 2.8 | 2.5 | 5.7 | 3.6 | 2.5 | 3.6 | 2.5 | 2.3 |
| measured | | | | | | | | | | | | | | |
| [bar] | | | | | | | | | | | | | | |

It was not possible to conduct the 0.45 μm membrane assay with donor 3 since the first membrane broke and no more urine was available.

Data from the previous experiments (10.08, 15.08) using the MCE membranes were added (donor 6 and 7) to further show the distribution of variability.

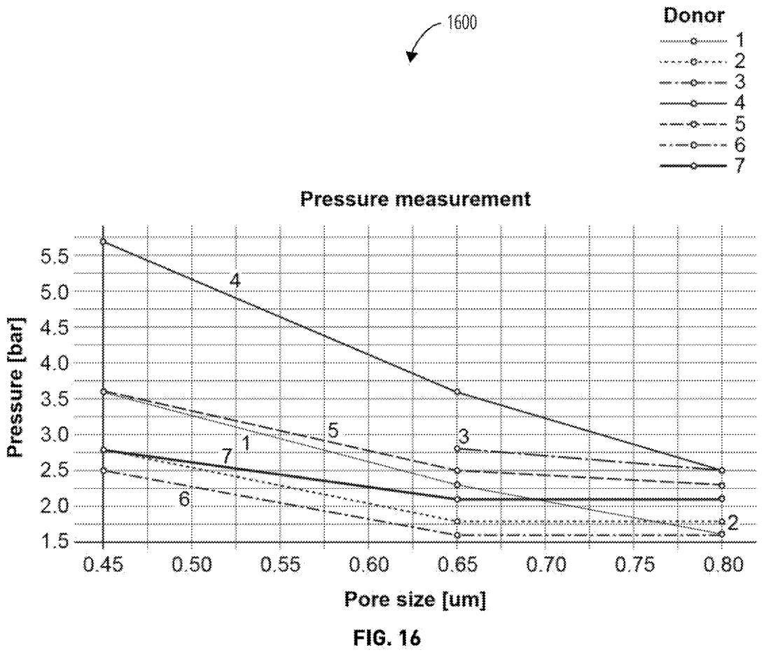

The results are shown in FIG. 16. Overall, the results indicated that an increase of the pore size results in a significant decrease of pressure between 0.45 and 0.65 while the reduction to 0.8 does not have a significant influence on an average urine sample. The impact was greater in concentrated samples.

The variability obtained with the 0.65 membrane is of around 1.8 bars with the different donors. Donors 1, 5 and 7 were probably close to what an average urine in term of what can be expected as concentration while donor 5 was above and donor 6 was below. Pressures encountered here (even the higher ones) caused no difficulty for the experimenter to screw the prototype completely.

The results indicate that the pressure is significantly lowered when using the 0.65 membranes in comparison to the previously used 0.45 μm CA membrane which returned pressures as high as 6.7 bars in the most extreme cases. Furthermore, with the 0.65 MCE membrane, it is highly unlikely that it will go above 5 bars, which is still lower than the intended usage limit of 10 bars.

### Glass Filters

A study was conducted to test the pressure obtained using GF/F filters in comparison with 0.65 μM MCE filters. To do so, urine was filtered through GF/F and 0.65 μm MCE filters and pressure was measured using a pressure measurement device.

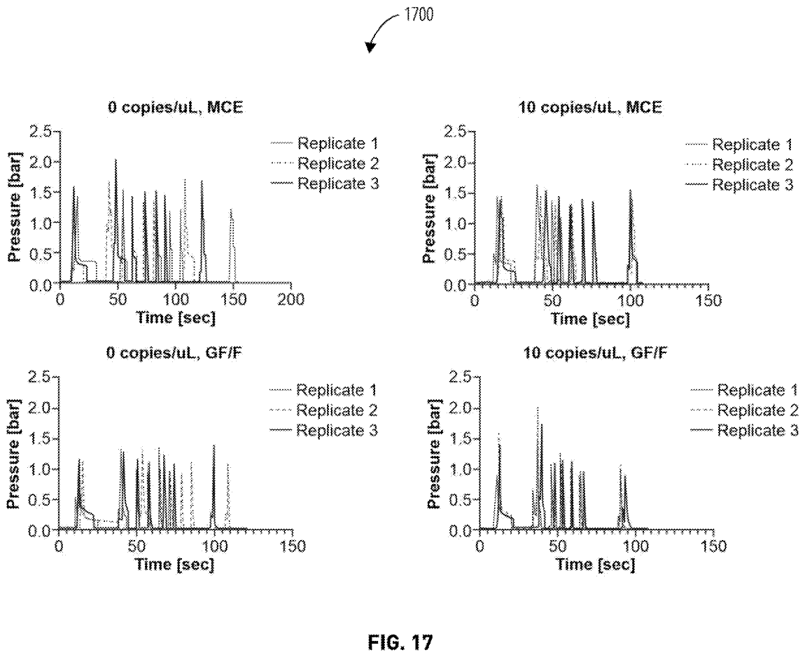

The pressure data results are shown in FIG. 17. Regarding pressure, maximal pressure reached was similar in both filter types (around 2 bar). The overall pressure is lower in GF/F than 0.65 μm MCE.

### Example 4: Urine Collection and Testing System Usability

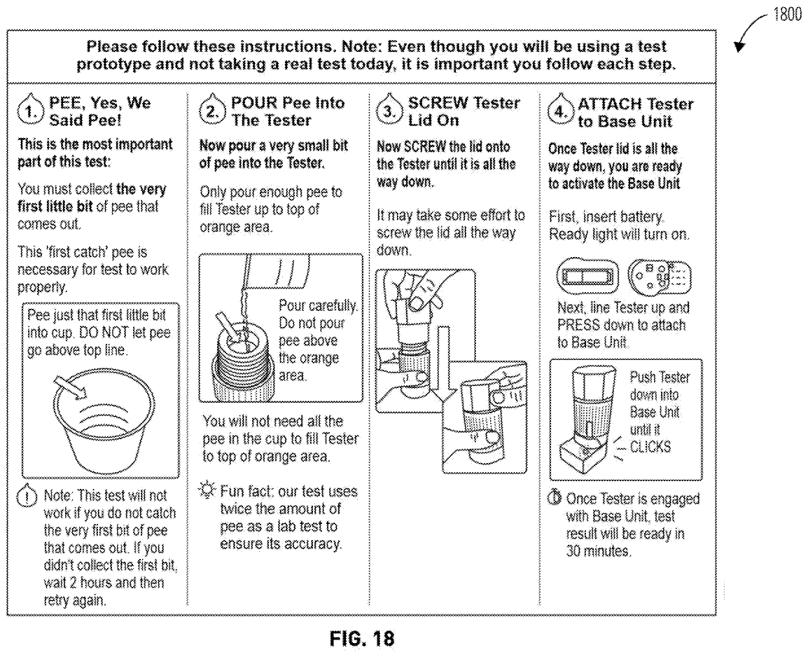

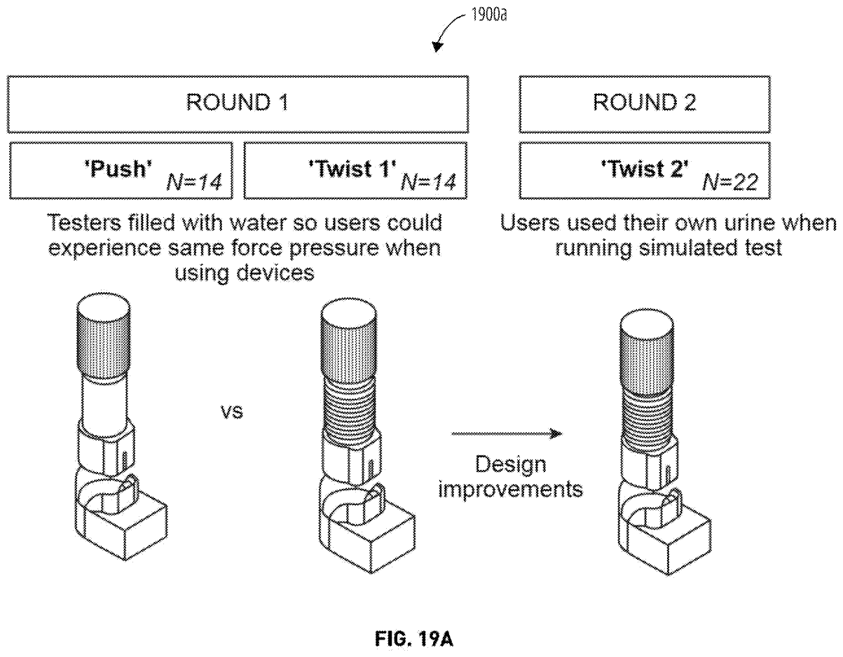

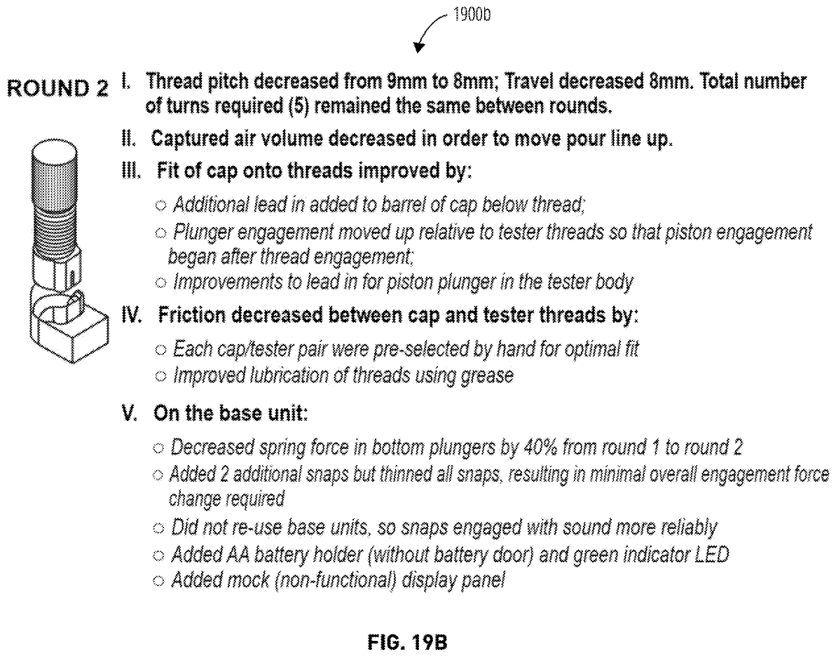

Fifty subjects (round 1: n=28, round 2: n=22) were instructed to collect a urine sample, pour a volume of the urine sample it into the sample preparation device, screw the cap and then engage the sample preparation body to the amplification module, as outlined in FIG. 18. The differences in the rounds are shown in FIGS. 19A AND 19B. After completing the analyte test using the sample preparation system as disclosed herein, the users were question on urine collection, transfer of urine to the sample preparation device, and use of the device to results.

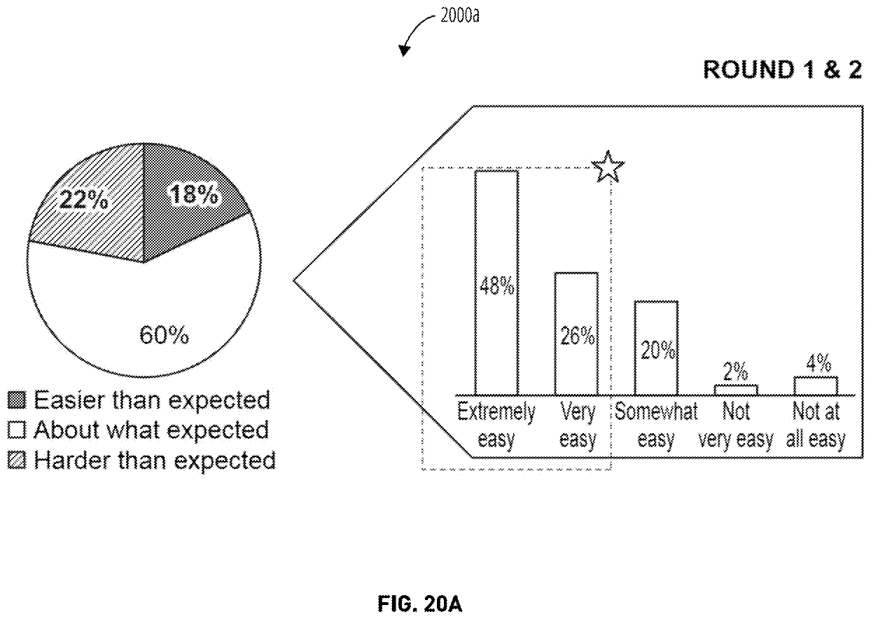

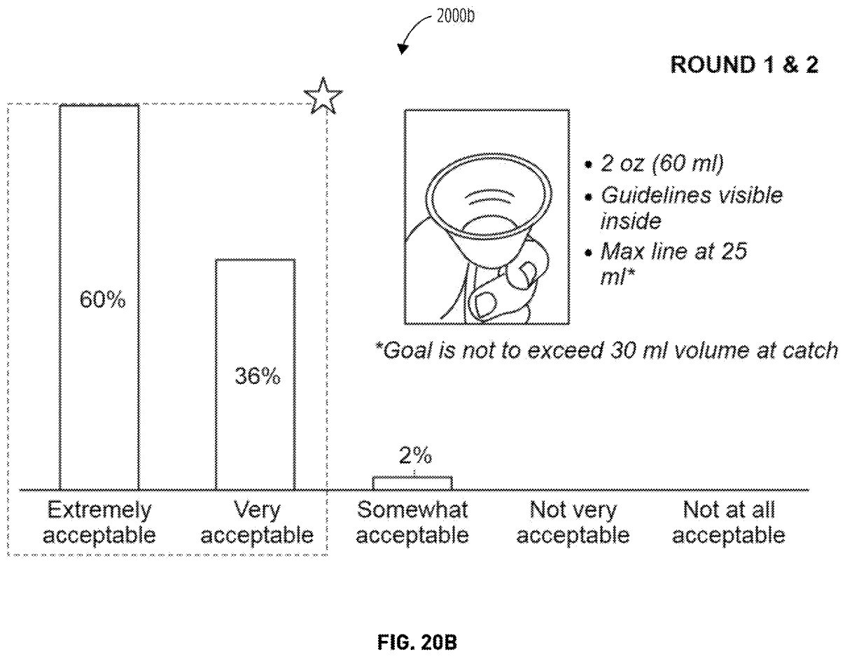

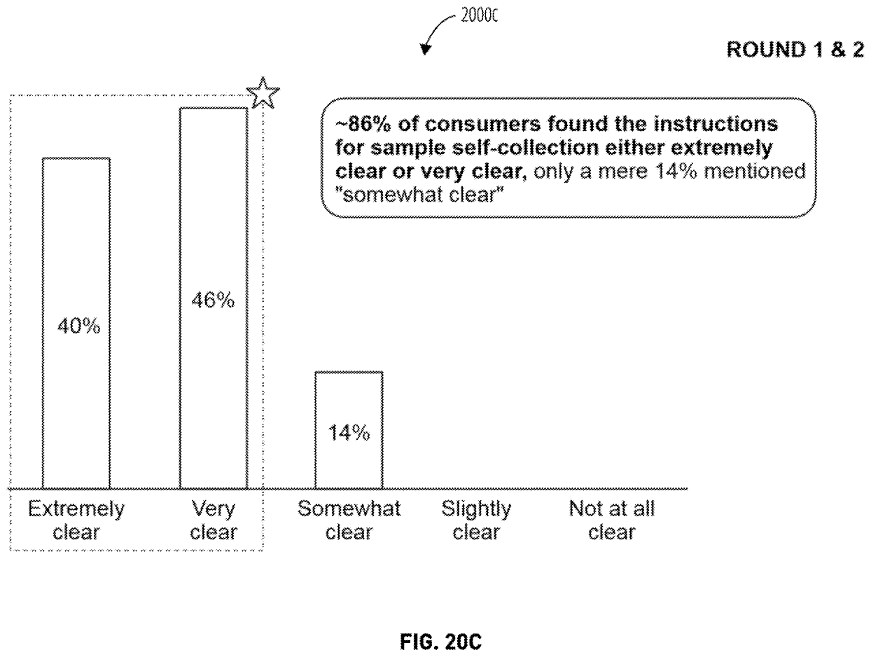

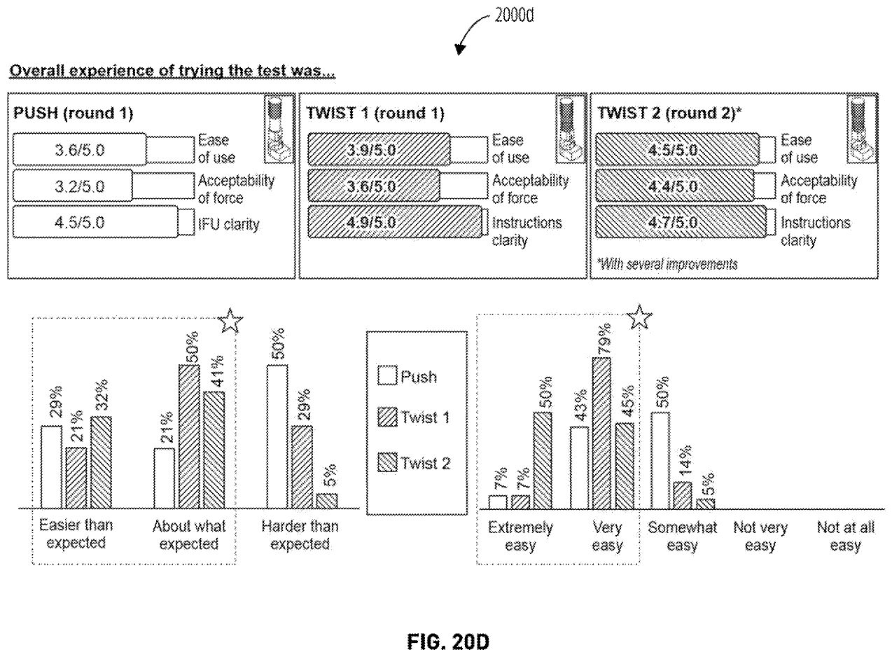

The results of this study are shown in FIGS. 20A-20D. Most of the subjects considered the urine collection process very easy, if not extremely easy. The majority found it acceptable to be asked to void a small amount in the cup (as instructed) and found the instructions at least very clear. The users were all able to perform the urine transfer into the sample preparation device by pouring, which, in some embodiments of the present disclosure, would allow simple pouring of the sample rather than more complex transfer techniques (e.g., pipetting). FIG. 20D shows the user's feedback on ease of use for different sample preparation device systems (e.g., push cap vs. twist cap) and the ratings for the users' overall experience using a presently disclosed sample preparation system.

Most users (~96%) displayed a high level of comfort towards home testing and indicated (~86%) their likelihood of using such an at-home test. Most also indicated that if they had access to the presently disclosed device for testing STDs, they would test every 1-3 months (the CDC recommendation).

What is claimed is:

1. A sample preparation device for processing biological samples for testing for a disease or condition, the device comprising:
(a) a sample preparation body comprising:
(i) a sample chamber for holding sample fluid,
(ii) a filter housing fluidically connected to the sample chamber,
optionally wherein the filter housing comprises a filter, and
(iii) a second chamber comprising one or more plungers disposed therein to form a plurality of chambers, wherein the plurality of chambers each comprises at least one of a reagent, aqueous fluid, or air, wherein the second chamber comprises an opening fluidically connecting the filter housing and the second chamber, wherein the opening is configured to be opened and closed upon movement of the one or more plungers through the second chamber so as to selectively dispense contents of the plurality of chambers into the filter housing and through the filter housing; and
(b) a cap comprising a filter plunger, wherein the cap is configured for mounting on an open end of the sample preparation body, and wherein the filter plunger is configured to compress the sample fluid through the filter housing, optionally through though the filter in the filter housing.

2. The device of claim 1, wherein the second chamber comprises two or more plungers.

3. The device of claim 1 or 2, wherein the sample preparation device has a reception configuration and a preparation configuration, the device being movable from the reception configuration, in which a sample fluid can be introduced into the sample preparation body, to the preparation configuration, in which the sample preparation body is closed by assembly of the cap to the sample preparation body and the user compresses the cap against the sample preparation body to actuate the one or more plungers.

4. The device of any one of claims 1-3, wherein the sample chamber is disposed within the second chamber, optionally, wherein the sample chamber and the second chamber are concentric.

5. The device of any one of the preceding claims, further comprising a waste chamber.

6. The device of any one of the preceding claims, further comprising a prepared sample chamber comprising (i) a breakable barrier and (ii) an outlet port.

7. The device of claim 6, wherein the prepared sample chamber is disposed within the waste chamber, and the breakable barrier seals the prepared sample chamber from the waste chamber.

8. The device of any one of the preceding claims, wherein the sample chamber comprises an outlet fluidically connecting the sample chamber and the filter housing, optionally wherein the outlet comprises a breakable barrier.

9. The device of any one of the preceding claims, wherein the filter housing comprises an outlet (optionally a tapered outlet).

10. The device of claim 9, wherein the outlet is a hollow piercing member.

11. The device of claim 9 or 10, wherein the outlet is aligned to operatively connect with the breakable barrier in a configuration, thereby breaking the prepared sample chamber's breakable barrier and fluidically connecting the filter housing to the prepared sample chamber.

12. The device of claim 9 or 10, wherein the outlet is configured to operatively connect with the breakable barrier, optionally upon further closure of the cap, thereby breaking the prepared sample chamber's breakable barrier and fluidically connecting the filter housing to the prepared sample chamber.

13. The device of any one of claims 6-12, further comprising a valve configured such that when a user actuates the valve, the valve operatively couples the filter housing to the prepared sample chamber, thereby fluidically connecting the filter housing to the prepared sample chamber.

14. The device of any one of claims 6-13 wherein the prepared sample chamber is configured to be compressed against the filter housing, thereby breaking the prepared sample chamber's breakable barrier and fluidically connecting the filter housing to the prepared sample chamber.

15. The device of any one of the preceding claims, wherein at least one of the plurality of chambers comprises a reagent.

16. The device of claim 15, wherein the reagent comprises one or more of a washing buffer, a lysis buffer, a neutralization buffer, an elution buffer, or a rehydration buffer.

17. The device of claim 15, wherein the reagent comprises a lysis buffer.

18. The device of any one of the preceding claims, wherein at least one of the plurality of chambers comprises aqueous fluid.

19. The device of any one of the preceding claims, wherein at least one of the plurality of chambers comprises air.

20. A sample preparation system for processing biological samples for testing for a disease or condition, the system comprising:
(a) a sample preparation body comprising an inner body, an outer body, a filter housing, a waste chamber, and a reagent chamber,
wherein the inner body comprises a sample chamber for holding sample fluid,
wherein the filter housing is fluidically connected to the sample chamber,
wherein the waste chamber comprises a prepared sample chamber,
wherein the prepared sample chamber comprises (i) a breakable barrier sealing it from the waste chamber and (ii) an outlet port,
wherein the filter housing comprises (i) an first end, (ii) a filter, and (iii) a second end, wherein the second end comprises an outlet (optionally a tapered outlet) configured to allow filtrate flow therethrough,
wherein the outer body comprises one or more plungers disposed therein to form at least one aqueous fluid chamber and/or at least one air chamber;
(b) an amplification module operatively coupled to the sample preparation body and adjacent to the prepared sample chamber,
wherein the amplification module comprises an interface and a reagent plunger configured to move within the reagent chamber,
wherein the outlet port of the prepared sample chamber is configured to receive the interface, thereby fluidically connecting the prepared sample chamber and the amplification module, and
wherein the reagent plunger is configured to move within the reagent chamber; and
(c) a cap configured for mounting on the open end of the sample preparation body, wherein the cap comprises a filter plunger aligned to enter the open end of the sample chamber when the sample preparation body is closed by the assembly of the cap to the sample preparation body;
wherein the sample preparation system has a reception configuration, a filtering configuration, and a reagent configuration, the system being movable from the reception configuration in which the sample fluid can be introduced into the sample chamber, to the filtering configuration in which sample preparation body is closed by the assembly of the cap to the sample preparation body, to the reagent configuration,
wherein in the filtering configuration, the filter plunger pressurizes the sample chamber, thereby driving the sample fluid from the sample chamber through the filter housing and into the waste chamber, and wherein further compressing the cap against the sample preparation body moves the one or more plungers to open and close the outer body (optionally through an opening on the outer body wall) so as to selectively dispense the content of the at least one aqueous fluid chamber and/or the at least one air chamber through the filter housing and into the waste chamber,
wherein in the reagent configuration, further compressing the cap against the sample preparation body operatively couples the second end and prepared sample chamber, thereby breaking the breakable barrier and fluidically connecting the reagent chamber, the filter housing, and the prepared sample chamber, and further compressing the cap moves the reagent plunger into the reagent chamber, thereby driving the contents of the reagent chamber through the filter housing to the prepared sample chamber.

21. The sample preparation system of claim 20, wherein the reagent chamber further comprises a second plunger, thereby forming a plurality of chambers each having one or more reagents, aqueous fluid, or air, and
wherein in the reagent configuration, the further compressing the cap selectively dispenses the contents of the plurality of chambers through the filter housing and prepared sample chamber.

22. The sample preparation system of claim 21, wherein the plurality of chambers comprise at least one chamber having a reagent and at least one chamber having air.

23. The sample preparation system of claim 22, wherein as the reagent plunger moves into the reagent chamber, the contents of a chamber having air are driven through the filter housing after the contents of a chamber having a reagent are driven through the filter housing.

24. The sample preparation system of any one of claims 20-23, wherein the contents of the air chamber are driven through the filter housing and the prepared sample chamber after the contents of the aqueous fluid chamber are driven through the filter housing.

25. The sample preparation system or device of any one of the preceding claims, wherein the filter of the filter housing has a pore size suitable for allowing fluid flow therethrough and for obstructing the flow of cells.

26. The sample preparation system or device of claim 25, wherein the cells are bacteria, fungi, or protists.

27. The sample preparation system or device of claim 25, wherein the cells are cancerous cells and/or precancerous cells.

28. The sample preparation system of any one of claims 20-27, wherein the reagent chamber comprises a vent.

29. The sample preparation system of claim 28, wherein the vent has passively tunable porosity.

30. The sample preparation system or device of any one of the preceding claims, wherein the cap is a screw cap.

31. The sample preparation system or device of any one of the preceding claims, wherein the cap comprises a first attachment element and the sample preparation body has a second attachment element.

32. The sample preparation system or device of claim 30 or 31, wherein the first attachment element comprises threading and the second attachment element comprises reciprocating threading configured to slidably receive the threading.

33. The sample preparation system or device of claim 32, wherein the reciprocating threading is configured to slidably receive ½ to 10 complete rotations of the threading of the cap.

34. The sample preparation system or device of claim 32 or 33, wherein the reciprocating threading is configured to slidably receive at least 3 complete rotations of the threading of the cap to complete the filtering configuration or the preparation configuration.

35. The sample preparation system or device of any one of claims 32-34, wherein the reciprocating threading is configured to slidably receive at least 5 complete rotations of the threading of the cap to complete the filtering configuration or the preparation configuration.

36. The sample preparation system or device of any one of claims 32-34, wherein the reciprocating threading is configured to slidably receive 3 to 5 complete rotations of the threading of the cap to complete the filtering configuration or the preparation configuration.

37. A sample preparation device for processing biological samples for testing for a disease or condition, the device comprising:
a sample preparation body comprising:
(vi) a sample chamber for holding sample fluid,
(vii) a filter housing fluidically connected to the sample chamber;
(ii) a second chamber comprising one plunger disposed therein to form a plurality of chambers, wherein the plurality of chambers each comprise at least one of a reagent, aqueous fluid, or air, and wherein the second chamber comprises an opening fluidically connecting the sample chamber and the second chamber, wherein the opening is configured to be opened and closed upon movement of the plunger through the second chamber so as to selectively dispense contents of the plurality of chambers into the sample chamber and through the filter;
(iii) a cap comprising a filter plunger, wherein the cap is configured for mounting on an open end of the sample preparation body, wherein the filter plunger is configured to compress the sample fluid against the filter; and
(iv) a reagent chamber comprising one plunger disposed therein to form a plurality of chambers, wherein the plurality of chambers each comprise (a) one chamber of a reagent, aqueous fluid, or air and (b) one waste chamber.

38. The sample preparation device of claim 37, wherein the second chamber comprises two or more chambers disposed therein to form a plurality of chambers.

39. The sample preparation device of claim 37, wherein the reagent chamber comprises two or more plungers disposed therein to form a plurality of chambers.

40. The sample preparation device of claim 37, wherein the plurality of chambers in the reagent chamber comprise (a) one chamber of reagent and (b) one waste chamber.

41. The sample preparation system or device of any one of the preceding claims, wherein the outlet comprises a hydrophilic material.

42. The sample preparation system or device of any one of the preceding claims, wherein the breakable barrier comprises a hydrophilic material.

43. The sample preparation system or device of any one of the preceding claims, wherein the outlet comprises a material having a hardness that is at least 2 times a hardness of a material comprising the breakable barrier, such that the outlet provides for a clear puncture of the breakable seal with repeatable geometry and without deformation of the puncturing element.

44. The sample preparation system or device of any one of the preceding claims, wherein the cap comprises knurlings and/or one or more finger-grip indentations.

45. The sample preparation system or device of any one of the preceding claims, wherein the outer surface of the sample preparation body and/or the amplification module comprises one or more alignment features.

46. The sample preparation system or device of any one of the preceding claims, wherein the cap, the sample preparation tube and/or the amplification module comprise irreversible coupling features (such as locking detents, cantilever snap features, lock-out release arm, ratchet features, sawtooth ratchets, ratchet and pawl features).

47. The sample preparation system or device of any one of the preceding claims, wherein the sample fluid comprises a urine sample, a saliva sample, and/or a mouthwash sample.

48. The sample preparation system or device of any one of the preceding claims, wherein the sample fluid comprises a urine sample.

49. The sample preparation system or device of any one of claims 1-48, wherein the sample fluid comprises a fecal sample or mucosal sample.

50. The sample preparation system or device of any one of the preceding claims, wherein the sample fluid comprises an analyte.

51. The sample preparation system or device of any one of the preceding claims, wherein the disease or condition is a sexually transmitted disease, an oncological condition, a fungal infection, and/or a bacterial infection.

52. The sample preparation system or device of any one of the preceding claims, wherein the filter comprises an affinity capture reagent membrane.

53. The sample preparation system or device of claim 52, wherein the filter comprises an anion exchange membrane.

54. The sample preparation system or device of claim 52, wherein the filter comprises a cation exchange membrane.

55. The sample preparation system or device of any one of the preceding claims, wherein the filter comprises a target-specific binding moiety.

56. The sample preparation system or device of any one of the preceding claims, wherein the filter comprises protein A.

57. The sample preparation system or device of any one of the preceding claims, wherein the filter comprises protein G.

58. The sample preparation system or device of any one of the preceding claims, wherein the filter comprises protein L.

59. The sample preparation system or device of any one of the preceding claims, wherein the filter is infused with a salt.

60. The sample preparation system or device of any one of the preceding claims, wherein the filter has a pore size no greater than 0.7 μm, 0.6 μm, 0.5 μm, 0.45 μm, 0.4 μm, 0.2 μm, or 0.22 μm.

61. The sample preparation system or device of any one of the preceding claims, wherein the reagent comprises a lysis buffer.

62. The sample preparation system or device of claim 61, wherein the lysis buffer comprises at least one of sodium hydroxide, sodium dihydrogen phosphate, disodium hydrogen phosphate, Tris, HEPES, and detergent.

63. The sample preparation system or device of any one of the preceding claims, wherein the reagent comprises a neutralization reagent.

64. The sample preparation system or device of claim 63, wherein the neutralization reagent comprises an alkaline reagent and/or wherein the neutralization buffer comprises one or more of Tris, Tris-HCl, and ethylenediaminetetraacetic acid (EDTA).

65. The sample preparation system or device of any one of the preceding claims, wherein the amplification module comprises LAMP primers.

66. A method of determining whether a user has a disease or condition, comprising:
(i) providing a sample preparation system or device of any one of the preceding claims;
(ii) providing a biological sample;
(iii) filtering at least a portion of the biological sample through the filter, thereby retaining an analyte, comprising nucleic acid molecules, on the filter;
(iv) applying at least one reagent to the filter, thereby producing a lysate, wherein the one reagent comprises a lysis buffer;
(v) amplifying the nucleic acid molecules of the analyte, and based on said amplifying;
(vi) determining the presence or absence of the analyte.

67. A method of determining whether a user has a disease or condition, the method comprising:
(i) providing a sample preparation device having a filter housing that comprises a filter and is fluidically connected to the sample chamber;
(ii) providing a urine sample that was collected by having the user micturate into a sample collector by initiating and then stopping their flow of urine into the sample collector, wherein the sample collector has a volume of no more than about 70 mL, optionally about 20 mL, 30 mL, 40 mL, 50 mL or 60 mL;
(iii) pouring a predetermined volume from the urine sample into the sample preparation device and filtering the predetermined volume, thereby retaining an analyte on the filter, wherein the predetermined volume is no more than about 5 mL;
(iv) applying a lysing agent to the filter, thereby producing a lysate comprising nucleic acid molecules;
(v) amplifying nucleic acid molecules, and based on said amplifying;
(vi) determining the presence or absence of a pathogen, cancerous cell, and/or precancerous cell.

68. A method of determining whether a user has a disease or condition, the method comprising:
(i) providing sample preparation device or system of any one of claims 1-61;
(ii) providing a urine sample that was collected by having the user micturate into a sample collector by initiating and then stopping their flow of urine into the sample collector, wherein the sample collector has a volume of no more than about 70 mL, optionally about 20 mL, 30 mL, 40 mL, 50 mL or 60 mL;
(iii) pouring a predetermined volume from the urine sample into the sample chamber and filtering the predetermined volume, thereby retaining an analyte on the filter, wherein the predetermined volume is no more than about 5 mL;
(iv) applying a lysing agent to the filter, thereby producing a lysate comprising nucleic acid molecules;
(v) amplifying nucleic acid molecules, and based on said amplifying;
(vi) determining the presence or absence of a pathogen, cancerous cell, and/or precancerous cell.

69. The method of claim 67 or 68, wherein the sample collector has a volume of no more than about 15 mL.

70. The method of claim 67 or 68, wherein the sample collector has a volume of no more than about 10 mL.

71. The method of any one of claims 67-68, wherein the predetermined volume is about 3 mL.

72. The method of any one of claims 67-68, wherein the predetermined volume is about 4 mL.

73. The method of any one of claims 67-68, wherein the predetermined volume is about 5 mL.

74. The method of any one of claims 67-73, wherein the inner surface of the sample collector comprises at least two indicators.

75. The method of any one of claims 67-74, wherein the pathogen is a bacterium, fungus, protist, or virus that is capable of causing sexually transmitted infections.

76. The method of any one of claims 67-74, wherein the pathogen is a bacterium, fungus, protist, or virus that is capable of causing a urinary tract infection.

77. The method of any one of claims 67-74, wherein determining presence or absence of a pathogen, cancerous cell, and/or precancerous cell comprises using a lateral flow assay (LFA).
